(19) United States
(12) Patent Application Publication
(10) Pub. No.: US 2026/0240854 A1
(43) Pub. Date: Aug. 20, 2026

(54) COMBINATIONS OF KRAS INHIBITOR AND ATR INHIBITOR FOR THE TREATMENT OF CANCER

(71) Applicant: ASTRAZENECA AB, Södertälje (SE)

(72) Inventors: Alexander KOERS, Cambridge (GB); Alan LAU, Cambridge (GB); Sarah ROSS, Cambridge (GB)

(21) Appl. No.: 19/470,877

(22) PCT Filed: Apr. 12, 2024
(86) PCT No.: PCT/EP2024/059956

§ 371 Date: Sep. 30, 2025

Related U.S. Application Data
(60) Provisional application No. 63/496,040, filed on Apr. 14, 2023.

(51) Int. Cl. A61K 31/519; A61K 31/5377; A61P 35/00

(52) U.S. Cl. CPC A61K 31/519; A61K 31/5377; A61P 35/00

(57) ABSTRACT

The present provides a method of treating cancer in a subject, the method comprising administering to the subject: (a) a KRAS inhibitor, and (b) an Ataxia Telangiectasia and Rad-3-related (ATR) inhibitor. Also disclosed are compositions and kits comprising (a) a KRAS inhibitor, and (b) an ATR inhibitor.

The present specification claims benefit to U.S. Provisional Application No. 63/496,040, filed 14 Apr. 2023, the content of which is hereby incorporated by reference in its entirety for all purposes.

### FIELD

The present disclosure provides a method of treating cancer in a subject, the method comprising administering to the subject: (a) a KRAS inhibitor, and (b) an Ataxia Telangiectasia and Rad-3-related (ATR) inhibitor. Also disclosed are compositions and kits comprising (a) a KRAS inhibitor, and (b) an ATR inhibitor.

### BACKGROUND

KRAS is a small GTPase protein which cycles between GDP-bound inactive & GTP-bound active states to regulate cellular signaling cascades and promote cell proliferation and survival. KRAS is frequently mutated in cancer with gain-of-function missense mutations clustering in hotspots, e.g., at codon 12, 13 and 61. Oncogenic mutations at these residues can disrupt intrinsic and GAP-mediated GTP hydrolysis increasing levels of GTP-bound active KRAS, thereby leading to inappropriate activation of cellular signaling cascades which can drive the progression of cancers.

Historically KRAS was considered an intractable target, due to lack of druggable pockets and high affinity binding to nucleotide. However, some small molecules with improved potency and pharmacological properties have been developed, which covalently bind the mutant cysteine residue in KRAS trapping it in a GDP-bound inactive state and driving anti-tumor activity in KRAS preclinical models, e.g., sotorasib and adagrasib. However, not all patients with KRAS mutant tumors respond robustly to treatment.

Ataxia telangiectasia and Rad3-related (ATR) is a serine/threonine protein kinase which is the apical kinase involved in coordinating cell cycle checkpoints and the DNA damage response induced by DNA replication stress. Inhibition of ATR has been explored as a potential treatment for cancer initially focused on targeting tumor intrinsic mechanisms with enhanced sensitivity to ATR inhibition due to defective DNA-double strand break (DSB) repair mechanisms. Several ATR inhibitors are known, including AZD6738 (ceralasertib, (Foote KM., et al, J Med Chem. 2018; 61(22):9889-907)), M6620/VX970 (berzosertib, (Gorecki L, et al., Pharmacol Ther. 2020; 210:107518)), and BAY-1895344 (elimusertib, (Lucking U, et al., J Med Chem. 2020; 63(13):7293-325)).

### SUMMARY

In some embodiments, the disclosure provides a method of treating cancer in a subject, the method comprising administering to the subject: (a) a KRAS inhibitor, and (b) an Ataxia Telangiectasia and Rad-3-related (ATR) inhibitor.

In some embodiments, the disclosure provides a KRAS inhibitor for use in the treatment of cancer in a subject, wherein said treatment comprises the separate, sequential or simultaneous administration of:
a. said KRAS inhibitor, and
b. an ATR inhibitor,
   to said subject.

In some embodiments, the disclosure provides an ATR inhibitor for use in the treatment of cancer in a subject, wherein said treatment comprises the separate, sequential or simultaneous administration of:
a. a KRAS inhibitor, and
b. said ATR inhibitor, to said subject.

In some embodiments, the disclosure provides use of a KRAS inhibitor or an ATR inhibitor in the manufacture of a medicament for administration of the KRAS inhibitor and the ATR inhibitor in combination, for the treatment of cancer in a subject.

In some embodiments, the KRAS inhibitor is an antibody. In some embodiments, the antibody is ELI-002, KRAS-EphA-2-CAR-DC, Anti-KRAS G12V mTCR PBL, anti-KRAS G12D mTCR PBL, siG12D-LODER, or KRAS G12D siRNA. In some embodiments, the KRAS inhibitor is a small molecule KRAS inhibitor. In some embodiments, the small molecule KRAS inhibitor is sotorasib, adagrasib, Ly3537982, GDC-6036, D-1553, JDQ443, BI1823911, GFH925, D35-001, JAB-21822, HBI-2438, YL-15293, GEC255, IBI351, RMC-6291, MK-1084, MRTX1133, HRS-4642, RMC-9805, JAB-2200, Vrtx153, ERAS-4, ASP3082, RMC-0708, RMC-8839, RMC-6291, JAB-23400, TEB-17231, or QTX3046.

In some embodiments, the small molecule KRAS inhibitor is a compound of the Formula (I):

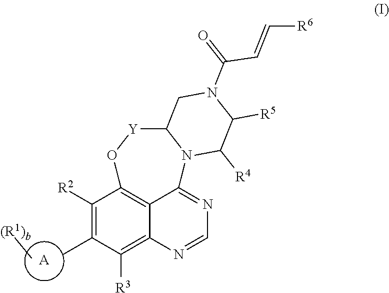

as defined herein. In some embodiments, the small molecule KRAS inhibitor has the structure:

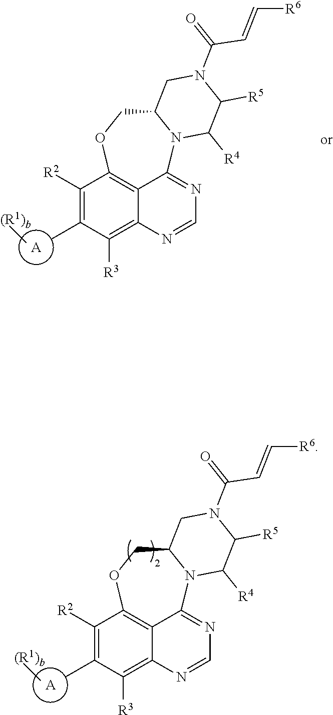

as defined herein. In some embodiments, the ring A is selected from the group consisting of:

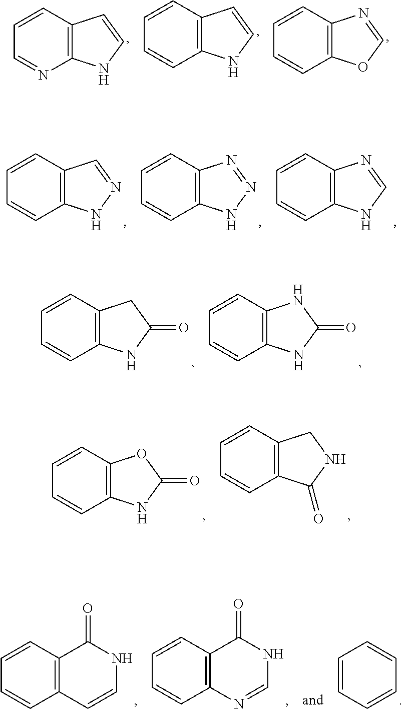

In some embodiments, the ring A as listed above is attached to the remainder of compound for Formula (I) at any point on the above listed rings.

In some embodiments, R⁴ is H. In some embodiments, R⁶ is H. In some embodiments, Y is CH₂. In some embodiments, Y is CH₂CH₂. In some embodiments, R² is Cl. In some embodiments, R³ is F. In some embodiments, R⁴ is H and R⁵ is Me. In some embodiments, the small molecule KRAS inhibitor is selected from:
7-[(8aS)-10-Acryloyl-6-chloro-4-fluoro-8,8a,9,10,11,12-hexahydropyrazino[2′,1′:3,4][1,4]oxazepino [5,6,7-de]quinazolin-5-yl]-6-methyl-2,3-dihydro-1H-isoindol-1-one;
1-[(8aS,11S)-6-Chloro-4-fluoro-5-(2-fluoro-6-hydroxyphenyl)-11-methyl-8a,9,11,12-tetrahydropyrazino[2′,1′:3,4][1,4]oxazepino[5,6,7-de]quinazolin-10(8H)-yl]prop-2-en-1-one;
1-[(8aS,11R)-6-Chloro-4-fluoro-5-(2-fluoro-6-hydroxyphenyl)-11-methyl-8a,9,11,12-tetrahydropyrazino[2′,1′:3,4][1,4]oxazepino[5,6,7-de]quinazolin-10(8H)-yl]prop-2-en-1-one;
5-[(8aS)-10-Acryloyl-6-chloro-4-fluoro-8,8a,9,10,11,12-hexahydropyrazino[2′,1′:3,4][1,4]oxazepino [5,6,7-de]quinazolin-5-yl]-6-methylquinazolin-4(3H)-one;
1-[(8aS)-6-Chloro-4-fluoro-5-(5-methyl-1H-benzimidazol-4-yl)-8a,9,11,12-tetrahydropyrazino [2′,1′:3,4][1,4]oxazepino[5,6,7-de]quinazolin-10(8H)-yl]prop-2-en-1-one;
8-[(8aS)-6-Chloro-4-fluoro-10-(prop-2-enoyl)-8,8a,9,10,11,12-hexahydropyrazino[2′,1′:3,4][1,4]oxazepino[5,6,7-de]quinazolin-5-yl]isoquinolin-1(2H)-one;
1-[(8aS)-6-Chloro-4-fluoro-5-(1H-indazol-3-yl)-8a,9,11,12-tetrahydropyrazino[2′,1′:3,4][1,4]oxazepino[5,6,7-de]quinazolin-10(8H)-yl]prop-2-en-1-one;
1-[(8aS)-6-Chloro-4-fluoro-5-(2-hydroxy-6-methylphenyl)-8a,9,11,12-tetrahydropyrazino[2′,1′:3,4][1,4]oxazepino[5,6,7-de]quinazolin-10(8H)-yl]prop-2-en-1-one;
(2E)-1-[(8aS)-6-Chloro-4-fluoro-5-(2-fluoro-6-hydroxyphenyl)-8a,9,11,12-tetrahydropyrazino [2′,1′:3,4][1,4]oxazepino[5,6,7-de]quinazolin-10(8H)-yl]-4-(dimethylamino)but-2-en-1-one;
8-[(8aS)-6-Chloro-4-fluoro-10-(prop-2-enoyl)-8,8a,9,10,11,12-hexahydropyrazino[2′,1′:3,4][1,4]oxazepino[5,6,7-de]quinazolin-5-yl]-7-methylisoquinolin-1(2H)-one;
1-[(8aS)-6-Chloro-4-fluoro-5-(5-methyl-1H-benzotriazol-4-yl)-8a,9,11,12-tetrahydropyrazino [2′,1′:3,4][1,4]oxazepino[5,6,7-de]quinazolin-10(8H)-yl]prop-2-en-1-one;
1-((8aS)-6-chloro-4-fluoro-5-(5-fluoro-1H-benzo[d]imidazol-4-yl)-8a,9,11,12-tetrahydropyrazino [2′,1′:3,4][1,4]oxazepino[5,6,7-de]quinazolin-10(8H)-yl)prop-2-en-1-one;
1-[(8aS)-6-Chloro-4-fluoro-5-(5-fluoro-1H-indazol-4-yl)-8a,9,11,12-tetrahydropyrazino[2′,1′:3,4][1,4]oxazepino[5,6,7-de]quinazolin-10(8H)-yl]prop-2-en-1-one;
1-((8aS)-6-Chloro-4-fluoro-5-(5-fluoro-1-methyl-1H-benzo[d]imidazol-4-yl)-8a,9,11,12-tetrahydropyrazino[2′,1′:3,4][1,4]oxazepino[5,6,7-de]quinazolin-10(8H)-yl)prop-2-en-1-one;
1-[(8aS)-6-Chloro-4-fluoro-5-(5-fluoro-1H-benzotriazol-4-yl)-8a,9,11,12-tetrahydropyrazino[2′,1′:3,4][1,4]oxazepino[5,6,7-de]quinazolin-10(8H)-yl]prop-2-en-1-one;
8-[(8aS)-6-Chloro-4-fluoro-10-(prop-2-enoyl)-8,8a,9,10,11,12-hexahydropyrazino[2′,1′:3,4][1,4]oxazepino[5,6,7-de]quinazolin-5-yl]-7-fluoroisoquinolin-1(2H)-one;
(2E)-1-[(8aS)-6-chloro-4-fluoro-5-(5-methyl-1H-indazol-4-yl)-8a,9,11,12-tetrahydropyrazino [2′,1′:3,4][1,4]oxazepino[5,6,7-de]quinazolin-10(8H)-yl]-4-(dimethylamino)but-2-en-1-one;
1-[(6aR,9S)-3-Chloro-1-fluoro-2-(2-fluoro-6-hydroxyphenyl)-9-methyl-5,6,6a,7,9,10-hexahydro-8H-pyrazino[1′,2′:5,6][1,5]oxazocino[4,3,2-de]quinazolin-8-yl]prop-2-en-1-one;
1-[(6aR,9S)-3-Chloro-1-fluoro-2-(2-fluoro-6-hydroxyphenyl)-9-methyl-5,6,6a,7,9,10-hexahydro-8H-pyrazino[1′,2′:5,6][1,5]oxazocino[4,3,2-de]quinazolin-8-yl]prop-2-en-1-one;
8-[3-Chloro-1-fluoro-8-(prop-2-enoyl)-6,6a,7,8,9,10-hexahydro-5H-pyrazino[1′,2′:5,6][1,5]oxazocino[4,3,2-de]quinazolin-2-yl]-7-methylisoquinolin-1(2H)-one;
1-[(6aS,9R)-3-Chloro-1-fluoro-2-(2-fluoro-6-hydroxyphenyl)-9-methyl-5,6,6a,7,9,10-hexahydro-8H-pyrazino[1′,2′:5,6][1,5]oxazocino[4,3,2-de]quinazolin-8-yl]prop-2-en-1-one;
1-[(6aR,9R)-3-Chloro-1-fluoro-2-(2-fluoro-6-hydroxyphenyl)-9-methyl-5,6,6a,7,9,10-hexahydro-8H-pyrazino[1′,2′:5,6][1,5]oxazocino[4,3,2-de]quinazolin-8-yl]prop-2-en-1-one;
1-[(6aS,9S)-3-Chloro-1-fluoro-2-(2-fluoro-6-hydroxyphenyl)-9-methyl-5,6,6a,7,9,10-hexahydro-8H-pyrazino[1′,2′:5,6][1,5]oxazocino[4,3,2-de]quinazolin-8-yl]prop-2-en-1-one;
1-[(8aS)-4-Chloro-6-fluoro-5-(2-fluoro-6-hydroxyphenyl)-8a,9,11,12-tetrahydropyrazino[2′,1′:3,4][1,4]oxazepino[5,6,7-de]quinazolin-10(8H)-yl]prop-2-en-1-one;
1-[(8aS,11R)-6-Chloro-4-fluoro-11-methyl-5-(5-methyl-1H-benzimidazol-4-yl)-8a,9,11,12-tetrahydropyrazino[2′,1′:3,4][1,4]oxazepino[5,6,7-de]quinazolin-10(8H)-yl]prop-2-en-1-one;
8-[(8aS,11R)-6-Chloro-4-fluoro-11-methyl-10-(prop-2-enoyl)-8,8a,9,10,11,12-hexahydropyrazino [2′,1′:3,4][1,4]oxazepino[5,6,7-de]quinazolin-5-yl]-7-methylisoquinolin-1(2H)-one;
(2E)-1-[(8aS,11R)-6-Chloro-4-fluoro-11-methyl-5-(5-methyl-1H-benzimidazol-4-yl)-8a,9,11,12-tetrahydropyrazino[2′,1′:3,4][1,4]oxazepino[5,6,7-de]quinazolin-10(8H)-yl]-4-(dimethylamino)but-2-en-1-one; and
(2E)-1-[(8aS,11R)-6-Chloro-4-fluoro-11-methyl-5-(5-methyl-1H-indazol-4-yl)-8a,9,11,12-tetrahydropyrazino[2′,1′:3,4][1,4]oxazepino[5,6,7-de]quinazolin-10(8H)-yl]-4-(dimethylamino)but-2-en-1-one;
or a pharmaceutically acceptable salt thereof.

In some embodiments, the small molecule KRAS inhibitor is a compound of Formula (II):

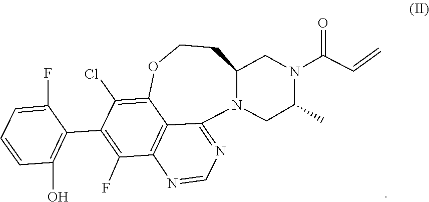 [SMILES: C=CC(=O)N1C[C@@H]2CCOc3c(Cl)c(-c4c(O)cccc4F)c(F)c4ncnc(c34)N2C[C@H]1C]

The compound of Formula (II) is 1-[(6aS,9R)-3-chloro-1-fluoro-2-(2-fluoro-6-hydroxyphenyl)-9-methyl-5,6,6a,7,9,10-hexahydro-8H-pyrazino[1′,2′:5,6][1,5]oxazocino[4,3,2-de]quinazolin-8-yl]prop-2-en-1-one, or a pharmaceutically acceptable salt thereof.

In some embodiments, the small molecule KRAS inhibitor is a compound of Formula (III):

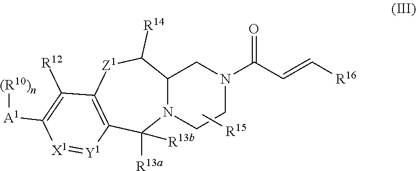

as defined herein.

In some embodiments of Formula (III), i) X¹ is CR¹⁷ and Y¹ is CR⁸, ii) X¹ is N and Y¹ is CR¹⁸ or iii) X¹ is CR¹⁷ and Y¹ is N. In some embodiments of Formula (III), Z¹ is O. In some embodiments of Formula (III), R^{13a} and R^{13b} are H. In some embodiments of Formula (III), R¹⁴ is H. In some embodiments of Formula (III), R¹⁶ is H. In some embodiments of Formula (III), A¹ is phenyl. In some embodiments, the small molecule KRAS inhibitor is selected from:
(12aS)-2-Acryloyl-10-chloro-9-(5-methyl-1H-indazol-4-yl)-1,2,3,4,12,12a-hexahydro-6H-benzo[f]pyrazino[2,1-c][1,4]oxazepin-6-one;
1-((12aS)-10-Chloro-9-(5-methyl-1H-indazol-4-yl)-3,4,12,12a-tetrahydro-6H-benzo[f]pyrazino[2,1-c][1,4]oxazepin-2(1H)-yl)prop-2-en-1-one;
1-[(12aR)-10-Chloro-9-(2-fluoro-6-hydroxyphenyl)-7-methoxy-3,4,12,12a-tetrahydro-6H-pyrazino[2,1-c][1,4]benzoxazepin-2(1H)-yl]prop-2-en-1-one;
(12aR)-10-chloro-9-(2-fluoro-6-hydroxyphenyl)-7-hydroxy-2-(prop-2-enoyl)-1,2,3,4,12,12a-hexahydro-6H-pyrazino[2,1-c][1,4]benzoxazepin-6-one;
(12aR)-10-Chloro-9-(5-methyl-1H-indazol-4-yl)-2-(prop-2-enoyl)-1,2,3,4,12,12a-hexahydro-6H-pyrazino[2,1-c][1,4]benzoxazepin-6-one;
1-((12aR)-10-Chloro-9-(5-methyl-1H-indazol-4-yl)-3,4,12,12a-tetrahydro-6H-benzo[f]pyrazino[2,1-c][1,4]oxazepin-2(1H)-yl)prop-2-en-1-one;
(12aR)-10-Chloro-8-fluoro-9-(2-fluoro-6-hydroxyphenyl)-2-(prop-2-enoyl)-1,2,3,4,12,12a-hexahydro-6H-pyrazino[2,1-c][1,4]benzoxazepin-6-one;
1-[(12aR)-8,10-dichloro-9-(2-fluoro-6-hydroxyphenyl)-7-hydroxy-3,4,12,12a-tetrahydro-6H-pyrazino[2,1-c][1,4]benzoxazepin-2(1H)-yl]prop-2-en-1-one;
1-[(12aR)-10-Chloro-9-(2-fluoro-6-hydroxyphenyl)-7-(1H-imidazol-1-yl)-3,4,12,12a-tetrahydro-6H-pyrazino[2,1-c][1,4]benzoxazepin-2(1H)-yl]prop-2-en-1-one;
(12aR)-10-Chloro-9-(2-fluoro-6-hydroxyphenyl)-2-(prop-2-enoyl)-1,2,3,4,12,12a-hexahydro-6H-pyrazino[2,1-c][1,4]benzoxazepine-7-carbonitrile;
1-[(12aR)-10-Chloro-9-(2-fluoro-6-hydroxyphenyl)-7-(1H-pyrazol-1-yl)-3,4,12,12a-tetrahydro-6H-pyrazino[2,1-c][1,4]benzoxazepin-2(1H)-yl]prop-2-en-1-one;
1-((12aR)-10-Chloro-8-fluoro-9-(5-methyl-1H-benzo[d]imidazol-4-yl)-3,4,12,12a-tetrahydro-6H-benzo[f]pyrazino[2,1-c][1,4]oxazepin-2(1H)-yl)prop-2-en-1-one;
(12aR)-8,10-Dichloro-9-(2-fluoro-6-hydroxyphenyl)-2-(prop-2-enoyl)-1,2,3,4,12,12a-hexahydro-6H-pyrazino[2,1-c][1,4]benzoxazepin-6-one;
(12aR)-10-Chloro-9-(2-fluoro-6-hydroxyphenyl)-2-(prop-2-enoyl)-1,2,3,4,12,12a-hexahydro-6H-pyrazino[2,1-c][1,4]benzoxazepine-8-carbonitrile;
(12aR)-10-Chloro-9-(2-fluoro-6-hydroxyphenyl)-8-methyl-2-(prop-2-enoyl)-1,2,3,4,12,12a-hexahydro-6H-pyrazino[2,1-c][1,4]benzoxazepin-6-one;
1-[(12aR)-8,10-Dichloro-9-(2-fluoro-6-hydroxyphenyl)-3,4,12,12a-tetrahydro-6H-pyrazino[2,1-c][1,4]benzoxazepin-2(1H)-yl]prop-2-en-1-one;
8-[(12aR)-10-Chloro-8-fluoro-2-(prop-2-enoyl)-1,2,3,4,12,12a-hexahydro-6H-pyrazino[2,1-c][1,4]benzoxazepin-9-yl]-7-methylisoquinolin-1(2H)-one;
1-[(12aR)-10-chloro-9-(2-fluoro-6-hydroxyphenyl)-8-methoxy-3,4,12,12a-tetrahydro-6H-pyrazino[2,1-c][1,4]benzoxazepin-2(1H)-yl]prop-2-en-1-one;
(12aS)-10-Chloro-9-(5-methyl-1H-indazol-4-yl)-2-(prop-2-enoyl)-1,3,4,11,12,12a-hexahydropyrazino[2,1-c][1,4]benzodiazepin-6(2H)-one;
1-[(12aR)-10-chloro-9-(2-chloro-6-hydroxyphenyl)-8-fluoro-3,4,12,12a-tetrahydro-6H-pyrazino[2,1-c][1,4]benzoxazepin-2(1H)-yl]prop-2-en-1-one;
(12aS)-10-Chloro-11-methyl-9-(5-methyl-1H-indazol-4-yl)-2-(prop-2-enoyl)-1,3,4,11,12,12a-hexahydropyrazino[2,1-c][1,4]benzodiazepin-6(2H)-one;
1-[(12aR)-10-Chloro-9-(2,3-difluoro-6-hydroxyphenyl)-8-fluoro-3,4,12,12a-tetrahydro-6H-pyrazino[2,1-c][1,4]benzoxazepin-2(1H)-yl]prop-2-en-1-one;
(12aR)-10-Chloro-9-(2-hydroxy-6-methylphenyl)-2-(prop-2-enoyl)-1,2,3,4,12,12a-hexahydro-6H-pyrazino[2,1-c][1,4]benzoxazepine-8-carbonitrile;
1-[(12aR)-9-(2-Chloro-6-hydroxyphenyl)-8,10-difluoro-3,4,12,12a-tetrahydro-6H-pyrazino[2,1-c][1,4]benzoxazepin-2(1H)-yl]prop-2-en-1-one;
1-[(12aR)-8,10-Difluoro-9-(2-hydroxy-6-methylphenyl)-3,4,12,12a-tetrahydro-6H-pyrazino[2,1-c][1,4]benzoxazepin-2(1H)-yl]prop-2-en-1-one;
1-[(12aR)-8,10-Difluoro-9-[2-fluoro-6-(hydroxymethyl)phenyl]-3,4,12,12a-tetrahydro-6H-pyrazino[2,1-c][1,4]benzoxazepin-2(1H)-yl]prop-2-en-1-one;
1-[(12aR)-8,10-Difluoro-9-[2-hydroxy-6-(trifluoromethyl)phenyl]-3,4,12,12a-tetrahydro-6H-pyrazino[2,1-c][1,4]benzoxazepin-2(1H)-yl]prop-2-en-1-one;
1-[(12aR)-9-(2-Ethyl-6-hydroxyphenyl)-8,10-difluoro-3,4,12,12a-tetrahydro-6H-pyrazino[2,1-c][1,4]benzoxazepin-2(1H)-yl]prop-2-en-1-one;
1-[(12aR)-9-[2-(Difluoromethyl)-6-hydroxyphenyl]-8,10-difluoro-3,4,12,12a-tetrahydro-6H-pyrazino[2,1-c][1,4]benzoxazepin-2(1H)-yl]prop-2-en-1-one;
(12aR)-9-(2-Chloro-6-hydroxyphenyl)-10-fluoro-2-(prop-2-enoyl)-1,2,3,4,12,12a-hexahydro-6H-pyrazino[2,1-c][1,4]benzoxazepine-8-carbonitrile;
(12aR)-10-Chloro-9-(2-chloro-6-hydroxyphenyl)-2-(prop-2-enoyl)-1,2,3,4,12,12a-hexahydro-6H-pyrazino[2,1-c][1,4]benzoxazepine-8-carbonitrile;
1-[(12aR)-9-(2-Bromo-6-hydroxyphenyl)-8,10-difluoro-3,4,12,12a-tetrahydro-6H-pyrazino[2,1-c][1,4]benzoxazepin-2(1H)-yl]prop-2-en-1-one;
1-[(12aR)-8-Chloro-10-fluoro-9-(2-hydroxy-6-methylphenyl)-3,4,12,12a-tetrahydro-6H-pyrazino[2,1-c][1,4]benzoxazepin-2(1H)-yl]prop-2-en-1-one;
1-[(12aR)-8-Chloro-10-ethynyl-9-(2-hydroxy-6-methylphenyl)-3,4,12,12a-tetrahydro-6H-pyrazino[2,1-c][1,4]benzoxazepin-2(1H)-yl]prop-2-en-1-one;
1-[(12aR)-10-Ethynyl-8-fluoro-9-(2-hydroxy-6-methylphenyl)-3,4,12,12a-tetrahydro-6H-pyrazino[2,1-c][1,4]benzoxazepin-2(1H)-yl]prop-2-en-1-one;
(6aR)-4-Chloro-3-(2-fluoro-6-hydroxyphenyl)-2-methyl-8-(prop-2-enoyl)-2,6,6a,7,8,9,10,12-octahydro-1H-pyrazino[2,1-c]pyrido[3,4-f][1,4]oxazepin-1-one;
1-[(6aR)-1,4-Dichloro-3-(2-fluoro-6-hydroxyphenyl)-6a,7,9,10-tetrahydro-12H-pyrazino[2,1-c]pyrido[3,4-J][1,4]oxazepin-8(6H)-yl]prop-2-en-1-one;
(6aR)-4-Chloro-3-(2-fluoro-6-hydroxyphenyl)-8-(prop-2-enoyl)-2,6,6a,7,8,9,10,12-octahydro-1H-pyrazino[2,1-c]pyrido[3,4-f][1,4]oxazepin-1-one;
1-[(8aR)-6-Chloro-5-(2-fluoro-6-hydroxyphenyl)-8a,9,11,12-tetrahydro-14H-pyrazino[2,1-c][1,2,4]triazolo[4′,3′:1,2]pyrido[3,4-f][1,4]oxazepin-10(8H)-yl]prop-2-en-1-one;
1-[(7aR)-5-chloro-4-(2-fluoro-6-hydroxyphenyl)-1-methyl-1,7a,8,10,11,13-hexahydropyrazino[2′,1′:3,4][1,4]oxazepino[7,6-g]indazol-9(7H)-yl]prop-2-en-1-one; and
1-[(7aR)-5-Chloro-4-(2-fluoro-6-hydroxyphenyl)-2-methyl-2,7a,8,10,11,13-hexahydropyrazino[2′,1′:3,4][1,4]oxazepino[7,6-g]indazol-9(7H)-yl]prop-2-en-1-one;
1-[(12aR)-9-(2-chloro-6-hydroxyphenyl)-8-ethynyl-10-fluoro-3,4,12,12a-tetrahydro-6H-pyrazino[2,1-c][1,4]benzoxazepin-2(1H)-yl]prop-2-en-1-one;
1-[(12aR)-10-Chloro-9-(2-chloro-6-hydroxyphenyl)-8-ethynyl-3,4,12,12a-tetrahydro-6H-pyrazino[2,1-c][1,4]benzoxazepin-2(1H)-yl]prop-2-en-1-one;
1-((12aR)-10-chloro-8-ethynyl-9-(2-fluoro-6-hydroxyphenyl)-3,4,12,12a-tetrahydro-6H-benzo[f]pyrazino[2,1-c][1,4]oxazepin-2(1H)-yl)prop-2-en-1-one;
1-[(7aR)-5-Chloro-4-(2-chloro-6-hydroxyphenyl)-1-methyl-1,7a,8,10,11,13-hexahydroimidazo[4,5-g]pyrazino[2,1-c][1,4]benzoxazepin-9(7H)-yl]prop-2-en-1-one;
1-[(12aR)-8-Chloro-9-(2-chloro-6-hydroxyphenyl)-10-fluoro-3,4,12,12a-tetrahydro-6H-pyrazino[2,1-c][1,4]benzoxazepin-2(1H)-yl]prop-2-en-1-one;
1-[(12aR)-9-(2-Chloro-6-hydroxyphenyl)-10-fluoro-8-(prop-1-yn-1-yl)-3,4,12,12a-tetrahydro-6H-pyrazino[2,1-c][1,4]benzoxazepin-2(1H)-yl]prop-2-en-1-one;
1-[(6aR)-4-Chloro-3-(2-chloro-6-hydroxyphenyl)-2-ethynyl-6a,7,9,10-tetrahydro-12H-pyrazino[2,1-c]pyrido[2,3-][1,4]oxazepin-8(6H)-yl]prop-2-en-1-one;
1-[(12aR)-9-(2-Chloro-6-hydroxyphenyl)-8-ethynyl-10-methyl-3,4,12,12a-tetrahydro-6H-pyrazino[2,1-c][1,4]benzoxazepin-2(1H)-yl]prop-2-en-1-one;
1-[(12aR)-10-chloro-9-(2-chloro-6-hydroxyphenyl)-7,8-difluoro-3,4,12,12a-tetrahydro-6H-pyrazino[2,1-c][1,4]benzoxazepin-2(1H)-yl]prop-2-en-1-one;
1-[(12aR)-9-(2-chloro-6-hydroxyphenyl)-8-(difluoromethoxy)-10-fluoro-3,4,12,12a-tetrahydro-6H-pyrazino[2,1-c][1,4]benzoxazepin-2(1H)-yl]prop-2-en-1-one;
1-[(12aR)-9-(2-chloro-6-hydroxyphenyl)-8-ethynyl-7,10-difluoro-3,4,12,12a-tetrahydro-6H-pyrazino[2,1-c][1,4]benzoxazepin-2(1H)-yl]prop-2-en-1-one;
1-[(12aR)-10-Chloro-9-(2-chloro-6-hydroxyphenyl)-8-(difluoromethoxy)-3,4,12,12a-tetrahydro-6H-pyrazino[2,1-c][1,4]benzoxazepin-2(1H)-yl]prop-2-en-1-one;
1-[(12aR)-9-(2-chloro-6-hydroxyphenyl)-8-(cyclopropyloxy)-10-fluoro-3,4,12,12a-tetrahydro-6H-pyrazino[2,1-c][1,4]benzoxazepin-2(1H)-yl]prop-2-en-1-one;
1-((12aR)-9-(2-Chloro-6-hydroxyphenyl)-8-(3-(dimethylamino)prop-1-yn-1-yl)-10-fluoro-3,4,12,12a-tetrahydro-6H-benzo[f]pyrazino[2,1-c][1,4]oxazepin-2(1H)-yl)prop-2-en-1-one;
1-[(12aR)-9-(2-chloro-6-hydroxyphenyl)-10-fluoro-8-[(pyridin-4-yl)methoxy]-3,4,12,12a-tetrahydro-6H-pyrazino[2,1-c][1,4]benzoxazepin-2(1H)-yl]prop-2-en-1-one;
1-[(12aR)-10-chloro-9-(2-chloro-6-hydroxyphenyl)-8-(2-methoxyethoxy)-3,4,12,12a-tetrahydro-6H-pyrazino[2,1-c][1,4]benzoxazepin-2(1H)-yl]prop-2-en-1-one;
1-[(12aR)-9-(2-Chloro-6-hydroxyphenyl)-10-fluoro-8-[2-(piperidin-1-yl)ethoxy]-3,4,12,12a-tetrahydro-6H-pyrazino[2,1-c][1,4]benzoxazepin-2(1H)-yl]prop-2-en-1-one;
1-[(12aR)-10-Chloro-9-(2-chloro-6-hydroxyphenyl)-8-(prop-1-yn-1-yl)-3,4,12,12a-tetrahydro-6H-pyrazino[2,1-c][1,4]benzoxazepin-2(1H)-yl]prop-2-en-1-one;
1-[(6aR)-4-Chloro-3-(2-chloro-6-hydroxyphenyl)-2-[(²H₃)methyloxy]-6a,7,9,10-tetrahydro-12H-pyrazino[2,1-c]pyrido[2,3-][1,4]oxazepin-8(6H)-yl]prop-2-en-1-one;
1-[(12aR)-10-Chloro-9-(2-chloro-6-hydroxyphenyl)-8-(methoxymethyl)-3,4,12,12a-tetrahydro-6H-pyrazino[2,1-c][1,4]benzoxazepin-2(1H)-yl]prop-2-en-1-one; and
1-[(12aR)-9-(2-Chloro-6-hydroxyphenyl)-7-[2-(dimethylamino)ethoxy]-10-fluoro-3,4,12,12a-tetrahydro-6H-pyrazino[2,1-c][1,4]benzoxazepin-2(1H)-yl]prop-2-en-1-one;
1-[(6aR)-4-Chloro-3-(2-chloro-6-hydroxyphenyl)-1-(prop-1-yn-1-yl)-6a,7,9,10-tetrahydro-12H-pyrazino[2,1-c]pyrido[3,4-f][1,4]oxazepin-8(6H)-yl]prop-2-en-1-one; and
1-((6aR)-4-Chloro-3-(2-chloro-6-hydroxyphenyl)-1-ethynyl-6a,7,9,10-tetrahydro-12H-pyrazino[2,1-c]pyrido[3,4-f][1,4]oxazepin-8(6H)-yl)prop-2-en-1-one;
or a pharmaceutically acceptable salt thereof.

In some embodiments, the small molecule KRAS inhibitor is a compound of Formula (IV):

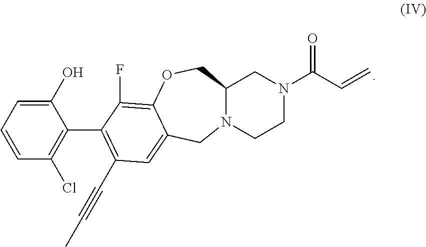 [SMILES: C=CC(=O)N1CCN2Cc3cc(C#CC)c(-c4c(O)cccc4Cl)c(F)c3OC[C@H]2C1]

The compound of Formula (IV) is 1-[(12aR)-9-(2-chloro-6-hydroxyphenyl)-10-fluoro-8-(prop-1-yn-1-yl)-3,4,12,12a-tetrahydro-6H-pyrazino[2,1-c][1,4]benzoxazepin-2(1H)-yl]prop-2-en-1-one, or a pharmaceutically acceptable salt thereof.

In some embodiments, the ATR inhibitor is an antibody. In some embodiments, the ATR inhibitor is a small molecule ATR inhibitor. In some embodiments, the small molecule ATR inhibitor is M6620/VX970 (berzosertib), BAY-1895344 (elimusertib), RP-3500 (camonsertib), ATRN-119, SC0245, ATRN-212, LR-02, M4344 (Gartisertib), M1774, ATG-018, ARTO380, BG-129, JS-123, BKT-300, AZ-20, VE-821, AZD-5597 or IMP9064. In some embodiments, the small molecule ATR inhibitor is a compound of Formula (V):

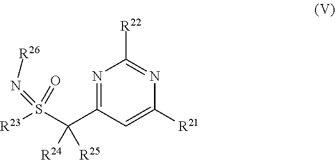

as defined herein.

In some embodiments, R²⁴ and R²⁵ together with the atom to which they are attached form Ring A², and Ring A² is a C₃₋₆ cycloalkyl or a saturated 4-6 heterocyclic ring containing one heteroatom selected from O and N. In some embodiments, Ring A² is a cyclopropyl, tetrahydropyranyl or piperidinyl ring. In some embodiments, R^{22A} is hydrogen; R^{22B} is hydrogen; R^{22C} is hydrogen; R^{22D} is hydrogen; R^{22E} is hydrogen; and R^{22F} is hydrogen. In some embodiments, R²¹ is morpholin-4-yl. In some embodiments, R²¹ is 3-methylmorpholin-4-yl. In some embodiments, the compound of Formula (V) is a compound of Formula (Va),

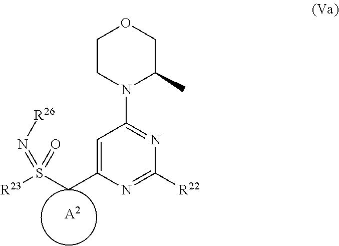

as defined herein. In some embodiments, the small molecule ATR inhibitor is selected from any one of:
4-{4-[(3R)-3-Methylmorpholin-4-yl]-6-[((R)—S-methylsulfonimidoyl)methyl]pyrimidin-2-yl}-1H-pyrrolo[2,3-b]pyridine;
4-{4-[(3R)-3-Methylmorpholin-4-yl]-6-[1-((S)—S-methylsulfonimidoyl)cyclopropyl]pyrimidin-2-yl}-1H-pyrrolo[2,3-b]pyridine;
4-{4-[(3R)-3-Methylmorpholin-4-yl]-6-[1-((R)—S-methylsulfonimidoyl)cyclopropyl]pyrimidin-2-yl}-1H-pyrrolo[2,3-b]pyridine;
N-Methyl-1-{4-[(3R)-3-methylmorpholin-4-yl]-6-[1-((R)—S-methylsulfonimidoyl)cyclopropyl]pyrimidin-2-yl}-1H-benzimidazol-2-amine;
N-Methyl-1-{4-[(3R)-3-methylmorpholin-4-yl]-6-[1-((S)—S-methylsulfonimidoyl)cyclopropyl]pyrimidin-2-yl}-1H-benzimidazol-2-amine;
4-{4-[(3R)-3-Methylmorpholin-4-yl]-6-[1-((R)—S-methylsulfonimidoyl)cyclopropyl]pyrimidin-2-yl}-1H-indole;
4-{4-[(3R)-3-methylmorpholin-4-yl]-6-[1-((S)—S-methylsulfonimidoyl)cyclopropyl]pyrimidin-2-yl}-1H-indole;
1-{4-[(3R)-3-Methylmorpholin-4-yl]-6-[1-((R)—S-methylsulfonimidoyl)cyclopropyl]pyrimidin-2-yl}-1H-benzimidazol-2-amine;
1-{4-[(3R)-3-methylmorpholin-4-yl]-6-[1-((S)—S-methylsulfonimidoyl)cyclopropyl]pyrimidin-2-yl}-1H-benzimidazol-2-amine;
4-Fluoro-N-methyl-1-{4-[(3R)-3-methylmorpholin-4-yl]-6-[1-((R)—S-methylsulfonimidoyl)cyclopropyl]pyrimidin-2-yl}-1H-benzimidazol-2-amine;
4-fluoro-N-methyl-1-{4-[(3R)-3-methylmorpholin-4-yl]-6-[1-((S)—S-methylsulfonimidoyl)cyclopropyl]pyrimidin-2-yl}-1H-benzimidazol-2-amine;
4-{4-[(3R)-3-Methylmorpholin-4-yl]-6-[1-(S-methylsulfonimidoyl)cyclopropyl]pyrimidin-2-yl}-1H-pyrrolo[2,3-c]pyridine;
N-methyl-1-{4-[1-methyl-1-((S)—S-methylsulfonimidoyl)ethyl]-6-[(3R)-3-methylmorpholin-4-yl]pyrimidin-2-yl}-1H-benzimidazol-2-amine;
N-methyl-1-{4-[1-methyl-1-((R)—S-methylsulfonimidoyl)ethyl]-6-[(3R)-3-methylmorpholin-4-yl]pyrimidin-2-yl}-1H-benzimidazol-2-amine;
N-Methyl-1-{4-[(3R)-3-methylmorpholin-4-yl]-6-[4-((S)—S-methylsulfonimidoyl)tetrahydro-2H-pyran-4-yl]pyrimidin-2-yl}-1H-benzimidazol-2-amine;
N-methyl-1-{4-[(3R)-3-methylmorpholin-4-yl]-6-[4-((R)—S-methylsulfonimidoyl)tetrahydro-2H-pyran-4-yl]pyrimidin-2-yl}-1H-benzimidazol-2-amine;
4-{4-[(3R)-3-Methylmorpholin-4-yl]-6-[4-((S)—S-methylsulfonimidoyl)tetrahydro-2H-pyran-4-yl]pyrimidin-2-yl}-1H-indole;
4-Fluoro-N-methyl-1-{4-[1-methyl-1-((S)—S-methylsulfonimidoyl)ethyl]-6-[(3R)-3-methylmorpholin-4-yl]pyrimidin-2-yl}-1H-benzimidazol-2-amine;
4-fluoro-N-methyl-1-{4-[i-methyl-1-((R)—S-methylsulfonimidoyl)ethyl]-6-[(3R)-3-methylmorpholin-4-yl]pyrimidin-2-yl}-1H-benzimidazol-2-amine;
6-Fluoro-N-methyl-1-{4-[1-methyl-1-((R)—S-methylsulfonimidoyl)ethyl]-6-[(3R)-3-methylmorpholin-4-yl]pyrimidin-2-yl}-1H-benzimidazol-2-amine;
5-Fluoro-N-methyl-1-{4-[1-methyl-1-((R)—S-methylsulfonimidoyl)ethyl]-6-[(3R)-3-methylmorpholin-4-yl]pyrimidin-2-yl}-1H-benzimidazol-2-amine;
5-Fluoro-N-methyl-1-{4-[1-methyl-1-((S)—S-methylsulfonimidoyl)ethyl]-6-[(3R)-3-methylmorpholin-4-yl]pyrimidin-2-yl}-1H-benzimidazol-2-amine;
6-fluoro-N-methyl-1-{4-[1-methyl-1-((S)—S-methylsulfonimidoyl)ethyl]-6-[(3R)-3-methylmorpholin-4-yl]pyrimidin-2-yl}-1H-benzimidazol-2-amine;
6-Fluoro-N-methyl-1-{4-[(3R)-3-methylmorpholin-4-yl]-6-[1-((R)—S-methylsulfonimidoyl)cyclopropyl]pyrimidin-2-yl}-1H-benzimidazol-2-amine;
5-fluoro-N-methyl-1-{4-[(3R)-3-methylmorpholin-4-yl]-6-[1-((R)—S-methylsulfonimidoyl)cyclopropyl]pyrimidin-2-yl}-1H-benzimidazol-2-amine;
5-fluoro-N-methyl-1-{4-[(3R)-3-methylmorpholin-4-yl]-6-[1-((S)—S-methylsulfonimidoyl)cyclopropyl]pyrimidin-2-yl}-1H-benzimidazol-2-amine; and
6-fluoro-N-methyl-1-{4-[(3R)-3-methylmorpholin-4-yl]-6-[1-((S)—S-methylsulfonimidoyl)cyclopropyl]pyrimidin-2-yl}-1H-benzimidazol-2-amine,
or a pharmaceutically acceptable salt thereof.

In some embodiments, the small molecule ATR inhibitor is a compound of Formula (VI) (ceralasertib):

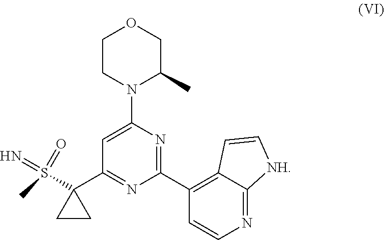 [SMILES: C[C@@H]1COCCN1c1cc(C2([S@](C)(=N)=O)CC2)nc(-c2ccnc3[nH]ccc23)n1]

The compound of Formula (VI) is 4-{4-[(3R)-3-methylmorpholin-4-yl]-6-[1-((R)—S-methylsulfonimidoyl)cyclopropyl]pyrimidin-2-yl}-1H-pyrrolo[2,3-b]pyridine, or a pharmaceutically acceptable salt thereof.

In some embodiments, the (i) KRAS inhibitor, and (ii) ATR inhibitor is one of the combinations in Table 4. In some embodiments, the (i) KRAS inhibitor is a compound of Formula (II), and (ii) ATR inhibitor is ceralasertib. In some embodiments, the (i) KRAS inhibitor is a compound of Formula (IV), and (ii) ATR inhibitor is ceralasertib.

In some embodiments, the subject has a KRAS, NRAS or HRAS mutation. In some embodiments, the subject has a KRAS mutation. In some embodiments, the KRAS mutation is a mutation at codon 12, codon 13 and/or codon 61. In some embodiments, the KRAS mutation is a mutation at codon 12. In some embodiments, the KRAS mutation is a G12C mutation. In some embodiments, the subject has lung cancer, colorectal cancer, pancreatic cancer or combination thereof. In some embodiments, the subject is a human subject. In some embodiments, the subject has lung cancer, colorectal cancer, pancreatic cancer, esophagogastric cancer, endometrial cancer, cholangiocarcinoma or combination thereof.

In some embodiments, the disclosure provides a method of modulating the adaptive immune response in a subject, the method comprising administering a composition comprising: (a) a KRAS inhibitor, and (b) an ATR inhibitor. In some embodiments, the disclosure provides a method of reducing the volume of a cancerous tumor in a subject, the method comprising administering a composition comprising: (a) a KRAS inhibitor, and (b) an ATR inhibitor. In some embodiments, the disclosure provides a method of treating a subject in need thereof, the method comprising administering a composition comprising: (a) a KRAS inhibitor, and (b) an ATR inhibitor, wherein the subject has a disorder mediated by a KRAS, NRAS or HRAS G12C mutation.

The disclosure also provides for a composition comprising: (a) a KRAS inhibitor, and (b) an ATR inhibitor. In some embodiments, the KRAS inhibitor is sotorasib, adagrasib, Ly3537982, GDC-6036, D-1553, JDQ443, BI1823911, GFH925, D35-001, JAB-21822, HBI-2438, YL-15293, GEC255, IBI351, RMC-6291, MK-1084, MRTX1133, HRS-4642, RMC-9805, JAB-2200, Vrtx153, ERAS-4, ASP3082, RMC-0708, RMC-8839, RMC-6291, JAB-23400, TEB-17231, QTX3046, a compound of Formula (I), a compound of Formula (II), a compound of Formula (III), or a compound of Formula (IV). In some embodiments, the ATR inhibitor is M6620/VX970 (berzosertib), BAY-1895344 (elimusertib), RP-3500 (camonsertib), ATRN-119, SC0245, ATRN-212, LR-02, M4344 (Gartisertib), M1774, ATG-018, ARTO380, BG-129, JS-123, BKT-300, AZ-20, VE-821, AZD-5597, or IMP9064, a compound of Formula (V), or a compound of Formula (VI).

In some embodiments, the composition is a liquid. In some embodiments, the composition is a solid. In some embodiments, the composition is a solid oral dosage form. In some embodiments, the composition further comprises a pharmaceutically acceptable excipient, diluent or carrier.

The disclosure also provides for use of the composition as described herein, for the manufacture of a medicament, optionally a medicament for the treatment of cancer.

In some embodiments, the disclosure provides a kit comprising: (a) a first container comprising a first composition comprising a KRAS inhibitor, and (b) a second container comprising a second composition comprising an ATR inhibitor. In some embodiments, the KRAS inhibitor in the kit is sotorasib, adagrasib, Ly3537982, GDC-6036, D-1553, JDQ443, BI11823911, GFH925, D35-001, JAB-21822, HBI-2438, YL-15293, GEC255, IBI351, RMC-6291, MK-1084, MRTX1133, HRS-4642, RMC-9805, JAB-2200, Vrtx153, ERAS-4, ASP3082, RMC-0708, RMC-8839, RMC-6291, JAB-23400, TEB-17231, QTX3046, a compound of Formula (I), a compound of Formula (II), a compound of Formula (III), or a compound of Formula (IV). In some embodiments, the ATR inhibitor in the kit is M6620/VX970 (berzosertib), BAY-1895344 (elimusertib), RP-3500 (camonsertib), ATRN-119, SC0245, ATRN-212, LR-02, M4344 (Gartisertib), M1774, ATG-018, ARTO380, BG-129, JS-123, BKT-300, AZ-20, VE-821, AZD-5597, or IMP9064, a compound of Formula (V), or a compound of Formula (VI).

In some embodiments, the first composition, the second composition, or both the first composition and the second composition is/are a liquid. In some embodiments, the first composition, the second composition, or both the first composition and the second composition is/are a solid. In some embodiments, the first composition, the second composition, or both the first composition and the second composition is/are a solid oral dosage form. In some embodiments, the first composition, the second composition, or both the first composition and the second composition further comprise a pharmaceutically acceptable excipient, diluent or carrier.

### BRIEF DESCRIPTION OF THE FIGURES

FIG. 1. Inhibition of KRAS and ATR drive tumor regressions in KRAS^{G12C} mutant models. FIG. 1A: Schematic showing the dosing schedule of AZD4625 (Compound of Formula II) and AZD6738 (ceralasertib) used in the study. Tumor growth (FIG. 1B) or bodyweight changes (FIG. 1C) in individual animals dosed with AZD4625 or AZD6738 in monotherapy or combination or controls.

FIG. 2. Combination activity expected with all allosteric G12C inhibitors. FIG. 2A: Schematic showing the dosing schedule of AZD4625, Compound of Formula IV, and AZD6738 used in the study. FIG. 2B Tumor growth in individual animals dosed with AZD4625, Compound of Formula IV, or AZD6738 in monotherapy or combination or controls.

FIG. 3. KRAS and ATR inhibition provides long term anti-tumor protection.

FIG. 3A: Schematic of rechallenge study design. FIG. 3B: Individual CT26^{G12C} tumor growth in the left flank of naïve mice or mice rechallenged following complete anti-tumor response to Compound of Formula IV and AZD6738.

FIG. 4. Durable anti-tumor responses are achieved in preclinical models with an intact immune system. Comparison of monotherapy and combination responses with KRAS^{G12C} inhibitors (either AZD4625 or Compound of Formula (IV)) and AZD6738 in immunodeficient Nude and immunocompetent BALB/c mice on tumor growth during the 4-week drug treatment period (FIG. 4A) or time to reach endpoint (FIG. 4B). Grey bars in FIG. 4A indicate treatment period for AZD6738. Grey bars in FIG. 4B indicate dosing period for all treatments.

FIG. 5. Durable anti-tumor responses are achieved in preclinical models with an intact immune system. Comparison of monotherapy and combination responses with KRAS^{G12C} inhibitors (either adagrasib or sotorasib) and AZD6738 in immunocompetent BALB/c mice on tumor growth during the 4-week drug treatment period (FIG. 5A), time to reach endpoint (FIG. 5B) and percentage body weight change (FIG. 5C). Grey bars in FIG. 5A and FIG. 5C indicate treatment period for AZD6738. Grey bars in FIG. 5B indicate dosing period for all treatments.

### DETAILED DESCRIPTION

Unless otherwise defined herein, scientific, and technical terms used in the present disclosure shall have the meanings that are commonly understood by one of ordinary skill in the art.

Further, unless otherwise required by context, singular terms shall include pluralities and plural terms shall include the singular. As used herein, “a” or “an” may mean one or more. As used herein, when used in conjunction with the word “comprising,” the words “a” or “an” may mean one or more than one. As used herein, “another” or “a further” may mean at least a second or more.

The use of the term “or” in the claims is used to mean “and/or,” unless explicitly indicated to refer only to alternatives or the alternatives are mutually exclusive, although the disclosure supports a definition that refers to only alternatives and “and/or.”

As used herein, the terms “comprising” (and any variant or form of comprising, such as “comprise” and “comprises”), “having” (and any variant or form of having, such as “have” and “has”), “including” (and any variant or form of including, such as “includes” and “include”) or “containing” (and any variant or form of containing, such as “contains” and “contain”) are inclusive or open-ended and do not exclude additional, unrecited elements or method steps.

Throughout this application, the term “about” is used to indicate that a value includes the inherent variation of error for the method/device being employed to determine the value, or the variation that exists among the study subjects. Typically, the term “about” is meant to encompass approximately or less than 1%, 2%, 3%, 4%, 5%, 6%, 7%, 8%, 9%, 10%, 11%, 12%, 13%, 14%, 15%, 16%, 17%, 18%, 19% or 20% or higher variability (either “greater than” or “less than” the indicated value), depending on the situation. In embodiments, one of skill in the art will understand the level of variability indicated by the term “about,” due to the context in which it is used herein. It should also be understood that use of the term “about” also includes the specifically recited value.

The use of the term “for example” and its corresponding abbreviation “e.g.,” (whether italicized or not) means that the specific terms recited are representative examples and embodiments of the disclosure that are not intended to be limited to the specific examples referenced or cited unless explicitly stated otherwise.

Ranges provided herein, of any type, include all values within a particular range described and values about an endpoint for a particular range. As used herein, “between” is a range inclusive of the ends of the range. For example, a number between x and y explicitly includes the numbers x and y, and any numbers that fall within x and y.

The term “treating” or “treatment” refers to administering a compound or pharmaceutical composition to an animal in order to affect an alteration or improvement of a disease, disorder, or condition in the animal. In some embodiments, the “treating” or “treatment” is provided to a subject suffering from a condition suitable to treatment with the combinations as described herein. In some embodiments, the “treating” or “treatment” is provided to a subject at high risk for suffering from a KRAS and/or ATR dependent condition, as described herein, even before any clinical signs of such condition are manifest in the subject.

The term “administration” or “administering” refers to routes of introducing a compound or composition provided herein to an individual to perform its intended function. An example of a route of administration that can be used includes, but is not limited to, parenteral administration, such as subcutaneous, intravenous, or intramuscular injection or infusion, or oral administration, e.g., with a solid oral dosage form.

The term “subject” is meant to include any subject, particularly a mammalian subject, in need of treatment with the KRAS inhibitor/ATR inhibitor as described herein. Mammalian subjects include human or non-human animal. In some embodiments, the term “subject” refers to a human subject. In some embodiments, the term “subject” refers to a female subject. In some embodiments, the term “subject” refers to a male subject. In some embodiments, the human subject is 12 years of age or older, 14 years of age or older, 4-17 years of age or older, 18 years of age or older. Non-human animal includes, but are not limited to, pigs, dogs, cats, guinea pigs, rabbits, rats, mice, horses, cattle, bears, cows, apes, monkeys, orangutans, and chimpanzees, and so on.

As used herein, a “subject in need thereof” refers to the subject for whom it is desirable to treat, e.g., a subject having a cancer and/or KRAS mutation as described herein. In some embodiments, the term “subject in need thereof” can refer to a subject at high risk for suffering from a cancer and/or KRAS mutation as described herein, independently of whether the subject has physical manifestations of such condition.

“Pharmaceutically acceptable salt” refers to a salt of a compound that is physiologically and pharmaceutically acceptable and that possesses the desired pharmacological activity of the parent compound and includes a salt prepared from pharmaceutically acceptable non-toxic acid or base, including inorganic or organic acids and bases. “Pharmaceutically acceptable salts” of the compounds described herein may be prepared by methods well-known in the art. For a review of pharmaceutically acceptable salts, see Stahl and Wermuth, Handbook of Pharmaceutical Salts: Properties, Selection and Use (Wiley-VCH, Weinheim, Germany, 2002).

The present disclosure is directed to a method of treating cancer in a subject, the method comprising administering to the subject: (a) a KRAS inhibitor, and (b) an Ataxia Telangiectasia and Rad-3-related (ATR) inhibitor.

The present disclosure is also directed to a KRAS inhibitor for use in the treatment of cancer in a subject, wherein said treatment comprises the separate, sequential or simultaneous administration of:
a. said KRAS inhibitor, and
b. an ATR inhibitor,
   to said subject.

The present disclosure is also directed to an ATR inhibitor for use in the treatment of cancer in a subject, wherein said treatment comprises the separate, sequential or simultaneous administration of:
a. a KRAS inhibitor, and
b. said ATR inhibitor,
   to said subject.

The present disclosure is also directed to use of a KRAS inhibitor or an ATR inhibitor in the manufacture of a medicament for administration of the KRAS inhibitor and the ATR inhibitor in combination, for the treatment of cancer in a subject.

The term KRAS refers to Kirsten rat sarcoma 2 vial oncogene homolog (annotated under U.S. government's National Center for Biotechnology Information (NCBI) Genbank (http://www.ncbi.nlm.nih.gov/) Gene ID no. 3845) and refers to a small GTPase class of proteins and have been well-studied in the art. The terms “KRAS,” “the KRAS” and “KRAS protein” are used interchangeably herein. Alternative RNA splicing of the KRAS transcript leads to two known KRAS isoforms, KRAS4A and KRAS4B, that differ in the C-terminal region. A human wild-type KRAS4A isoform amino acid sequence may be as annotated under Genbank accession no: NP_203524.1 or Swissprot/Uniprot (http://www.uniprot.org/) accession no: P01116-1 (v1), the NP 203524.1 sequence reproduced in Table 1.

TABLE 1
(SEQ ID NO: 1)

MTEYKLVVVGAGGVGKSALTIQLIQNHFVDEYDPT
IEDSYRKQVVIDGETCLLDILDTAGQEEYSAMRDQ
YMRTGEGFLCVFAINNTKSFEDIHHYREQIKRVKD
SEDVPMVLVGNKCDLPSRTVDTKQAQDLARSYGIP
FIETSAKTRQRVEDAFYTLVREIRQYRLKKISKEE
KTPGCVKIKKCIIM

A human wild-type KRAS4B isoform amino acid sequence may be as annotated under Genbank accession no: NP_004976.2 or Swissprot/Uniprot accession no: P01116-2 (v1), the NP_004976.2 sequence reproduced here below in Table 2:

TABLE 2
(SEQ ID NO: 2)

MTEYKLVVVGAGGVGKSALTIQLIQNHFVDEYDPT
IEDSYRKQVVIDGETCLLDILDTAGQEEYSAMRDQ
YMRTGEGFLCVFAINNSKSFADINLYREQIKRVKD
SDDVPMVLVGNKCDLPTRTVDTKQAHELAKSYGIP
FIETSAKTRQGVEDAFYTLVREIRQYRMKKLNSSD
DGTQGCMGLPCVVM

In some embodiments, the KRAS can include other related Ras proteins, e.g., NRAS or BRAS, which are basically identical at positions 1-86, which includes the region around G12 and G13, and thus an identical molecule can be used to target G12 or G13 mutant human KRAS, NRAS and HRAS proteins. Human wild-type HRAS amino acid sequence may be as annotated under Genbank accession no: NP_005334.1 or Swissprot/Uniprot accession no: P01112 (v1), the NP_005334.1 sequence reproduced here below in Table 3:

TABLE 3
(SEQ ID NO: 3)

MTEYKLVVVGAGGVGKSALTIQLIQNHFVDEYDPTIE
DSYRKQVVIDGETCLLDILDTAGQEEYSAMRDQYMRT
GEGFLCVFAINNTKSFEDIHQYREQIKRVKDSDDVPM
VLVGNKCDLAARTVESRQAQDLARSYGIPYIETSAKT
RQGVEDAFYTLVREIRQHKLRKLNPPDESGPGCMSCK
CVLS

In human cancers, KRAS is the predominantly mutated RAS isoform (85%). Notably, the amino acid sequences of human KRAS, NRAS and HRAS are basically identical at positions 1-86, which includes the region around G12 and G13, and thus an identical molecule can be used to target G12 or G13 mutant human KRAS, NRAS and IHRAS proteins.

In some embodiments, the KRAS inhibitor can inhibit human KRAS. The qualifier “human” as used herein in connection with a KRAS protein may in a certain interpretation refer to the amino acid sequence of the KRAS protein. For example, a KRAS protein having the amino acid sequence as a KRAS protein found in humans may also be obtained by technical means, e.g., by recombinant expression, cell-free translation, or non-biological peptide synthesis. Because the present molecules are intended to therapeutically target mutant KRAS proteins in humans, in a certain other interpretation the qualifier “human” may more particularly refer to a KRAS protein as found in or present in humans, regardless of whether the KRAS protein forms a part of or has been at least partly isolated from human subjects, organs, cells, or tissues. A skilled person understands that the amino acid sequence of a given native protein such as a KRAS protein may differ between or within different individuals of the same species due to normal genetic diversity (allelic variation, polymorphism) within that species and/or due to differences in post-transcriptional or post-translational modifications. Any such variants or isoforms of the native protein are subsumed by the reference to or designation of the protein.

Various compounds that inhibit KRAS are known in the art and can include antibodies that specifically bind KRAS. In some embodiments, the KRAS inhibitor is an antibody or other therapeutic protein which can selectively bind to KRAS, i.e., an anti-KRAS antibody.

As used herein, the term “antibody” refers to a polypeptide or group of polypeptides that include at least one binding domain that is formed from the folding of polypeptide chains having three-dimensional binding spaces with internal surface shapes and charge distributions complementary to the features of an antigenic determinant of an antigen. An antibody typically has a tetrameric form, with two pairs of polypeptide chains, each pair having one “light” and one “heavy” chain, wherein the variable regions of each light/heavy chain pair form an antibody binding site. Typically, each light chain is linked to a heavy chain by one covalent disulfide bond, while the number of disulfide linkages varies between the heavy chains of different immunoglobulin isotypes. Each heavy and light chain also has regularly spaced intrachain disulfide bridges. Typically, each heavy chain has at one end a variable domain (VH) followed by a number of constant domains (CH) and each light chain has a variable domain at one end (VL) and a constant domain (CL) at its other end wherein the constant domain of the light chain is aligned with the first constant domain of the heavy chain, and the light chain variable domain is aligned with the variable domain of the heavy chain.

The terms “antibody,” “antibodies” and “immunoglobulins” as used herein encompass monoclonal antibodies (including full-length monoclonal antibodies), polyclonal antibodies, multi-specific antibodies formed from at least two different epitope binding fragments (e.g., bispecific antibodies), CDR-grafted, human antibodies, humanized antibodies, camelised antibodies, chimeric antibodies, single-chain Fvs (scFv), single-chain antibodies, single domain antibodies, Fab fragments, Fab′ fragments, F(ab′)₂ fragments, antibody fragments that exhibit a desired biological activity (e.g., the antigen binding portion), disulfide-linked Fvs (dsFv), and anti-idiotypic (anti-Id) antibodies, intrabodies, and epitope-binding fragments or derivatives of any of the above. In particular, antibodies include immunoglobulin molecules and immunologically active fragments of immunoglobulin molecules that contain at least one antigen-binding site. Immunoglobulin molecules can be of any isotype (e.g., IgG, IgE, IgM, IgD, IgA and IgY), subisotype (e.g., IgG1, IgG2, IgG3, IgG4, IgAQ1 and IgA2) or allotype (e.g., Gm, e.g., G1m(f, z, a or x), G2m(n), G3m(g, b, or c), Am, Em, and Km(1, 2 or 3)). Antibodies may be derived from any mammalian species, including, but not limited to, humans, monkeys, pigs, horses, rabbits, dogs, cats, and mice, or other animals such as birds, including, but not limited to, chickens. Antibodies may be fused to a heterologous polypeptide sequence, for example, a tag to facilitate purification.

The antibodies can be modified in the Fc region to provide desired effector functions or serum half-life. As discussed in more detail in the sections below, with the appropriate Fc regions, the naked antibody bound on the cell surface can induce cytotoxicity via antibody-dependent cellular cytotoxicity (ADCC), by recruiting complement in complement dependent cytotoxicity (CDC), or by recruiting nonspecific cytotoxic cells that express one or more effector ligands that recognize bound antibody on the Influenza A virus and subsequently cause phagocytosis of the cell in antibody dependent cell-mediated phagocytosis (ADCP), or some other mechanism. Alternatively, where it is desirable to eliminate or reduce effector function, for example, to reduce side effects or therapeutic complications, modified Fc regions may be used, for example to increase the binding affinity for FcRn and increase serum half-life. Alternatively, the Fc region can be conjugated to a moiety such as PEG or albumin to increase the serum half-life.

As used herein, the term “variant” refers to an antibody, which differs in amino acid sequence from a “parent” antibody amino acid sequence by virtue of addition, deletion and/or substitution of one or more amino acid residue(s) in the parent antibody sequence. A variant antibody may include one or more substitutions, deletions, including internal deletions, additions, including additions yielding fusion proteins, or conservative substitutions of amino acid residues of a parent antibody.

In some embodiments, the KRAS inhibitor is an antibody such as ELI-002, KRAS-EphA-2-CAR-DC, Anti-KRAS G12V mTCR PBL, anti-KRAS G12D mTCR PBL, siG12D-LODER, or KRAS G12D siRNA. In some embodiments, the anti-KRAS antibody is a monoclonal antibody.

In some embodiments, the KRAS inhibitor is a small molecule KRAS inhibitor. The term “small molecule” inhibitor, as used herein refers non-peptide based and non-nucleic acid based chemical compounds which reduce the activity of KRAS. In some embodiments, the small molecule KRAS inhibitor reduces the activity of KRAS. In some embodiments, the small molecule KRAS inhibitor increases the degradation of KRAS, thereby decreasing the activity of KRAS function. In some embodiments, the small molecule inhibitor has a molecular weight of less than 1.5 kDa, less than 1.0 kDa, or less than 700 kDa. In some embodiments, the small molecule KRAS inhibitor binds to KRAS. In some embodiments, the small molecule KRAS inhibitor has an IC50 of less than about 1 mM, less than about 100 μM, less than about 50 μM, less than about 25 μM, 10 μM, less than about 1 μM, less than about 500 nM, less than about 100 nM, less than about 50 nM, less than about 25 nM, less than about 10 nM, less than about 5 nM, or less than about 2.5 nM. In some embodiments, the small molecule KRAS inhibitor has an IC50 of less than about 2 nM or less about 1 nM. In some embodiments, the small molecule KRAS inhibitor has an IC50 of about 0.05 nM to about 50 nM, about 0.1 nM to about 10 nM, about 0.1 nM to about 25 nM, or about 0.5 nM to about 10 nM. In some embodiments, the small molecule KRAS inhibitor has an IC 50 of about 0.5 nM to about 5 nM, or about 0.5 nM to about 2 nM.

In some embodiments, the small molecule KRAS inhibitor can include small molecules known to the skilled artisan. For example, in some embodiments, the small molecule KRAS inhibitor is:
sotorasib (AMG-510, Amgen, Formula L)

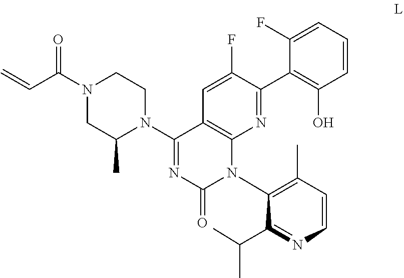

adagrasib (MRTX849, Mirati Therapeutics, Inc., Formula M)

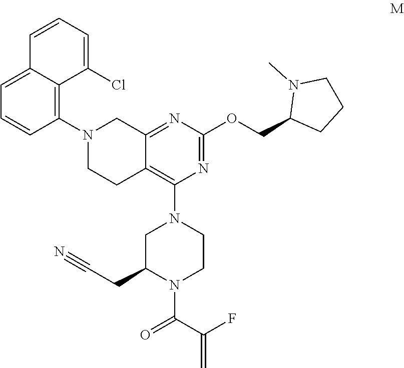 [SMILES: C=C(F)C(=O)N1CCN(c2nc(OC[C@@H]3CCCN3C)nc3c2CCN(c2cccc4cccc(Cl)c24)C3)C[C@@H]1CC#N]

Ly3537982 (Lilly, Formula N)

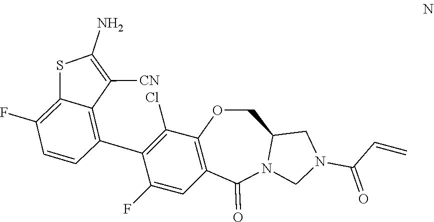 [SMILES: C=CC(=O)N1C[C@@H]2COc3c(cc(F)c(-c4ccc(F)c5sc(N)c(C#N)c45)c3Cl)C(=O)N2C1]

divarasib (GDC-6036, Genetech, Formula O)

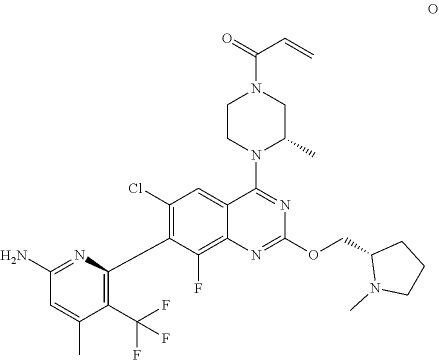

garsorasib (D-1553, InventisBio, Co., Ltd., Formula P)

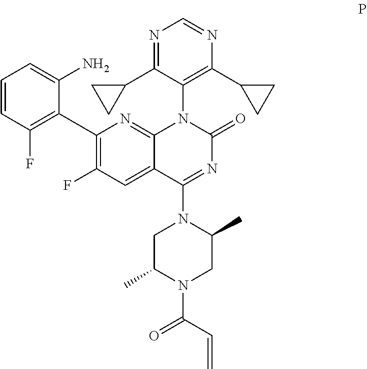 [SMILES: C=CC(=O)N1C[C@H](C)N(c2nc(=O)n(-c3c(C4CC4)ncnc3C3CC3)c3nc(-c4c(N)cccc4F)c(F)cc23)C[C@H]1C]

opnurasib (JDQ443, Novartis, Formula Q)

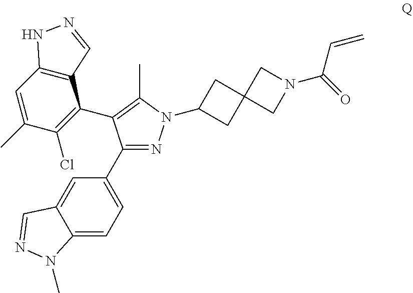

fulzerasib (IBI351, GFH925, Innovent Biologics, Formula R)

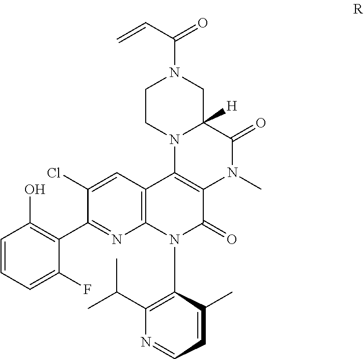

BI1823911, D35-001, glecirasib (JAB-21822, Jacobio), HBI-2438, YL-15293, GEC255, RMC-6291, MK-1084, MRTX1133, HRS-4642, RMC-9805, JAB-2200, Vrtx153, ERAS-4, ASP3082, RMC-0708, RMC-8839, RMC-6291, JAB-23400, TEB-17231, or QTX3046. In some embodiments, the KRAS inhibitor can include combinations of the small molecule KRAS inhibitors as described herein.

Small molecule KRAS inhibitors of the Formula (I) are described in WO2019215203, the content of which is hereby incorporated by reference in its entirety. In some embodiments, the small molecule KRAS inhibitor is a compound of the Formula (I).

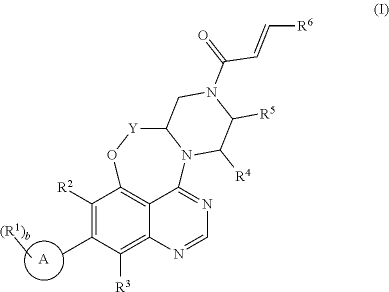

wherein:
Ring A is selected from phenyl and bicyclic heteroaryl;
R¹ in each occurrence is independently selected from C₁₋₄ alkyl, halo, hydroxy, C₁₋₄ alkoxy, C₁₋₃ fluoroalkyl, C₁₋₃ fluoroalkoxy, cyano and acetylenyl;
b is 0, 1, 2 or 3;
Y is CH₂ or CH₂CH₂;
R is cyano, halo, C₁₋₄ alkyl, C₁₋₄ alkoxy or C₁₋₃ fluoroalkyl;
R³ is F, Me, Et, MeO or C₁₋₂ fluoroalkyl;
R⁴ is H or Me;
R⁵ is H or Me;
R⁶ is H or CH₂NMe₂;
or a pharmaceutically acceptable salt thereof, provided that when Y is CH₂, R² is Cl, R³ is F, ring A is phenyl, b is 2, the R¹ groups are F and OH and are each ortho to the biaryl bond, and when both R⁴ and R⁶ are H, then R⁵ is Me.

In some embodiments of Formula (I), the small molecule KRAS inhibitor has the structure

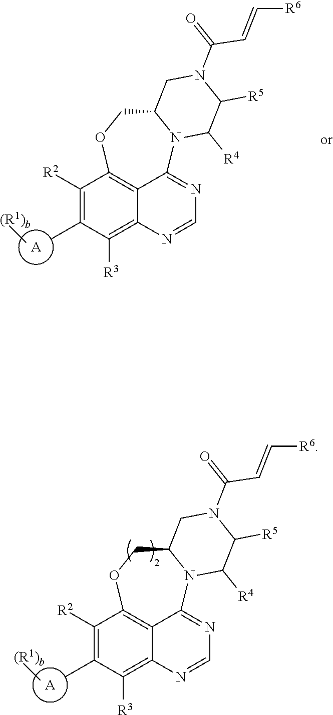

Various bicyclic aryl rings can be used for ring A of Formula (I). In some embodiments, the ring A in the compounds of Formula (I) is selected from phenyl and bicyclic heteroaryl. Bicyclic heteroaryl as used herein refers to an aromatic group comprising two fused rings and containing 1, 2, 3 or 4 N atoms, or one O atom, or one S atom, or 1 N atom and one S atom, or 1 N atom and one O atom, or 2 N atoms and one S atom, or 2 N atoms and one O atom. Bicyclic heteroaryl groups include those groups where both fused rings are aromatic, or where one fused ring is aromatic and the other fused ring is partially or fully saturated. The said partially or fully saturated fused ring may also comprise a carbonyl group. The at least one heteroatom in the bicyclic heteroaryl group may be present in an aromatic ring or a saturated ring. The bicyclic heteroaryl groups A of the compounds of Formula (I) are [6,6] or [6,5] ring systems, examples of suitable bicyclic heteroaryl groups include indolyl, benzofuranyl, benzothienyl, benzoxazolyl, benzimidazolyl, benzotriazolyl, indazolyl, azaindolyl, azaindazolyl, pyrrolo[1,2-b]pyridazinyl and pyrrolo[2,3-b]pyridinyl, quinolinyl, isoquinolinyl, quinazolinyl, cinnolinyl, phthalazinyl, quinoxalinyl and naphthyridinyl and partially saturated derivatives thereof.

In some embodiments, the ring A of Formula (I) is selected from the group consisting of:

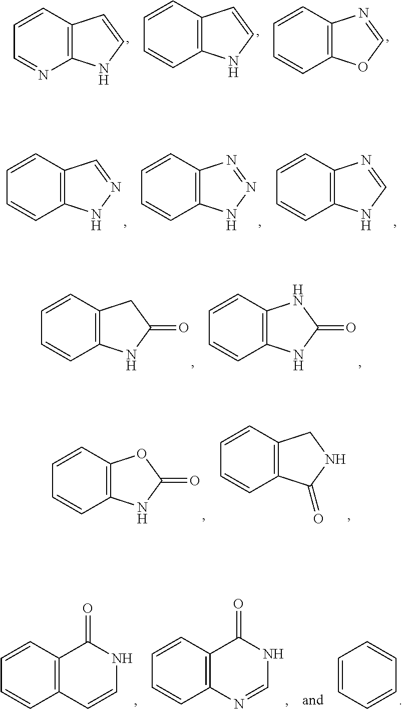

In some embodiments, the ring A as listed above is attached to the remainder of compound for Formula (I) at any point on the above listed rings. In some embodiments, the ring A as listed above is attached to the remainder of compound for Formula (I) at a carbon on the ring. In some embodiments, the ring A as listed above is attached to the remainder of compound for Formula (I) at a heteroatom on the ring. In some embodiments, the ring A as listed above is attached to the remainder of compound for Formula (I) at position 1, 2, 3, 4, 5, 6, 7, 8 or 9 of the ring.

In some embodiments, the (R¹)_{b} is attached to the ring A listed above at any point on the above listed rings. In some embodiments, the (R¹)_{b} is attached to the ring A listed above at a carbon on the ring. In some embodiments, the (R¹)_{b} is attached to the ring A listed above at a heteroatom on the ring. In some embodiments, the (R¹)_{b} is attached to the ring A listed above at position 1, 2, 3, 4, 5, 6, 7, 8 or 9 of the ring.

In some embodiments, R¹ in each occurrence is independently selected from C₁₋₄ alkyl, halo, hydroxy, C₁₋₄ alkoxy, C₁₋₃ fluoroalkyl, C₁₋₃ fluoroalkoxy, cyano and acetylenyl. For the avoidance of doubt, C₁₋₄ alkyl refers to a straight or branched alkyl group containing from 1 to 4 carbon atoms; use of numerical subscripts throughout the specification, for example for alkoxy, fluoroalkyl and fluoroalkoxy groups, is consistent with this usage.

The term halo as used herein refers to an atom selected from F, Cl, Br or I. In embodiments of the specification the halo groups in the compounds of Formula (I), and in particular for the groups R² and R¹, F and Cl are preferred halo groups.

The group R² is selected from cyano, halo, C₁₋₄ alkyl, C₁₋₄ alkoxy or C₁₋₃ fluoroalkyl. Examples of preferred R² groups include Cl, methyl and cyano, for example Cl. The group R³ is selected from F, Me, Et, MeO or C₁₋₂ fluoroalkyl, for example F, Me or MeO.

For the avoidance of doubt, where multiple substituents are independently selected from a given group, the selected substituents may comprise the same substituents or different substituents from within the given group. By way of example only, where ring A of Formula (I) is phenyl substituted with (R¹)_{b}, and where b is 2, the two R¹ groups could be the same, for instance both fluoro, or could be different, for instance one fluoro and one hydroxy.

For the further avoidance of doubt, the use of “ [symbol] ” in formulas of this specification denotes the point of attachment between different groups.

As noted above, in some embodiments, the small molecule KRAS inhibitor is a compound of the Formula (I):

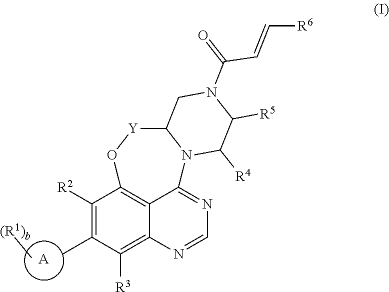

wherein:
Ring A is selected from phenyl and bicyclic heteroaryl;
R¹ in each occurrence is independently selected from C₁₋₄ alkyl, halo, hydroxy, C₁₋₄ alkoxy, C₁₋₃ fluoroalkyl, C₁₋₃ fluoroalkoxy, cyano and acetylenyl;
b is 0, 1, 2 or 3;
Y is CH₂ or CH₂CH₂;
R² is cyano, halo, C₁₋₄ alkyl, C₁₋₄ alkoxy or C₁₋₃ fluoroalkyl;
R³ is F, Me, Et, MeO or C₁₋₂ fluoroalkyl;
R⁴ is H or Me;
R⁵ is H or Me;
R⁶ is H or CH₂NMe₂;
or a pharmaceutically acceptable salt thereof, provided that when Y is CH₂, R² is Cl, R³ is F, A is phenyl, b is 2, the R¹ groups are F and OH and are each ortho to the biaryl bond, and when both R⁴ and R⁶ are H, then R⁵ is Me.

In one embodiment, the small molecule KRAS inhibitor is a compound of Formula (I) as defined above.

In one embodiment, the small molecule KRAS inhibitor is a pharmaceutically acceptable salt of a compound of Formula (I).

In embodiments, the compound of Formula (I) is a compound of Formula (Ia) wherein Y is CH₂.

In embodiments, the compound of Formula (I) is a compound of Formula (Ib) wherein Y is CH₂CH₂.

In embodiments, the compound of Formula (I), (Ia) or (Ib) is a compound of Formula (Ic) in which R² is selected from Cl, Me, or CN (cyano). In embodiments, the compound of Formula (Ic) is a compound of Formula (Id) in which R² is Cl.

In embodiments, the compound of Formula (I), (Ia), (Ib), (Ic) or (Id) is a compound of Formula (Ie) in which R³ is selected from F, Me or MeO. In embodiments, the compound of Formula (Ie) is a compound of Formula (If) in which R³ is F.

In embodiments, the compound of Formula (I), (Ia), (Ib), (Ic), (Id), (le) or (If) is a compound of Formula (Ig) in which R⁴ is H.

In embodiments, the compound of Formula (I), (Ia), (Ib), (Ic), (Id), (le), (If) or (Ig) is a compound of Formula (Ih) in which R⁵ is H. In embodiments, the compound of Formula (I), (Ia), (Ib), (Ic), (Id), (le), (If) or (Ig) is a compound of Formula (Ii) in which R⁵ is Me.

In embodiments, the compound of Formula (I), (Ia), (Ib), (Ic), (Id), (le), (If), (Ig), (Ih) or (Ii) is a compound of Formula (Ij) in which R⁶ is H.

In embodiments, the compound of Formula (I), (Ia), (Ib), (Ic), (Id), (le), (If), (Ig), (Ih), (Ii) or (Ij) is a compound of Formula (Ik) in which ring A is phenyl.

In embodiments, the compound of Formula (Ik) is a compound of Formula (Il) in which the integer b is 2 or 3 and at least one R¹ group is OH. In embodiments, the compound of Formula (Ik) or (Il) is a compound of Formula (Im) in which at least two R¹ groups are ortho to the biaryl bond.

In embodiments, the compound of Formula (Ik) is a compound of Formula (In) in which A(R¹)_{b} is

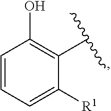

optionally wherein R¹ is selected from Me, F, Cl and CN (cyano). In embodiments, the R¹ in the compound of Formula (In) is selected from Me, Cl and CN.

In embodiments, the compound of Formula (Ik) is a compound of Formula (Io) in which A(R¹)_{b} is

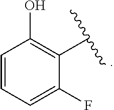 [SMILES: *c1c(O)cccc1F]

In embodiments the compound of Formula (I), (Ia), (Ib), (Ic), (Id), (Ie), (If), (Ig), (Ih), (Ii) or (Ij) is a compound of Formula (Ip) in which ring A is bicyclic heteroaryl.

In embodiments, the compound of Formula (Ip) is a compound of Formula (Iq) in which the bicyclic heteroaryl group of ring A is selected from the group consisting of:

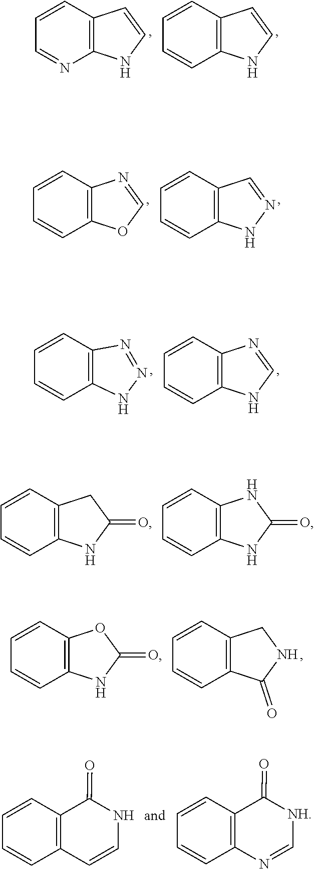

In some embodiments, the bicyclic heteroaryl group of ring A as listed above is attached to the remainder of compound of Formula (Ip) at any point on the above listed rings. In some embodiments, the bicyclic heteroaryl group of ring A as listed above is attached to the remainder of compound of Formula (Ip) at a carbon on the ring. In some embodiments, the bicyclic heteroaryl group of ring A as listed above is attached to the remainder of compound of Formula (Ip) at a heteroatom on the ring. In some embodiments, the bicyclic heteroaryl group of ring A as listed above is attached to the remainder of compound of Formula (Ip) at position 1, 2, 3, 4, 5, 6, 7, 8 or 9 of the ring.

In some embodiments, the (R¹)_{b} is attached to the ring A listed above at any point on the above listed rings for Formula (Ip). In some embodiments, the (R¹)_{b} is attached to the ring A listed above at a carbon on the ring for Formula (Ip). In some embodiments, the (R¹)_{b} is attached to the ring A listed above at a heteroatom on the ring for Formula (Ip). In some embodiments, the (R¹)_{b} is attached to the ring A listed above at position 1, 2, 3, 4, 5, 6, 7, 8 or 9 of the ring for Formula (Ip).

In embodiments, the compound of Formula (Ip) is a compound of Formula (Ir) in which the bicyclic heteroaryl group is selected from the group consisting of:

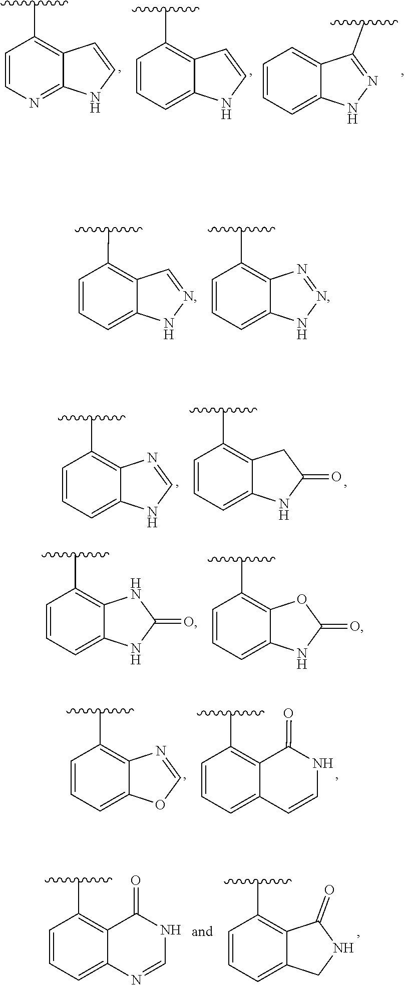

In embodiments, the compound of Formula (Ip) is a compound of Formula (Is) in which the bicyclic heteroaryl group is selected from the group consisting of:

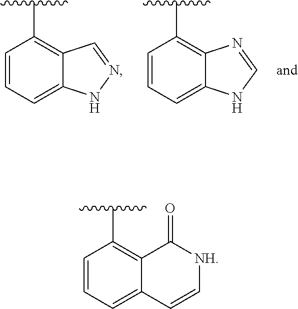

In embodiments, the compound of Formula (Ip) is a compound of Formula (It) in which the bicyclic heteroaryl group is selected from the group consisting of:

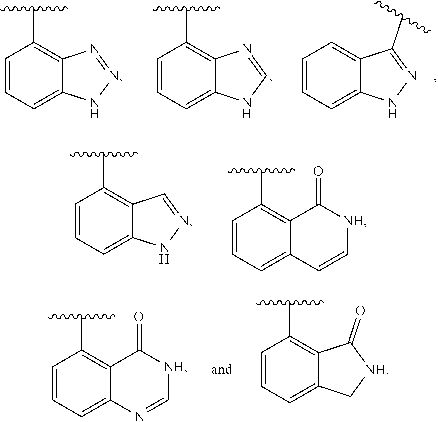

In embodiments, the compounds of Formula (I), i.e. any of compounds of Formula (I), (Ia), (Ib) . . . to (It), is a compound of Formula (Iu) or (Iv) in which the stereochemistry is as shown below:

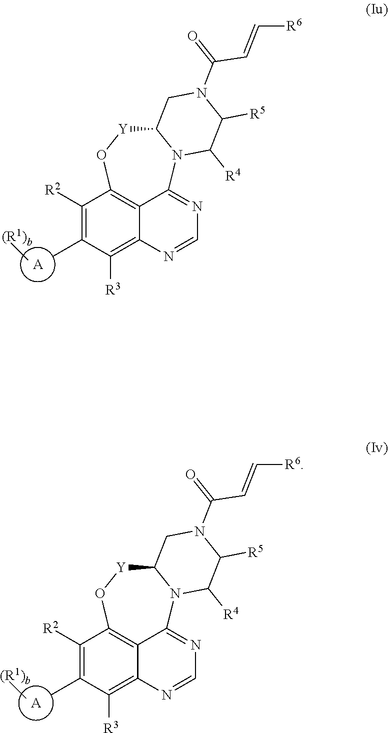

In embodiments, the compound of Formula (Iu) is a compound of Formula (Iui) in which Y=CH₂. In embodiments, the compound of Formula (Iv) is a compound of Formula (Ivi) in which Y=CH₂CH₂.

In embodiments, the compounds of Formula (I), i.e., any of compounds of Formula (I), (Ia), (Ib) . . . to (Ivi), is a compound of Formula (Iw) in which R⁴ is H and R⁵ is Me.

In embodiments, the compound of Formula (I) is selected from each enantiomeric and atropisomeric form of:
7-[(8aS)-10-Acryloyl-6-chloro-4-fluoro-8,8a,9,10,11,12-hexahydropyrazino[2′,1′:3,4][1,4]oxazepino [5,6,7-de]quinazolin-5-yl]-6-methyl-2,3-dihydro-1H-isoindol-1-one;
1-[(8aS,11S)-6-Chloro-4-fluoro-5-(2-fluoro-6-hydroxyphenyl)-11-methyl-8a,9,11,12-tetrahydropyrazino[2′,1′:3,4][1,4]oxazepino[5,6,7-de]quinazolin-10(8H)-yl]prop-2-en-1-one;
1-[(8aS,11R)-6-Chloro-4-fluoro-5-(2-fluoro-6-hydroxyphenyl)-11-methyl-8a,9,11,12-tetrahydropyrazino[2′,1′:3,4][1,4]oxazepino[5,6,7-de]quinazolin-10(8H)-yl]prop-2-en-1-one;
5-[(8aS)-10-Acryloyl-6-chloro-4-fluoro-8,8a,9,10,11,12-hexahydropyrazino[2′,1′:3,4][1,4]oxazepino[5,6,7-de]quinazolin-5-yl]-6-methylquinazolin-4(3H)-one;
1-[(8aS)-6-Chloro-4-fluoro-5-(5-methyl-1H-benzimidazol-4-yl)-8a,9,11,12-tetrahydropyrazino [2′,1′:3,4][1,4]oxazepino[5,6,7-de]quinazolin-10(8H)-yl]prop-2-en-1-one;
8-[(8aS)-6-Chloro-4-fluoro-10-(prop-2-enoyl)-8,8a,9,10,11,12-hexahydropyrazino[2′,1′:3,4][1,4]oxazepino[5,6,7-de]quinazolin-5-yl]isoquinolin-1(2H)-one;
1-[(8aS)-6-Chloro-4-fluoro-5-(1H-indazol-3-yl)-8a,9,11,12-tetrahydropyrazino[2′,1′:3,4][1,4]oxazepino[5,6,7-de]quinazolin-10(8H)-yl]prop-2-en-1-one;
1-[(8aS)-6-Chloro-4-fluoro-5-(2-hydroxy-6-methylphenyl)-8a,9,11,12-tetrahydropyrazino [2′,1′:3,4][1,4]oxazepino[5,6,7-de]quinazolin-10(8H)-yl]prop-2-en-1-one;
(2E)-1-[(8aS)-6-Chloro-4-fluoro-5-(2-fluoro-6-hydroxyphenyl)-8a,9,11,12-tetrahydropyrazino [2′,1′:3,4][1,4]oxazepino[5,6,7-de]quinazolin-10(8H)-yl]-4-(dimethylamino)but-2-en-1-one;
8-[(8aS)-6-Chloro-4-fluoro-10-(prop-2-enoyl)-8,8a,9,10,11,12-hexahydropyrazino[2′,1′:3,4][1,4]oxazepino[5,6,7-de]quinazolin-5-yl]-7-methylisoquinolin-1(2H)-one;
1-[(8aS)-6-Chloro-4-fluoro-5-(5-methyl-1H-benzotriazol-4-yl)-8a,9,11,12-tetrahydropyrazino [2′,1′:3,4][1,4]oxazepino[5,6,7-de]quinazolin-10(8H)-yl]prop-2-en-1-one;
1-((8aS)-6-chloro-4-fluoro-5-(5-fluoro-1H-benzo[d]imidazol-4-yl)-8a,9,11,12-tetrahydropyrazino [2′,1′:3,4][1,4]oxazepino[5,6,7-de]quinazolin-10(8H)-yl)prop-2-en-1-one;
1-[(8aS)-6-Chloro-4-fluoro-5-(5-fluoro-1H-indazol-4-yl)-8a,9,11,12-tetrahydropyrazino[2′,1′:3,4][1,4]oxazepino[5,6,7-de]quinazolin-10(8H)-yl]prop-2-en-1-one;
1-((8aS)-6-Chloro-4-fluoro-5-(5-fluoro-1-methyl-1H-benzo[d]imidazol-4-yl)-8a,9,11,12-tetrahydropyrazino[2′,1′:3,4][1,4]oxazepino[5,6,7-de]quinazolin-10(8H)-yl)prop-2-en-1-one;
1-[(8aS)-6-Chloro-4-fluoro-5-(5-fluoro-1H-benzotriazol-4-yl)-8a,9,11,12-tetrahydropyrazino[2′,1′:3,4][1,4]oxazepino[5,6,7-de]quinazolin-10(8H)-yl]prop-2-en-1-one;
8-[(8aS)-6-Chloro-4-fluoro-10-(prop-2-enoyl)-8,8a,9,10,11,12-hexahydropyrazino[2′,1′:3,4][1,4]oxazepino[5,6,7-de]quinazolin-5-yl]-7-fluoroisoquinolin-1(2H)-one;
(2E)-1-[(8aS)-6-chloro-4-fluoro-5-(5-methyl-1H-indazol-4-yl)-8a,9,11,12-tetrahydropyrazino[2′,1′:3,4][1,4]oxazepino[5,6,7-de]quinazolin-10(8H)-yl]-4-(dimethylamino)but-2-en-1-one;
1-[(6aR,9S)-3-Chloro-1-fluoro-2-(2-fluoro-6-hydroxyphenyl)-9-methyl-5,6,6a,7,9,10-hexahydro-8H-pyrazino[1′,2′:5,6][1,5]oxazocino[4,3,2-de]quinazolin-8-yl]prop-2-en-1-one;
1-[(6aR,9S)-3-Chloro-1-fluoro-2-(2-fluoro-6-hydroxyphenyl)-9-methyl-5,6,6a,7,9,10-hexahydro-8H-pyrazino[1′,2′:5,6][1,5]oxazocino[4,3,2-de]quinazolin-8-yl]prop-2-en-1-one;
8-[3-Chloro-1-fluoro-8-(prop-2-enoyl)-6,6a,7,8,9,10-hexahydro-5H-pyrazino[1′,2′:5,6][1,5]oxazocino [4,3,2-de]quinazolin-2-yl]-7-methylisoquinolin-1(2H)-one;
1-[(6aS,9R)-3-Chloro-1-fluoro-2-(2-fluoro-6-hydroxyphenyl)-9-methyl-5,6,6a,7,9,10-hexahydro-8H-pyrazino[1′,2′:5,6][1,5]oxazocino[4,3,2-de]quinazolin-8-yl]prop-2-en-1-one;
1-[(6aR,9R)-3-Chloro-1-fluoro-2-(2-fluoro-6-hydroxyphenyl)-9-methyl-5,6,6a,7,9,10-hexahydro-8H-pyrazino[1′,2′:5,6][1,5]oxazocino[4,3,2-de]quinazolin-8-yl]prop-2-en-1-one;
1-[(6aS,9S)-3-Chloro-1-fluoro-2-(2-fluoro-6-hydroxyphenyl)-9-methyl-5,6,6a,7,9,10-hexahydro-8H-pyrazino[1′,2′:5,6][1,5]oxazocino[4,3,2-de]quinazolin-8-yl]prop-2-en-1-one;
1-[(8aS)-4-Chloro-6-fluoro-5-(2-fluoro-6-hydroxyphenyl)-8a,9,11,12-tetrahydropyrazino[2′,1′:3,4][1,4]oxazepino[5,6,7-de]quinazolin-10(8H)-yl]prop-2-en-1-one;
1-[(8aS,11R)-6-Chloro-4-fluoro-11-methyl-5-(5-methyl-1H-benzimidazol-4-yl)-8a,9,11,12-tetrahydropyrazino[2′,1′:3,4][1,4]oxazepino[5,6,7-de]quinazolin-10(8H)-yl]prop-2-en-1-one;
8-[(8aS,11R)-6-Chloro-4-fluoro-11-methyl-10-(prop-2-enoyl)-8,8a,9,10,11,12-hexahydropyrazino [2′,1′:3,4][1,4]oxazepino[5,6,7-de]quinazolin-5-yl]-7-methylisoquinolin-1(2H)-one;
(2E)-1-[(8aS,11R)-6-Chloro-4-fluoro-11-methyl-5-(5-methyl-1H-benzimidazol-4-yl)-8a,9,11,12-tetrahydropyrazino[2′,1′:3,4][1,4]oxazepino[5,6,7-de]quinazolin-10(8H)-yl]-4-(dimethylamino)but-2-en-1-one; and
(2E)-1-[(8aS,11R)-6-Chloro-4-fluoro-11-methyl-5-(5-methyl-1H-indazol-4-yl)-8a,9,11,12-tetrahydropyrazino[2′,1′:3,4][1,4]oxazepino[5,6,7-de]quinazolin-10(8H)-yl]-4-(dimethylamino)but-2-en-1-one;
or a pharmaceutically acceptable salt thereof.

In some embodiments, the small molecule KRAS inhibitor is a compound of Formula (II):

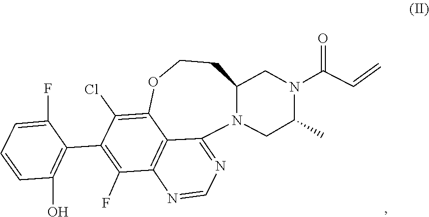 [SMILES: C=CC(=O)N1C[C@@H]2CCOc3c(Cl)c(-c4c(O)cccc4F)c(F)c4ncnc(c34)N2C[C@H]1C]

which is 1-[(6aS,9R)-3-chloro-1-fluoro-2-(2-fluoro-6-hydroxyphenyl)-9-methyl-5,6,6a,7,9,10-hexahydro-8H-pyrazino[1′,2′:5,6][1,5]oxazocino[4,3,2-de]quinazolin-8-yl]prop-2-en-1-one, or a pharmaceutically acceptable salt thereof.

Pharmaceutical compositions which comprise a compound of the Formula (I) or a pharmaceutically acceptable salt thereof, in association with a pharmaceutically acceptable excipient, optionally further comprise one or more of the other stereoisomeric forms of the compound of Formula (I) or a pharmaceutically acceptable salt thereof, wherein the compound of Formula (I) or pharmaceutically acceptable salt thereof is present within the composition with a diastereomeric excess (% d.e.) of ≥90%.

Pharmaceutical compositions which comprise a compound of the Formula (I) or a pharmaceutically acceptable salt thereof, in association with a pharmaceutically acceptable excipient, optionally further comprise one or more of the other stereoisomeric forms of the compound of Formula (I) or a pharmaceutically acceptable salt thereof, wherein the compound of Formula (I) or pharmaceutically acceptable salt thereof is present within the composition with an enantiomeric excess (% ee) of ≥90% and a diastereomeric excess (% de) of ≥90%.

The compounds of Formula (I) and pharmaceutically acceptable salts thereof may be prepared, used or supplied in amorphous form, crystalline form, or semi-crystalline form and any given compound of Formula (I) or pharmaceutically acceptable salt thereof may be capable of being formed into more than one crystalline/polymorphic form, including hydrated (e.g. hemi-hydrate, a mono-hydrate, a di-hydrate, a tri-hydrate or other stoichiometry of hydrate) and/or solvated forms. It is to be understood that the present specification encompasses any and all such solid forms of the compound of Formula (I) and pharmaceutically acceptable salts thereof.

Small molecule KRAS inhibitors of Formula (A) are described in WO2020178282, the content of which is hereby incorporated by reference in its entirety. In one embodiment, the small molecule KRAS inhibitor is a compound of Formula (A):

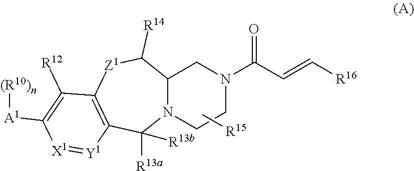

wherein:
A¹ is phenyl or a bicyclic heteroaryl group;
X¹ and Y¹ are connected by a double bond and i) X¹ is CR¹⁷ and Y¹ is CR¹⁸, ii) X¹ is N and Y¹ is CR¹⁸, or iii) X¹ is CR¹⁷ and Y¹ is N; or
X¹ and Y¹ together are C(O)NR¹⁹; or
X¹ and Y¹ are adjacent ring atoms of an optionally substituted 5- or 6-membered N-heterocycle fused to the aromatic ring substituted with Z¹, and X¹ and Y¹ are both C or are C and N;
Z¹ is O, NH, or NMe;
R¹⁰ is independently selected from F, Cl, Br, OH, CH₂OH, OMe, CH₂OMe, C₁-C₃ alkyl and C₁-C₃ fluoroalkyl;
n is 0, 1, 2 or 3;
R¹² is H, F, Cl, CCH, CCMe, CN, Br, C₁-C₃ alkyl, C₁-C₃ fluoroalkyl, OMe or OEt;
R^{13a} and R^{13b} together are ═O or R^{13a} and R^{13b} are H;
R¹⁴ is H or Me;
R¹⁵ is H or Me;
R¹⁶ is H or CH₂NMe₂;
R¹⁷ and R¹⁸ are independently selected from H, F, Cl, CCH, CC(C₁-C₃ alkyl), CCCH₂NMe₂, CCCH₂O(C₁-C₃ alkyl), CN, Me, C₁-C₆ alkyl, OH, OMe, O(C₁-C₃ alkyl), O(C₁-C₃ deuteroalkyl), O(C₁-C₃ fluoroalkyl), O(C₃-C₆ cycloalkyl), C₁-C₃ fluoroalkyl, OCH₂CH₂NMe₂, OCH₂CH₂OMe, CH₂OMe, OCH₂CH₂N(CH₂CH₂)₂CH, OCH₂CH₂N(CH₂CH₂)₂O, OCH₂CH₂(2-pyridyl) or an optionally substituted 3-, 4-, 5- or 6-membered carbocycle or heterocycle; or
R¹⁷ and R¹⁸ combine to form an optionally substituted 5- or 6-membered carbocycle or heterocycle;
R¹⁹ is selected from H, Me, Et, C₃H₇ and C₁-C₃ fluoroalkyl;
or a pharmaceutically acceptable salt thereof.

In one embodiment, the compound of Formula (A) is a compound of Formula (III):

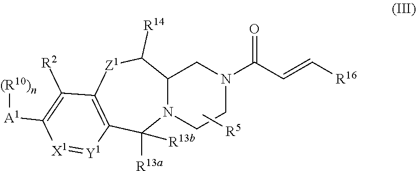

wherein:
A¹ is phenyl or a bicyclic heteroaryl group;
X¹ and Y¹ are connected by a double bond and i) X¹ is CR¹⁷ and Y¹ is CR¹⁸, ii) X¹ is N and Y¹ is CR¹⁸ or iii) X¹ is CR¹⁷ and Y¹ is N; or
X¹ and Y¹ together are C(O)NR¹⁹; or
X¹ and Y¹ are adjacent ring atoms of an optionally substituted 5- or 6-membered N-heterocycle fused to the aromatic ring substituted with Z¹, and X¹ and Y¹ are both C or are C and N;
Z¹ is O, NH, or NMe;
R¹⁰ is independently selected from F, Cl, Br, OH, CH₂OH, OMe, CH₂OMe, C₁-C₃ alkyl, and C₁-C₃fluoroalkyl;
n is 0, 1, 2 or 3;
R¹² is H, F, Cl, CCH, CCMe, CN, Br, C₁-C₃ alkyl, C₁-C₃ fluoroalkyl, OMe or OEt;
R^{13a} and R^{13b} together are ═O or R^{13a} and R^{13b} are H;
R¹⁴ is H or Me;
R¹⁵ is H or Me;
R¹⁶ is H or CH₂NMe₂;
R¹⁷ and R¹⁸ are selected from H, F, Cl, CCH, CN, Me, OH, OMe, O(C₁-C₃ alkyl), C₁-C₃fluoroalkyl or an optionally substituted 5- or 6-membered carbocycle or heterocycle; or
R¹⁷ and R¹⁸ combine to form an optionally substituted 5- or 6-membered carbocycle or heterocycle;
R¹⁹ is selected from H, Me, Et, C₃H₇ and C₁-C₃ fluoroalkyl;
or a pharmaceutically acceptable salt thereof.

The compounds of Formula (A), for example compounds of Formula (III), feature a [6,7,6]-tricyclic core in which an aromatic ring containing the groups X¹ and Y¹ is linked to a piperazine by a 1,4-diazepane (Z¹=N) or 1,4-oxazepane (Z¹=O) motif. In addition, a group A¹ that is selected from phenyl and bicyclic heteroaryl, is linked through a biaryl bond to the aromatic ring containing the groups X¹ and Y¹. Subject to the nature of the groups R¹⁰, R¹² and X¹ rotation around the biaryl bond is restricted and the compounds of Formula (III), as a result, can exist in stable atropisomeric forms. An acrylamide motif is attached to tricyclic core via the non-bridgehead piperazine nitrogen.

As used herein the term “alkyl” refers to both straight and branched chain saturated hydrocarbon radicals having the specified number of carbon atoms. As used herein the term deuteroalkyl refers to an alkyl groups in which one or more, optionally all, hydrogens are replaced with deuterium atoms. The term cycloalkyl refers to a saturated carbocycle.

The term acetylenyl refers to an ethynyl radical i.e. a —CCH group.

In this specification the prefix Cₓ-C_{y}, as used in terms such as Cₓ-C_{y} alkyl and the like where x and y are integers, indicates the numerical range of carbon atoms that are present in the group. For example, C₁-C₄ alkyl includes methyl, ethyl, n-propyl, i-propyl, n-butyl, s-butyl, i-butyl and t-butyl, while examples of C₁-C₃ alkyl groups include methyl, ethyl, n-propyl, and i-propyl. C₁-C₄ alkoxy groups include methoxy, ethoxy, n-propoxy, i-propoxy, n-butoxy, sec-butoxy and t-butoxy. Examples of C₁-C₃ alkoxy groups include methoxy, ethoxy, n-propoxy and i-propoxy. Examples of C₁-C₃ fluoroalkyl groups include fluoromethyl, difluoromethyl, trifluoromethyl, 1,1-difluoroethyl and 2,2,2-trifluoroethyl. Examples of C₁-C₃ fluoroalkoxy groups include fluoromethoxy, difluoromethoxy, trifluoromethoxy and 2,2,2-trifluoroethoxy. A —O(C₁-C₃ deuteroalkyl) group is a partially or fully deuterated O-methyl, O-ethyl or O-n-propyl or O-1-propyl group. A C₃-C₆cycloalkyl group refers to a cyclopropyl, cyclobutyl, cyclopentyl or cyclohexyl group. A 2-pyridyl group is a pyridine ring attached by a bond meta to the pyridine N-atom, i.e., a group

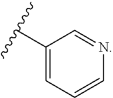 [SMILES: *c1cccnc1]

Unless specifically stated, the bonding of an atom or group may be any suitable atom of that group; for example, propyl includes prop-1-yl and prop-2-yl.

Unless otherwise stated, halo is selected from Cl, F, Br and I, generally from Cl, F or Br, or Cl and F.

As noted above, A¹ may be a phenyl group or a bicyclic heteroaryl group. A bicyclic heteroaryl group in this context is an aromatic group comprising two fused rings and containing 1, 2, 3 or 4 N atoms, or one O atom, or one S atom, or 1 N atom and one S atom, or 1 N atom and one O atom, or 2 N atoms and one S atom, or 2 N atoms and one O atom. Bicyclic heteroaryl groups include those groups where both fused rings are aromatic, or where one fused ring is aromatic and the other fused ring is partially or fully saturated. The said partially or fully saturated fused ring may also comprise a carbonyl group. Examples of suitable bicyclic heteroaryl groups include indolyl, benzofuranyl, benzothienyl, benzoxazolyl, benzimidazolyl, benzotriazolyl, indazolyl, azaindolyl, azaindazolyl, pyrrolo[1,2-b]pyridazinyl, pyrrolo[2,3-b]pyridinyl, quinolinyl, isoquinolinyl, quinazolinyl, cinnolinyl, phthalazinyl, quinoxalinyl and naphthyridinyl.

As noted above, X¹ and Y¹ in the compounds of Formula (III) can be adjacent ring atoms of a 5- or 6-membered N-heterocycle fused to the aromatic ring substituted with Z¹ and are both C or are C and N. The term 5- or 6-membered N-heterocycle refers to a saturated or unsaturated, for example aromatic, 5- or 6-membered rings containing at least one nitrogen atom and up to two further heteroatoms selected from O, N and S. The 5-membered N-heterocycle fused to the aromatic ring substituted with Z¹ can be selected from pyrrole, imidazole, pyrazole, 1,2,3-triazole, 1,2,4-triazole, oxazole, isoxazole, oxadiazole, thiazole and isothiazole and partially saturated equivalents thereof. The 6-membered N-heterocycle fused to the aromatic ring substituted with Z¹ can be selected from pyridine, pyridazine, pyrimidine and pyrazine. The 5- or 6-membered N-heterocycle may be optionally substituted with one or two substituents selected from C₁-C₃ alkyl, C₁-C₃ fluoroalkyl, C₁-C₃ alkoxy, Cl, F, CN, OH, OMe, OEt, NH₂, NHMe, NMe₂ and C₁-C₃ alkyl optionally substituted with OH, OMe, NH₂, NHMe or NMe₂.

As noted above, R¹⁷ and R¹⁸ in the compounds of Formula (III) can be an optionally substituted 5- or 6-membered carbocycle or heterocycle. The term optionally substituted 5- or 6-membered carbocycle or heterocycle refers to a saturated or unsaturated, for example aromatic, ring containing up to three heteroatoms selected from O, N and S. The 5-membered heterocycle may be selected from pyrrole, imidazole, pyrazole, 1,2,3-triazole, 1,2,4-triazole, oxazole, isoxazole, 1,2,3-oxadiazole, thiazole, isothiazole and partially or fully saturated equivalents thereof. The 6-membered heterocycle may be selected from pyridine, pyridazine, pyrimidine and pyrazine. The 5- or 6-membered carbocycle or heterocycle may be optionally substituted with one or two substituents selected from C₁-C₃ alkyl, C₁-C₃ fluoroalkyl, C₁-C₃ alkoxy, Cl, F, CN, OH, OMe, OEt, NH₂, NHMe, NMe₂ and C₁-C₃ alkyl optionally substituted with OH, OMe, NH₂, NHMe or NMe₂.

As noted above, in the instance where R¹⁷ and R¹⁸ are both present in the compounds of Formula (A), for example in compounds of Formula (III), they can combine to form an optionally substituted 5- or 6-membered carbocycle or heterocycle fused to the aromatic ring substituted with Z¹. The optionally substituted 5- or 6-membered carbocycle or heterocycle may be saturated or unsaturated. The 5- or 6-membered carbocycle or heterocycle may be optionally substituted with one or two substituents selected from C₁-C₃ alkyl, C₁-C₃ fluoroalkyl, C₁-C₃ alkoxy, Cl, F, CN, OH, OMe, OEt, NH₂, NHMe, NMe₂ and C₁-C₃ alkyl optionally substituted with OH, OMe, NH₂, NHMe or NMe₂. In the case where R¹⁷ and R¹⁸ combine to form a 5-membered ring, together they may represent a C₃, C₂O, COC, C₂N, CNC, CNO, NCO, CNS or NCS chain. In the case where R¹⁷ and R¹⁸ combine to form a 6-membered ring, together they may represent a C₄, C₃O, COC₂, OC₂O, C₃N, C₂NC, NCNC, CNNC or NCCN chain. The R¹⁷ and R¹⁸ chains of atoms are covalently bound and substituted with hydrogen or the optional substituents to satisfy their normal valency.

For the avoidance of doubt, where multiple substituents are independently selected from a given group, the selected substituents may comprise the same substituents or different substituents from within the given group. By way of example only, where A¹ is phenyl substituted with (R¹⁰)ₙ, and where n is 2, the two R¹⁰ substituents could be the same, for instance both fluoro, or could be different, for instance one fluoro and one hydroxy.

For the further avoidance of doubt, the use of “w” in formulas of this specification denotes the point of attachment between different groups.

Where any embodiment within this specification includes a group which is said to be “optionally substituted”, then a further embodiment will include that embodiment wherein the said group is unsubstituted.

In one embodiment, the small molecule KRAS inhibitor is a compound of Formula (A):

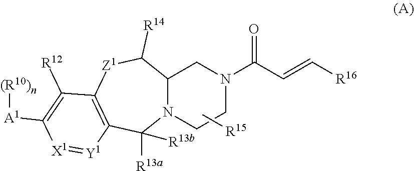

wherein:
A¹ is phenyl or a bicyclic heteroaryl group;
X¹ and Y¹ are connected by a double bond and i) X¹ is CR¹⁷ and Y¹ is CR¹⁸, ii) X¹ is N and Y¹ is CR¹⁸, or iii) X¹ is CR¹⁷ and Y¹ is N; or
X¹ and Y¹ together are C(O)NR¹⁹; or
X¹ and Y¹ are adjacent ring atoms of an optionally substituted 5- or 6-membered N-heterocycle fused to the aromatic ring substituted with Z¹, and X¹ and Y¹ are both C or are C and N;
Z¹ is O, NH, or NMe;
R¹⁰ is independently selected from F, Cl, Br, OH, CH₂OH, OMe, CH₂OMe, C₁-C₃ alkyl and C₁-C₃ fluoroalkyl;
n is 0, 1, 2 or 3;
R¹² is H, F, Cl, CCH, CCMe, CN, Br, C₁-C₃alkyl, C₁-C₃ fluoroalkyl, OMe or OEt;
R^{13a} and R^{13b} together are ═O or R^{13a} and R^{13b} are H;
R¹⁴ is H or Me;
R¹⁵ is H or Me;
R¹⁶ is H or CH₂NMe₂;
R¹⁷ and R¹⁸ are independently selected from H, F, Cl, CCH, CC(C₁-C₃ alkyl), CCCH₂Nme₂, CCCH₂O(C₁-C₃ alkyl), CN, Me, C₁-C₆ alkyl, OH, OMe, O(C₁-C₃ alkyl), O(C₁-C₃deuteroalkyl), O(C₁-C₃ fluoroalkyl), O(C₃-C₆ cycloalkyl), C₁-C₃ fluoroalkyl, OCH₂CH₂Nme₂, OCH₂CH₂Ome, CH₂OMe, OCH₂CH₂N(CH₂CH₂)₂CH, OCH₂CH₂N(CH₂CH₂)₂O, OCH₂CH₂(2-pyridyl) or an optionally substituted 3-, 4-, 5- or 6-membered carbocycle or heterocycle; or
R¹⁷ and R¹⁸ combine to form an optionally substituted 5- or 6-membered carbocycle or heterocycle;
R¹⁹ is selected from H, Me, Et, C₃H₇ and C₁-C₃ fluoroalkyl;
or a pharmaceutically acceptable salt thereof.

In embodiments, the compound of Formula (A) is a compound of Formula (Aa) in which R¹⁶ is H.

In embodiments, the compound of Formula (A) or (Aa), is a compound of Formula (Ab) in which R¹⁵ is H.

In embodiments, the compound of Formula (A), (Aa) or (Ab) is a compound of Formula (Ac) in which R¹⁴ is H.

In embodiments, the compound of Formula (A), (Aa), (Ab) or (Ac) is a compound of Formula (Ad) in which A is phenyl.

In embodiments, the compound of Formula (A) is a compound of Formula (Ae)

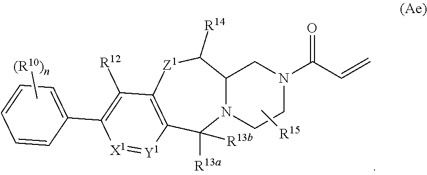

In embodiments, the compound of Formula (Ae) is a compound of Formula (Af) in which R¹⁵ is H.

In embodiments, the compound of Formula (Ae) or (Af) is a compound of Formula (Ag) in which R^{13a} and R^{13b} are H.

In embodiments, the compound of Formula (Ae) or (Af) is a compound of Formula (Ah) in which R^{13a} and R^{13b} together are ═O.

In embodiments, the compound of Formula (Ae), (Af), (Ag) or (Ah) is a compound of Formula (Ai) in which Z¹ is O.

In embodiments, the compound of Formula (Ae), (Af), (Ag), (Ah) or (Ai) is a compound of Formula (Aj) in which R¹² is selected from F or Cl.

In embodiments, the compound of Formula (Ae), (Af), (Ag), (Ah), (Ai) or (Aj) is a compound of Formula (Ak) in which n is 2 or 3 and at least two R¹⁰ groups are ortho to the biaryl bond.

In embodiments, the compound of Formula (Ae), (Af), (Ag), (Ah), (Ai), (Aj) or (Ak) is a compound of Formula (Al) in which at least one R group is OH.

In embodiments, the compound of Formula (A), (Ae), (Af), (Ag), (Ai), (Aj), (Ak) or (Al) is a compound of Formula (Am)

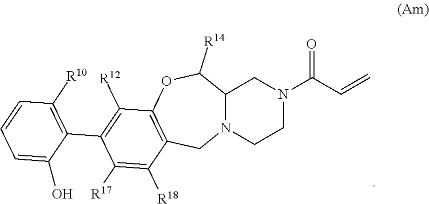

In embodiments, the compound of Formula (A), (Ae), (Af), (Ag), (Ah), (Ai), (Aj), (Ak) or (Al) is a compound of Formula (An) or (Ao)

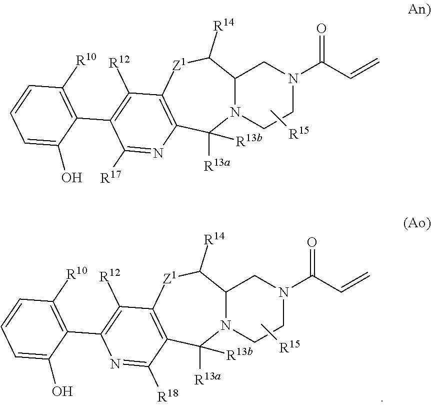

In embodiments, the compound of Formula (A), (Ae), (Af), (Ag), (Ah), (Ai), (Aj), (Ak) or (Al) is a compound of Formula (Ap) in which X¹ and Y¹ are C(O)NR¹⁹.

In embodiments, the compound of Formula (A), (Ae), (Af), (Ag), (Ah), (Ai), (Aj), (Ak) or (Al) is a compound of Formula (Aq) in which X¹ and Y¹ are adjacent ring atoms of an optionally substituted pyrrole, imidazole, pyrazole, 1,2,3-triazole, 1,2,4-triazole, oxazole, isoxazole, 1,2,3-oxadiazole, thiazole or isothiazole.

In embodiments, the compound of Formula (Aq) is a compound of Formula (Ar) in which the optional substituent on the 5-membered ring containing X¹ and Y¹ is selected from C₁-C₃ alkyl, OC₁-C₂ alkyl, OMe, OH, F and Cl.

In embodiments, the compound of Formula (A), (Aa) to (Ar) has the stereochemistry shown below

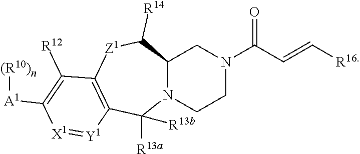

As noted above, in some embodiments the small molecule KRAS inhibitor is a compound of the Formula (III):

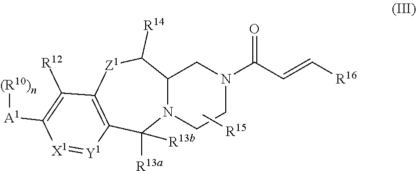

wherein:
A¹ is phenyl or a bicyclic heteroaryl group;
X¹ and Y¹ are connected by a double bond and i) X¹ is CR¹⁷ and Y¹ is CR¹⁸, ii) X¹ is N and Y¹ is CR¹⁸ or iii) X¹ is CR¹⁷ and Y¹ is N; or
X¹ and Y¹ together are C(O)NR¹⁹; or
X¹ and Y¹ are adjacent ring atoms of an optionally substituted 5- or 6-membered N-heterocycle fused to the aromatic ring substituted with Z¹, and X¹ and Y¹ are both C or are C and N;
Z¹ is O, NH, or NMe;
R¹⁰ is independently selected from F, Cl, Br, OH, CH₂OH, OMe, CH₂OMe, C₁-C₃ alkyl, C₁-C₃ fluoroalkyl;
n is 0, 1, 2 or 3;
R¹² is H, F, Cl, CCH, CCMe, CN, Br, C₁-C₃ alkyl, C₁-C₃ fluoroalkyl, OMe or OEt;
R^{13a} and R^{13b} together are ═O or R^{13a} and R^{13b} are H;
R¹⁴ is H or Me;
R¹⁵ is H or Me;
R¹⁶ is H or CH₂NMe₂;
R¹⁷ and R¹⁸ are selected from H, F, Cl, CCH, CN, Me, OH, OMe, O(C₁-C₃ alkyl), C₁-C₃ fluoroalkyl or an optionally substituted 5- or 6-membered carbocycle or heterocycle; or
R¹⁷ and R¹⁸ combine to form an optionally substituted 5- or 6-membered carbocycle or heterocycle;
R¹⁹ is selected from H, Me, Et, C₃H₇ and C₁-C₃ fluoroalkyl;
or a pharmaceutically acceptable salt thereof.

In embodiments, the compound of Formula (III) is a compound of Formula (IIIa) in which R¹⁶ is H.

In embodiments, the compound of Formula (III) or (IIIa), is a compound of Formula (IIIb) in which R¹⁵ is H.

In embodiments, the compound of Formula (III), (IIIa) or (IIIb) is a compound of Formula (IIIc) in which R¹⁴ is H.

In embodiments, the compound of Formula (III), (IIIa), (IIIb) or (IIIc) is a compound of Formula (IIId) in which A¹ is phenyl.

In embodiments, the compound of Formula (III) is a compound of Formula (IIIe)

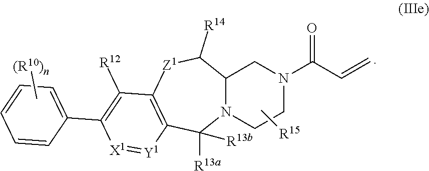

In embodiments, the compound of Formula (IIIe) is a compound of Formula (IIIf) in which R¹⁵ is H.

In embodiments, the compound of Formula (IIIe) or (IIIf) is a compound of Formula (IIIg) in which R^{13a} and R^{13b} are H.

In embodiments, the compound of Formula (IIIe) or (IIIf) is a compound of Formula (IIIh) in which R^{13a} and R^{13b} together are ═O.

In embodiments, the compound of Formula (IIIe), (IIIf), (IIIg) or (IIIh) is a compound of Formula (IIIi) in which Z¹ is O.

In embodiments, the compound of Formula (IIIe), (IIIf), (IIIg), (IIIh) or (IIIi) is a compound of Formula (IIIj) in which R¹² is selected from F or Cl.

In embodiments, the compound of Formula (IIIe), (IIIf), (IIIg), (IIIh), (IIIi) or (IIIj) is a compound of Formula (IIIk) in which n is 2 or 3 and at least two R¹⁰ groups are ortho to the biaryl bond.

In embodiments, the compound of Formula (IIIe), (IIIf), (IIIg), (IIIh), (IIIi), (IIIj) or (IIIk) is a compound of Formula (IIIl) in which at least one R group is OH.

In embodiments, the compound of Formula (III), (IIIe), (IIIf), (IIIg), (IIIi), (IIIj), (IIIk) or (IIIl) is a compound of Formula (IIIm)

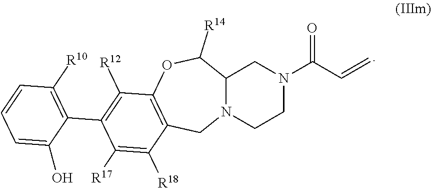

In embodiments, the compound of Formula (III), (IIIe), (IIIf), (IIIg), (IIIh), (IIIi), (IIIj), (IIIk) or (IIIl) is a compound of Formula (IIIn) or (IIIo)

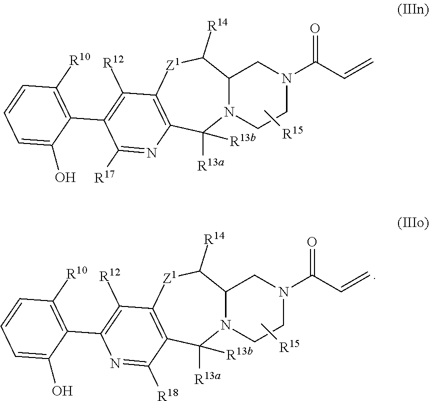

In embodiments, the compound of Formula ((III), (IIIe), (IIIf), (IIIg), (IIIh), (IIIi), (IIIj), (IIIk) or (IIIl) is a compound of Formula (IIIp) in which X¹ and Y¹ are C(O)NR¹⁹.

In embodiments, the compound of Formula (III), (IIIe), (IIIf), (IIIg), (IIIh), (Ii), (IIIj), (IIIk) or (IIIl) is a compound of Formula (IIIq) in which X¹ and Y¹ are adjacent ring atoms of an optionally substituted pyrrole, imidazole, pyrazole, 1,2,3-triazole, 1,2,4-triazole, oxazole, isoxazole, 1,2,3-oxadiazole, thiazole or isothiazole.

In embodiments, the compound of Formula (IIIq) is a compound of Formula (IIIr) in which the optional substituent on the 5-membered ring containing X¹ and Y¹ is selected from C₁-C₃ alkyl, OC₁-C₂ alkyl, OMe, OH, F and Cl.

In embodiments, the compound of Formula (III), (IIIa) to (IIIr) has the stereochemistry shown below

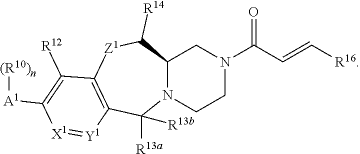

In an embodiment, the compound of Formula (A) is selected from:
(12aS)-2-Acryloyl-10-chloro-9-(5-methyl-1H-indazol-4-yl)-1,2,3,4,12,12a-hexahydro-6H-benzo[f]pyrazino[2,1-c][1,4]oxazepin-6-one;
1-((12aS)-10-Chloro-9-(5-methyl-1H-indazol-4-yl)-3,4,12,12a-tetrahydro-6H-benzo[f]pyrazino[2,1-c][1,4]oxazepin-2(1H)-yl)prop-2-en-1-one;
1-[(12aR)-10-Chloro-9-(2-fluoro-6-hydroxyphenyl)-7-methoxy-3,4,12,12a-tetrahydro-6H-pyrazino[2,1-c][1,4]benzoxazepin-2(1H)-yl]prop-2-en-1-one;
(12aR)-10-chloro-9-(2-fluoro-6-hydroxyphenyl)-7-hydroxy-2-(prop-2-enoyl)-1,2,3,4,12,12a-hexahydro-6H-pyrazino[2,1-c][1,4]benzoxazepin-6-one;
(12aR)-10-Chloro-9-(5-methyl-1H-indazol-4-yl)-2-(prop-2-enoyl)-1,2,3,4,12,12a-hexahydro-6H-pyrazino[2,1-c][1,4]benzoxazepin-6-one;
1-((12aR)-10-Chloro-9-(5-methyl-1H-indazol-4-yl)-3,4,12,12a-tetrahydro-6H-benzo[f]pyrazino[2,1-c][1,4]oxazepin-2(1H)-yl)prop-2-en-1-one;
(12aR)-10-Chloro-8-fluoro-9-(2-fluoro-6-hydroxyphenyl)-2-(prop-2-enoyl)-1,2,3,4,12,12a-hexahydro-6H-pyrazino[2,1-c][1,4]benzoxazepin-6-one;
1-[(12aR)-8,10-dichloro-9-(2-fluoro-6-hydroxyphenyl)-7-hydroxy-3,4,12,12a-tetrahydro-6H-pyrazino[2,1-c][1,4]benzoxazepin-2(1H)-yl]prop-2-en-1-one;
1-[(12aR)-10-Chloro-9-(2-fluoro-6-hydroxyphenyl)-7-(1H-imidazol-1-yl)-3,4,12,12a-tetrahydro-6H-pyrazino[2,1-c][1,4]benzoxazepin-2(1H)-yl]prop-2-en-1-one;
(12aR)-10-Chloro-9-(2-fluoro-6-hydroxyphenyl)-2-(prop-2-enoyl)-1,2,3,4,12,12a-hexahydro-6H-pyrazino[2,1-c][1,4]benzoxazepine-7-carbonitrile;
1-[(12aR)-10-Chloro-9-(2-fluoro-6-hydroxyphenyl)-7-(1H-pyrazol-1-yl)-3,4,12,12a-tetrahydro-6H-pyrazino[2,1-c][1,4]benzoxazepin-2(1H)-yl]prop-2-en-1-one;
1-((12aR)-10-Chloro-8-fluoro-9-(5-methyl-1H-benzo[d]imidazol-4-yl)-3,4,12,12a-tetrahydro-6H-benzo[f]pyrazino[2,1-c][1,4]oxazepin-2(1H)-yl)prop-2-en-1-one;
(12aR)-8,10-Dichloro-9-(2-fluoro-6-hydroxyphenyl)-2-(prop-2-enoyl)-1,2,3,4,12,12a-hexahydro-6H-pyrazino[2,1-c][1,4]benzoxazepin-6-one;
(12aR)-10-Chloro-9-(2-fluoro-6-hydroxyphenyl)-2-(prop-2-enoyl)-1,2,3,4,12,12a-hexahydro-6H-pyrazino[2,1-c][1,4]benzoxazepine-8-carbonitrile;
(12aR)-10-Chloro-9-(2-fluoro-6-hydroxyphenyl)-8-methyl-2-(prop-2-enoyl)-1,2,3,4,12,12a-hexahydro-6H-pyrazino[2,1-c][1,4]benzoxazepin-6-one;
1-[(12aR)-8,10-Dichloro-9-(2-fluoro-6-hydroxyphenyl)-3,4,12,12a-tetrahydro-6H-pyrazino[2,1-c][1,4]benzoxazepin-2(1H)-yl]prop-2-en-1-one;
8-[(12aR)-10-Chloro-8-fluoro-2-(prop-2-enoyl)-1,2,3,4,12,12a-hexahydro-6H-pyrazino[2,1-c][1,4]benzoxazepin-9-yl]-7-methylisoquinolin-1(2H)-one;
1-[(12aR)-10-chloro-9-(2-fluoro-6-hydroxyphenyl)-8-methoxy-3,4,12,12a-tetrahydro-6H-pyrazino[2,1-c][1,4]benzoxazepin-2(1H)-yl]prop-2-en-1-one (12aS)-10-Chloro-9-(5-methyl-1H-indazol-4-yl)-2-(prop-2-enoyl)-1,3,4,11,12,12a-hexahydropyrazino[2,1-c][1,4]benzodiazepin-6(2H)-one;
1-[(12aR)-10-chloro-9-(2-chloro-6-hydroxyphenyl)-8-fluoro-3,4,12,12a-tetrahydro-6H-pyrazino[2,1-c][1,4]benzoxazepin-2(1H)-yl]prop-2-en-1-one;
(12aS)-10-Chloro-11-methyl-9-(5-methyl-1H-indazol-4-yl)-2-(prop-2-enoyl)-1,3,4,11,12,12a-hexahydropyrazino[2,1-c][1,4]benzodiazepin-6(2H)-one;
1-[(12aR)-10-Chloro-9-(2,3-difluoro-6-hydroxyphenyl)-8-fluoro-3,4,12,12a-tetrahydro-6H-pyrazino[2,1-c][1,4]benzoxazepin-2(1H)-yl]prop-2-en-1-one;
(12aR)-10-Chloro-9-(2-hydroxy-6-methylphenyl)-2-(prop-2-enoyl)-1,2,3,4,12,12a-hexahydro-6H-pyrazino[2,1-c][1,4]benzoxazepine-8-carbonitrile;
1-[(12aR)-9-(2-Chloro-6-hydroxyphenyl)-8,10-difluoro-3,4,12,12a-tetrahydro-6H-pyrazino[2,1-c][1,4]benzoxazepin-2(1H)-yl]prop-2-en-1-one;
1-[(12aR)-8,10-Difluoro-9-(2-hydroxy-6-methylphenyl)-3,4,12,12a-tetrahydro-6H-pyrazino[2,1-c][1,4]benzoxazepin-2(1H)-yl]prop-2-en-1-one;
1-[(12aR)-8,10-Difluoro-9-[2-fluoro-6-(hydroxymethyl)phenyl]-3,4,12,12a-tetrahydro-6H-pyrazino[2,1-c][1,4]benzoxazepin-2(1H)-yl]prop-2-en-1-one;
1-[(12aR)-8,10-Difluoro-9-[2-hydroxy-6-(trifluoromethyl)phenyl]-3,4,12,12a-tetrahydro-6H-pyrazino[2,1-c][1,4]benzoxazepin-2(1H)-yl]prop-2-en-1-one;
1-[(12aR)-9-(2-Ethyl-6-hydroxyphenyl)-8,10-difluoro-3,4,12,12a-tetrahydro-6H-pyrazino[2,1-c][1,4]benzoxazepin-2(1H)-yl]prop-2-en-1-one;
1-[(12aR)-9-[2-(Difluoromethyl)-6-hydroxyphenyl]-8,10-difluoro-3,4,12,12a-tetrahydro-6H-pyrazino[2,1-c][1,4]benzoxazepin-2(1H)-yl]prop-2-en-1-one;
(12aR)-9-(2-Chloro-6-hydroxyphenyl)-10-fluoro-2-(prop-2-enoyl)-1,2,3,4,12,12a-hexahydro-6H-pyrazino[2,1-c][1,4]benzoxazepine-8-carbonitrile;
(12aR)-10-Chloro-9-(2-chloro-6-hydroxyphenyl)-2-(prop-2-enoyl)-1,2,3,4,12,12a-hexahydro-6H-pyrazino[2,1-c][1,4]benzoxazepine-8-carbonitrile;
1-[(12aR)-9-(2-Bromo-6-hydroxyphenyl)-8,10-difluoro-3,4,12,12a-tetrahydro-6H-pyrazino[2,1-c][1,4]benzoxazepin-2(1H)-yl]prop-2-en-1-one;
1-[(12aR)-8-Chloro-10-fluoro-9-(2-hydroxy-6-methylphenyl)-3,4,12,12a-tetrahydro-6H-pyrazino[2,1-c][1,4]benzoxazepin-2(1H)-yl]prop-2-en-1-one;
1-[(12aR)-8-Chloro-10-ethynyl-9-(2-hydroxy-6-methylphenyl)-3,4,12,12a-tetrahydro-6H-pyrazino[2,1-c][1,4]benzoxazepin-2(1H)-yl]prop-2-en-1-one;
1-[(12aR)-10-Ethynyl-8-fluoro-9-(2-hydroxy-6-methylphenyl)-3,4,12,12a-tetrahydro-6H-pyrazino[2,1-c][1,4]benzoxazepin-2(1H)-yl]prop-2-en-1-one;
(6aR)-4-Chloro-3-(2-fluoro-6-hydroxyphenyl)-2-methyl-8-(prop-2-enoyl)-2,6,6a,7,8,9,10,12-octahydro-1H-pyrazino[2,1-c]pyrido[3,4-f][1,4]oxazepin-1-one;
1-[(6aR)-1,4-Dichloro-3-(2-fluoro-6-hydroxyphenyl)-6a,7,9,10-tetrahydro-12H-pyrazino[2,1-c]pyrido[3,4-J][1,4]oxazepin-8(6H)-yl]prop-2-en-1-one;
(6aR)-4-Chloro-3-(2-fluoro-6-hydroxyphenyl)-8-(prop-2-enoyl)-2,6,6a,7,8,9,10,12-octahydro-1H-pyrazino[2,1-c]pyrido[3,4-f][1,4]oxazepin-1-one;
1-[(8aR)-6-Chloro-5-(2-fluoro-6-hydroxyphenyl)-8a,9,11,12-tetrahydro-14H-pyrazino[2,1-c][1,2,4]triazolo[4′,3′:1,2]pyrido[3,4-f][1,4]oxazepin-10(8H)-yl]prop-2-en-1-one;
1-[(7aR)-5-chloro-4-(2-fluoro-6-hydroxyphenyl)-1-methyl-1,7a,8,10,11,13-hexahydropyrazino[2′,1′:3,4][1,4]oxazepino[7,6-g]indazol-9(7H)-yl]prop-2-en-1-one;
1-[(7aR)-5-Chloro-4-(2-fluoro-6-hydroxyphenyl)-2-methyl-2,7a,8,10,11,13-hexahydropyrazino[2′,1′:3,4][1,4]oxazepino[7,6-g]indazol-9(7H)-yl]prop-2-en-1-one;
1-[(12aR)-9-(2-chloro-6-hydroxyphenyl)-8-ethynyl-10-fluoro-3,4,12,12a-tetrahydro-6H-pyrazino[2,1-c][1,4]benzoxazepin-2(1H)-yl]prop-2-en-1-one; and
1-[(12aR)-10-Chloro-9-(2-chloro-6-hydroxyphenyl)-8-ethynyl-3,4,12,12a-tetrahydro-6H-pyrazino[2,1-c][1,4]benzoxazepin-2(1H)-yl]prop-2-en-1-one;
1-((12aR)-10-chloro-8-ethynyl-9-(2-fluoro-6-hydroxyphenyl)-3,4,12,12a-tetrahydro-6H-benzo[f]pyrazino[2,1-c][1,4]oxazepin-2(1H)-yl)prop-2-en-1-one;
1-[(7aR)-5-Chloro-4-(2-chloro-6-hydroxyphenyl)-1-methyl-1,7a,8,10,11,13-hexahydroimidazo[4,5-g]pyrazino[2,1-c][1,4]benzoxazepin-9(7H)-yl]prop-2-en-1-one;
1-[(12aR)-8-Chloro-9-(2-chloro-6-hydroxyphenyl)-10-fluoro-3,4,12,12a-tetrahydro-6H-pyrazino[2,1-c][1,4]benzoxazepin-2(1H)-yl]prop-2-en-1-one;
1-[(12aR)-9-(2-Chloro-6-hydroxyphenyl)-10-fluoro-8-(prop-1-yn-1-yl)-3,4,12,12a-tetrahydro-6H-pyrazino[2,1-c][1,4]benzoxazepin-2(1H)-yl]prop-2-en-1-one;
1-[(6aR)-4-Chloro-3-(2-chloro-6-hydroxyphenyl)-2-ethynyl-6a,7,9,10-tetrahydro-12H-pyrazino[2,1-c]pyrido[2,3-][1,4]oxazepin-8(6H)-yl]prop-2-en-1-one;
1-[(12aR)-9-(2-Chloro-6-hydroxyphenyl)-8-ethynyl-10-methyl-3,4,12,12a-tetrahydro-6H-pyrazino[2,1-c][1,4]benzoxazepin-2(1H)-yl]prop-2-en-1-one;
1-[(12aR)-10-chloro-9-(2-chloro-6-hydroxyphenyl)-7,8-difluoro-3,4,12,12a-tetrahydro-6H-pyrazino[2,1-c][1,4]benzoxazepin-2(1H)-yl]prop-2-en-1-one;
1-[(12aR)-9-(2-chloro-6-hydroxyphenyl)-8-(difluoromethoxy)-10-fluoro-3,4,12,12a-tetrahydro-6H-pyrazino[2,1-c][1,4]benzoxazepin-2(1H)-yl]prop-2-en-1-one;
1-[(12aR)-9-(2-chloro-6-hydroxyphenyl)-8-ethynyl-7,10-difluoro-3,4,12,12a-tetrahydro-6H-pyrazino[2,1-c][1,4]benzoxazepin-2(1H)-yl]prop-2-en-1-one;
1-[(12aR)-10-Chloro-9-(2-chloro-6-hydroxyphenyl)-8-(difluoromethoxy)-3,4,12,12a-tetrahydro-6H-pyrazino[2,1-c][1,4]benzoxazepin-2(1H)-yl]prop-2-en-1-one;
1-[(12aR)-9-(2-chloro-6-hydroxyphenyl)-8-(cyclopropyloxy)-10-fluoro-3,4,12,12a-tetrahydro-6H-pyrazino[2,1-c][1,4]benzoxazepin-2(1H)-yl]prop-2-en-1-one;
1-((12aR)-9-(2-Chloro-6-hydroxyphenyl)-8-(3-(dimethylamino)prop-1-yn-1-yl)-10-fluoro-3,4,12,12a-tetrahydro-6H-benzo[f]pyrazino[2,1-c][1,4]oxazepin-2(1H)-yl)prop-2-en-1-one;
1-[(12aR)-9-(2-chloro-6-hydroxyphenyl)-10-fluoro-8-[(pyridin-4-yl)methoxy]-3,4,12,12a-tetrahydro-6H-pyrazino[2,1-c][1,4]benzoxazepin-2(1H)-yl]prop-2-en-1-one;
1-[(12aR)-10-chloro-9-(2-chloro-6-hydroxyphenyl)-8-(2-methoxyethoxy)-3,4,12,12a-tetrahydro-6H-pyrazino[2,1-c][1,4]benzoxazepin-2(1H)-yl]prop-2-en-1-one;
1-[(12aR)-9-(2-Chloro-6-hydroxyphenyl)-10-fluoro-8-[2-(piperidin-1-yl)ethoxy]-3,4,12,12a-tetrahydro-6H-pyrazino[2,1-c][1,4]benzoxazepin-2(1H)-yl]prop-2-en-1-one;
1-[(12aR)-10-Chloro-9-(2-chloro-6-hydroxyphenyl)-8-(prop-1-yn-1-yl)-3,4,12,12a-tetrahydro-6H-pyrazino[2,1-c][1,4]benzoxazepin-2(1H)-yl]prop-2-en-1-one;
1-[(6aR)-4-Chloro-3-(2-chloro-6-hydroxyphenyl)-2-[(²H₃)methyloxy]-6a,7,9,10-tetrahydro-12H-pyrazino[2,1-c]pyrido[2,3-][1,4]oxazepin-8(6H)-yl]prop-2-en-1-one;
1-[(12aR)-10-Chloro-9-(2-chloro-6-hydroxyphenyl)-8-(methoxymethyl)-3,4,12,12a-tetrahydro-6H-pyrazino[2,1-c][1,4]benzoxazepin-2(1H)-yl]prop-2-en-1-one; and
1-[(12aR)-9-(2-Chloro-6-hydroxyphenyl)-7-[2-(dimethylamino)ethoxy]-10-fluoro-3,4,12,12a-tetrahydro-6H-pyrazino[2,1-c][1,4]benzoxazepin-2(1H)-yl]prop-2-en-1-one;
1-[(6aR)-4-Chloro-3-(2-chloro-6-hydroxyphenyl)-1-(prop-1-yn-1-yl)-6a,7,9,10-tetrahydro-12H-pyrazino[2,1-c]pyrido[3,4-f][1,4]oxazepin-8(6H)-yl]prop-2-en-1-one; and
1-((6aR)-4-Chloro-3-(2-chloro-6-hydroxyphenyl)-1-ethynyl-6a,7,9,10-tetrahydro-12H-pyrazino[2,1-c]pyrido[3,4-f][1,4]oxazepin-8(6H)-yl)prop-2-en-1-one;
or a pharmaceutically acceptable salt thereof.

As will be appreciated by the person of skill in the art, the compounds of Formula (A) contain a biaryl bond between A¹ and the ring containing X¹ and Y¹.

Statements relating to compounds of Formula (III) herein below, should be read to apply equally to compounds of Formula (A).

The compounds of Formula (A), for example compounds of Formula (III), have one or more chiral centers, for example at the bridgehead carbon between the piperazine to which the acrylamide is attached and the ring comprising Z¹, and it will be recognized that the compound of Formula (A), for example compounds of Formula (III), may be prepared, isolated and/or supplied with or without the presence, in addition, of one or more of the other possible stereoisomeric forms of the compound of Formula (A) in any relative proportions. The preparation of stereoenriched or stereopure compounds may be carried out by standard techniques of organic chemistry that are well known in the art, for example by synthesis from stereoenriched or stereopure starting materials, use of an appropriate stereoenriched or stereopure catalysts during synthesis, and/or by resolution of a racemic or partially enriched mixture of stereoisomers, for example via chiral chromatography. In preferred embodiments, the compounds of the present specification are in the (R)-configuration when Z¹ is O as shown below.

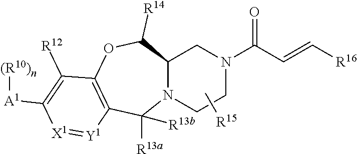

In particular, the compounds of Formula (III) may possess axial chirality, by virtue of restricted rotation around the biaryl bond between A¹ and the ring containing X¹ and Y¹ and as such may exist as mixtures of atropisomers with enantiomeric excess between about 0% and >98% ee. When a compound is a pure atropisomer, the stereochemistry at each chiral center may be specified by either aR or aS. Such designations may also be used for mixtures that are enriched in one atropisomer. By way of example only, the following moiety may exhibit atropisomerism and be capable of resolution into the aR and aS atropisomers by chiral chromatography. For illustration, the two atropisomers of a compound of Formula (III) in which A¹ is 2-F, 6-hydroxyphenyl are shown below (R¹³, R¹⁴, R¹⁵ and R¹⁶ are omitted for clarity).

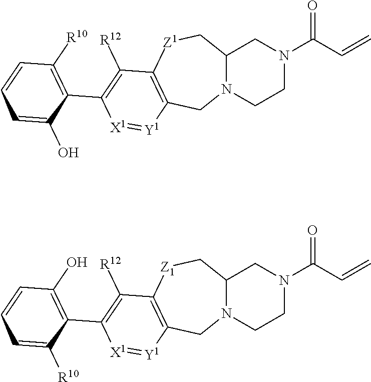

Further description of atropisomerism and axial chirality and rules for assignment of configuration can be found in Eliel, E. L. & Wilen, S. H. ‘Stereochemistry of Organic Compounds’ John Wiley and Sons, Inc. 1994. In the compounds of the specification the groups R¹⁰, R¹² and X¹ may be selected to eliminate or substantially reduce the interconversion between the (aR) and (aS) atropisomers.

In more detail, the interaction between the group(s) A¹(R¹⁰)ₙ and the substituent R¹² and/or X¹ may advantageously restrict the rotation around the bond between the ring A¹ and the ring containing X¹. The interaction between the substituent R¹² and the ring A¹ and/or the substituent(s) R¹⁰ thereon may as a result be used to stabilize atropisomers of the compounds according to the present specification. This in turn may advantageously allow isolation of a stable atropisomer that exhibits higher activity as an inhibitor of G12C mutated Ras than the second atropisomer. It will be understood that the more active atropisomers are preferred embodiments.

In embodiments of the compound of Formula (III) wherein the group X¹ is substituted, that substituent may, like R¹², be capable of stabilizing atropisomers of the compounds according to the specification.

Pharmaceutical compositions which comprise a compound of the Formula (III) or a pharmaceutically acceptable salt thereof, in association with a pharmaceutically acceptable excipient, optionally further comprise one or more of the other stereoisomeric forms of the compound of Formula (III) or a pharmaceutically acceptable salt thereof, wherein the compound of Formula (III) or pharmaceutically acceptable salt thereof is present within the composition with a diastereomeric excess (% de.) of ≥90%.

Pharmaceutical compositions which comprise a compound of the Formula (III) or a pharmaceutically acceptable salt thereof, in association with a pharmaceutically acceptable excipient, optionally further comprise one or more of the other stereoisomeric forms of the compound of Formula (III) or a pharmaceutically acceptable salt thereof, wherein the compound of Formula (III) or pharmaceutically acceptable salt thereof is present within the composition with an enantiomeric excess (% ee) of ≥90% and a diastereomeric excess (% de) of ≥90%.

The compounds of Formula (III) and pharmaceutically acceptable salts thereof may be prepared, used or supplied in amorphous form, crystalline form, or semi-crystalline form and any given compound of Formula (III) or pharmaceutically acceptable salt thereof may be capable of being formed into more than one crystalline/polymorphic form, including hydrated (e.g. hemi-hydrate, a mono-hydrate, a di-hydrate, a tri-hydrate or other stoichiometry of hydrate) and/or solvated forms. It is to be understood that the present specification encompasses any and all such solid forms of the compound of Formula (III) and pharmaceutically acceptable salts thereof.

In some embodiments, the small molecule KRAS inhibitor is a compound of Formula (IV):

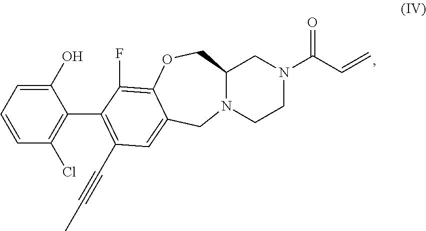 [SMILES: C=CC(=O)N1CCN2Cc3cc(C#CC)c(-c4c(O)cccc4Cl)c(F)c3OC[C@H]2C1]

which is 1-[(12aR)-9-(2-chloro-6-hydroxyphenyl)-10-fluoro-8-(prop-1-yn-1-yl)-3,4,12,12a-tetrahydro-6H-pyrazino[2,1-c][1,4]benzoxazepin-2(1H)-yl]prop-2-en-1-one, or a pharmaceutically acceptable salt thereof.

Ataxia-telangiectasia mutated and RAD-3 related protein kinase, i.e., ATR protein kinase (also known as FRAP-Related Protein 1; FRP1; MEC1; SCKL; SECKL1), is a member of the PI3-Kinase like kinase (PIKK) family of proteins that are involved in repair and maintenance of the genome and its stability (reviewed in Cimprich K. A. and Cortez D. 2008, Nature Rev. Mol. Cell Biol. 9:616-627). The present disclosure provides for administering inhibitors of ATR.

In some embodiments, the ATR inhibitor can inhibit human ATR. As described previously, the qualifier “human” as used herein in connection with an ATR protein may in a certain interpretation refer to the amino acid sequence of the ATR protein, but includes ATR protein found in humans obtained by other technical means, e.g., by recombinant expression, cell-free translation, or non-biological peptide synthesis.

Various compounds that inhibit ATR are known in the art and can include antibodies that specifically bind ATR. In some embodiments, the ATR inhibitor is an antibody or other therapeutic protein which can selectively bind to ATR. In some embodiments, the ATR inhibitor is an antibody.

In some embodiments, the ATR inhibitor is a small molecule ATR inhibitor. The term “small molecule” ATR inhibitor, as used herein refers non-peptide based and non-nucleic acid based chemical compounds which reduce the activity of ATR. In some embodiments, the small molecule ATR inhibitor reduces the activity of ATR. In some embodiments, the small molecule ATR inhibitor increases the degradation of ATR, thereby decreasing the activity of ATR function. In some embodiments, the small molecule ATR inhibitor has a molecular weight of less than 1.5 kDa, less than 1.0 kDa, or less than 700 kDa. In some embodiments, the small molecule ATR inhibitor binds to ATR. In some embodiments, the small molecule ATR inhibitor has an IC50 of less than about 1 mM, less than about 100 μM, less than about 50 μM, less than about 25 μM, 10 μM, less than about 1 μM, less than about 500 nM, less than about 100 nM, less than about 50 nM, less than about 25 nM, less than about 10 nM, less than about 5 nM, or less than about 2.5 nM. In some embodiments, the small molecule ATR inhibitor has an IC50 of less than about 2 nM or less about 1 nM. In some embodiments, the small molecule ATR inhibitor has an IC50 of about 0.05 nM to about 50 nM, about 0.1 nM to about 10 nM, about 0.1 nM to about 25 nM, or about 0.5 nM to about 10 nM. In some embodiments, the small molecule ATR inhibitor has an IC 50 of about 0.5 nM to about 5 nM, or about 0.5 nM to about 2 nM.

For example, in some embodiments the small molecule ATR inhibitor is M6620/VX970 (berzosertib; Merck KGaA, Formula B),

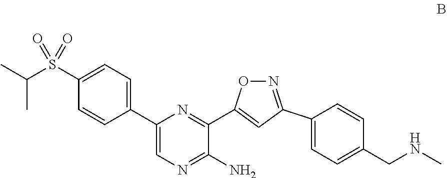 [SMILES: CNCc1ccc(-c2cc(-c3nc(-c4ccc(S(=O)(=O)C(C)C)cc4)cnc3N)on2)cc1]

BAY-1895344 (elimusertib, Bayer KGaA, Formula C),

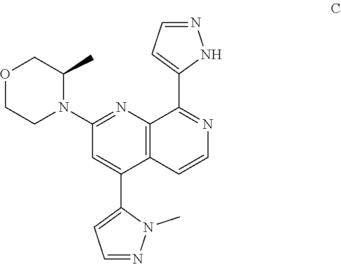 [SMILES: C[C@@H]1COCCN1c1cc(-c2ccnn2C)c2ccnc(-c3ccn[nH]3)c2n1]

RP-3500 (camonsertib, Roche, Formula D)

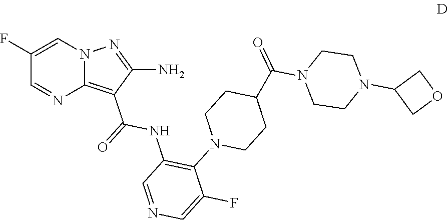 [SMILES: Nc1nn2cc(F)cnc2c1C(=O)Nc1cncc(F)c1N1CCC(C(=O)N2CCN(C3COC3)CC2)CC1]

ATRN-119 (Aprea Therapeutics), SC0245 (Shijiazhuang Sagacity New Drug Development Co Ltd/WuXi AppTec Co), ATRN-212 (Aprea Therapeutics), LR-02 (Laevoroc Oncology AG), M4344 (Gartisertib. Merck KGaA, Formula E),

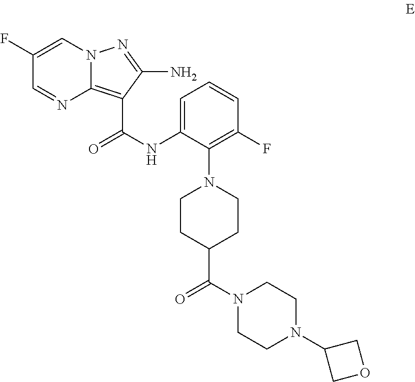 [SMILES: Nc1nn2cc(F)cnc2c1C(=O)Nc1cccc(F)c1N1CCC(C(=O)N2CCN(C3COC3)CC2)CC1]

### M1774 (Tuvusertib, Merck KGaA, Formula F)

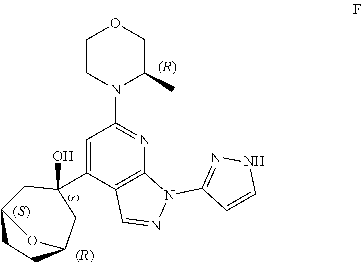

### ATG-018 (Antengene Corp Ltd), ART-0380 (Artios), BG-129 (Celator Pharmaceuticals), JS-123 (Shanghai Junshi Biosciences Co Ltd; Wigen Biomedicine Technology (Shanghai) Co Ltd), BKT-300 (Biokine Therapeutics Ltd, Formula G)

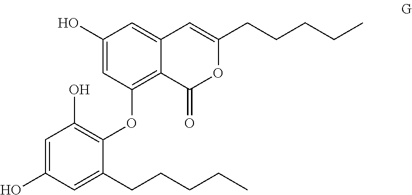 [SMILES: CCCCCc1cc2cc(O)cc(Oc3c(O)cc(O)cc3CCCCC)c2c(=O)o1]

### AZ-20 (AstraZeneca, Formula H)

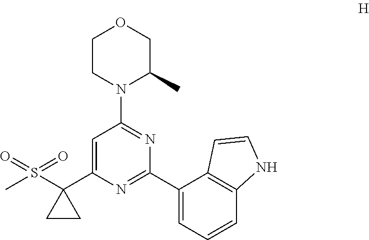 [SMILES: C[C@@H]1COCCN1c1cc(C2(S(C)(=O)=O)CC2)nc(-c2cccc3[nH]ccc23)n1]

### VE-821 (University of Oxford, Formula J)

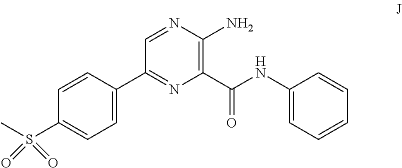 [SMILES: CS(=O)(=O)c1ccc(-c2cnc(N)c(C(=O)Nc3ccccc3)n2)cc1]

### AZD-5597 (The University of Newcastle Upon Tyne, Formula K)

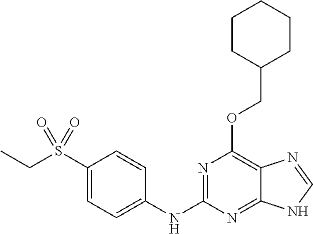 [SMILES: CCS(=O)(=O)c1ccc(Nc2nc(OCC3CCCCC3)c3nc[nH]c3n2)cc1]

### or IMP-9064 (Impact Therapeutics).

Small molecule ATR compounds of Formula (V) are described in WO2011154737, the content of which is hereby incorporated by reference in its entirety. In some embodiments, the small molecule ATR inhibitor is a compound of Formula (V):

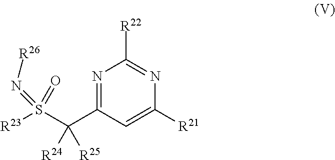

wherein:
R²¹ is selected from morpholin-4-yl and 3-methylmorpholin-4-yl;
R²² is

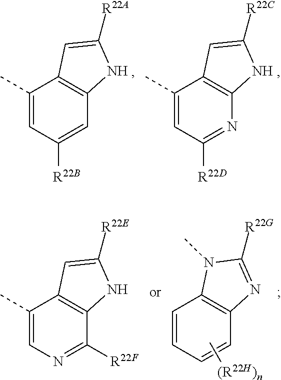

n is 0 or 1;
R^{22A}, R^{22C}, R^{22E} and R^{22F} each independently are hydrogen or methyl;
R^{22B} and R^{22D} each independently are hydrogen or methyl;
R^{22G} is selected from —NHR²⁷ and —NHCOR²⁸;
R^{22H} is fluoro;
R²³ is methyl;
R²⁴ and R²⁵ are each independently hydrogen or methyl, or R²⁴ and R²⁵ together with the atom to which they are attached form Ring A²;
Ring A² is a C₃₋₆ cycloalkyl or a saturated 4-6 membered heterocyclic ring containing one heteroatom selected from O and N;
R²⁶ is hydrogen;
R²⁷ is hydrogen or methyl; and
R²⁸ is methyl, or a pharmaceutically acceptable salt thereof.

In accordance with a further aspect of the present disclosure, the small molecule ATR inhibitor is a compound of Formula (V):

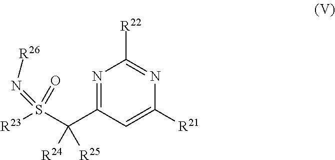

wherein:
R²¹ is 3-methylmorpholin-4-yl;
R²² is

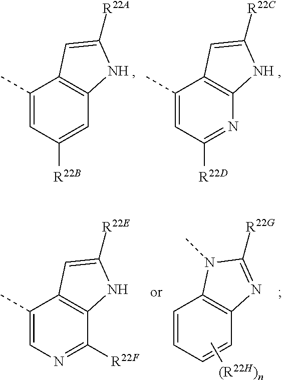

n is 0 or 1;

R^{22A}, R^{22C}, R^{22E} and R^{22F} each independently are hydrogen or methyl;
R^{22B} and R^{22D} each independently are hydrogen or methyl;
R^{22G} is selected from —NH₂, —NHMe and —NHCOMe;
R^{22H} is fluoro;
R²³ is methyl;
R²⁴ and R²⁵ are each independently hydrogen or methyl, or R²⁴ and R²⁵ together with the atom to which they are attached form Ring A²;
Ring A² is a C₃₋₆ cycloalkyl or a saturated 4-6 membered heterocyclic ring containing one heteroatom selected from O and N; and
R²⁶ is hydrogen, or a pharmaceutically acceptable salt thereof.

Certain compounds of Formula (V) are capable of existing in stereoisomeric forms. It will be understood that the disclosure encompasses all geometric and optical isomers of the compounds of Formula (V) and mixtures thereof including racemates. Tautomers and mixtures thereof also form an aspect of the present disclosure. Solvates and mixtures thereof also form an aspect of the present disclosure. For example, a suitable solvate of a compound of Formula (V) is, for example, a hydrate such as a hemi-hydrate, a mono-hydrate, a di-hydrate or a tri-hydrate or an alternative quantity thereof.

It is to be understood that, insofar as certain of the compounds of Formula (V) defined above may exist in optically active or racemic forms by virtue of one or more asymmetric carbon atoms or sulphur atoms, the disclosure includes in its definition any such optically active or racemic form which possesses the above-mentioned activity. The present disclosure encompasses all such stereoisomers having activity as herein defined. It is further to be understood that in the names of chiral compounds (R,S) denotes any scalemic or racemic mixture while (R) and (S) denote the enantiomers. In the absence of (R,S), (R) or (S) in the name it is to be understood that the name refers to any scalemic or racemic mixture, wherein a scalemic mixture contains R and S enantiomers in any relative proportions and a racemic mixture contains R and S enantiomers in the ratio 50:50. The synthesis of optically active forms may be carried out by standard techniques of organic chemistry well known in the art, for example by synthesis from optically active starting materials or by resolution of a racemic form. Racemates may be separated into individual enantiomers using known procedures (see, for example, Advanced Organic Chemistry: 3rd Edition: author J March, p 104-107). A suitable procedure involves formation of diastereomeric derivatives by reaction of the racemic material with a chiral auxiliary, followed by separation, for example by chromatography, of the diastereomers and then cleavage of the auxiliary species. Similarly, the above-mentioned activity may be evaluated using the standard laboratory techniques referred to hereinafter.

It will be understood that the disclosure encompasses compounds with one or more isotopic substitutions. For example, H may be in any isotopic form, including ¹H, ²H (D), and ³H (T); C may be in any isotopic form, including ¹²C, ¹³C, and ¹⁴C; O may be in any isotopic form, including ¹⁶O and ¹⁸O; and the like.

The present disclosure relates to the compounds of Formula (V) as herein defined as well as to salts thereof. Salts for use in pharmaceutical compositions will be pharmaceutically acceptable salts, but other salts may be useful in the production of the compounds of Formula (V) and their pharmaceutically acceptable salts. Pharmaceutically acceptable salts of the disclosure may, for example, include acid addition salts of compounds of Formula (V) as herein defined which are sufficiently basic to form such salts. Such acid addition salts include but are not limited to furmarate, methanesulfonate, hydrochloride, hydrobromide, citrate and maleate salts and salts formed with phosphoric and sulfuric acid. In addition where compounds of Formula (V) are sufficiently acidic, salts are base salts and examples include but are not limited to, an alkali metal salt for example sodium or potassium, an alkaline earth metal salt for example calcium or magnesium, or organic amine salt for example triethylamine, ethanolamine, diethanolamine, triethanolamine, morpholine, N-methylpiperidine, N-ethylpiperidine, dibenzylamine or amino acids such as lysine.

The compounds of Formula (V) may also be provided as in vivo hydrolysable esters. An in vivo hydrolysable ester of a compound of Formula (V) containing carboxy or hydroxy group is, for example, a pharmaceutically acceptable ester which is cleaved in the human or animal body to produce the parent acid or alcohol. Such esters can be identified by administering, for example, intravenously to a test animal, the compound under test and subsequently examining the test animal's body fluid.

Suitable pharmaceutically acceptable esters for carboxy include C₁₋₆ alkoxymethyl esters for example methoxymethyl, C₁₋₆ alkanoyloxymethyl esters for example pivaloyloxymethyl, phthalidyl esters, C₃₋₈ cycloalkoxycarbonyloxyC₁₋₆alkyl esters for example 1-cyclohexylcarbonyloxyethyl, 1,3-dioxolen-2-onylmethyl esters for example 5-methyl-1,3-dioxolen-2-onylmethyl, and C₁₋₆ alkoxycarbonyloxyethyl esters for example 1-methoxycarbonyloxyethyl; and may be formed at any carboxy group in the compounds of this disclosure.

Suitable pharmaceutically acceptable esters for hydroxy include inorganic esters such as phosphate esters (including phosphoramidic cyclic esters) and α-acyloxyalkyl ethers and related compounds which as a result of the in vivo hydrolysis of the ester breakdown to give the parent hydroxy group/s. Examples of α-acyloxyalkyl ethers include acetoxymethoxy and 2,2-dimethylpropionyloxymethoxy. A selection of in vivo hydrolysable ester forming groups for hydroxy include C₁₋₁₀ alkanoyl, for example formyl, acetyl, benzoyl, phenylacetyl, substituted benzoyl and phenylacetyl; C₁₋₁₀ alkoxycarbonyl (to give alkyl carbonate esters), for example ethoxycarbonyl; di-C₁₋₄ alkylcarbamoyl and N-(di-C₁₋₄ alkylaminoethyl)-N—C₁₋₄ alkylcarbamoyl (to give carbamates); di-C₁₋₄ alkylaminoacetyl and carboxyacetyl. Examples of ring substituents on phenylacetyl and benzoyl include aminomethyl, C₁₋₄ alkylaminomethyl and di-(C₁₋₄ alkyl)aminomethyl, and morpholino or piperazino linked from a ring nitrogen atom via a methylene linking group to the 3- or 4-position of the benzoyl ring. Other interesting in vivo hydrolysable esters include, for example, R^{A}C(O)OC₁₋₆ alkyl-CO—, wherein R^{A} is for example, benzyloxy-C₁₋₄ alkyl, or phenyl. Suitable substituents on a phenyl group in such esters include, for example, 4-C₁₋₄ piperazino-C₁₋₄ alkyl, piperazino-C₁₋₄ alkyl and morpholino-C₁₋₄ alkyl.

The compounds of Formula (V) can be administered in the form of a prodrug which is broken down in the human or animal body to give a compound of Formula (V). Various forms of prodrugs are known in the art. For examples of such prodrug derivatives, see:
a) Design of Prodrugs, edited by H. Bundgaard, (Elsevier, 1985) and Methods in Enzymology, Vol. 42, p. 309-396, edited by K. Widder, et al. (Academic Press, 1985);
b) A Textbook of Drug Design and Development, edited by Krogsgaard-Larsen and H. Bundgaard, Chapter 5 “Design and Application of Prodrugs”, by H. Bundgaard p. 113-191 (1991);
c) H. Bundgaard, Advanced Drug Delivery Reviews, 8, 1-38 (1992);
d) H. Bundgaard, et al., Journal of Pharmaceutical Sciences, 77, 285 (1988); and
e) N. Kakeya, et al., Chem Pharm Bull, 32, 692 (1984).

Particular values of Ring A², n (as it relates to Formula (V)), R²¹, R²², R²⁴, R²⁵, R²⁶, R²⁷ and R²⁸ are as follows. Such values may be used individually or in combination where appropriate, in connection with any aspect of the disclosure, or part thereof, and with any of the definitions, claims or embodiments defined herein.
n: In one aspect, n is 0. In another aspect, n is 1.
R²¹: In one aspect, R²¹ is selected from morpholin-4-yl and 3-methylmorpholin-4-yl. In a further aspect, R²¹ is 3-methylmorpholin-4-yl. In a further aspect, R²¹ is

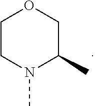

In a further aspect, R²¹ is

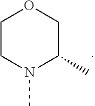

R²²: In one aspect, R²² is

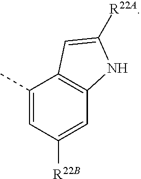

In one aspect, R²² is

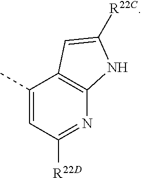

In one aspect, R²² is

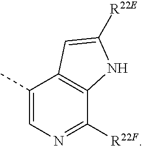

In one aspect, R²² is

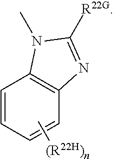

R^{22A}: In one aspect, R^{22A} is hydrogen.
R^{22B}: In one aspect, R^{22B} is hydrogen.
R^{22C}: In one aspect, R^{22C} is hydrogen.
R^{22D}: In one aspect, R^{22D} is hydrogen.
R^{22E}: In one aspect, R^{22E} is hydrogen.
R^{22F}: In one aspect, R^{22F} is hydrogen.
R^{22G}: In one aspect of the disclosure, R^{22G} is selected from —NHR²⁷ and —NHCOR²⁸. In one aspect of the disclosure, R^{22G} is —NHR²⁷. In one aspect of the disclosure, R^{22G} is —NHCOR²⁸. In one aspect of the disclosure, R^{22G} is selected from —NH₂, —NHMe and —NHCOMe. In one aspect of the disclosure, R^{22G} is —NH₂. In one aspect of the disclosure, R^{22G} is —NHMe. In one aspect of the disclosure, R^{22G} is —NHCOMe.

R²⁴ and R²⁵: In one aspect of the disclosure, R²⁴ and R²⁵ are hydrogen. In one aspect of the disclosure, R²⁴ and R²⁵ are methyl. In one aspect of the disclosure, R²⁴ and R²⁵ together with the atom to which they are attached form Ring A².

Ring A²: In one aspect of the disclosure, Ring A² is a C₃₋₆ cycloalkyl or a saturated 4-6 heterocyclic ring containing one heteroatom selected from O and N. In another aspect, Ring A² is a cyclopropyl, cyclobutyl, cyclopentyl, oxetanyl, tetrahydrofuryl, tetrahydropyranyl, azetidinyl, pyrrolidinyl or piperidinyl ring. In another aspect, Ring A² is a cyclopropyl, cyclobutyl, cyclopentyl, tetrahydropyranyl or piperidinyl ring. In another aspect, Ring A² is a cyclopropyl, cyclopentyl, tetrahydropyranyl or piperidinyl ring. In another aspect, Ring A² is a cyclopropyl, tetrahydropyranyl or piperidinyl ring. In another aspect, Ring A² is a cyclopropyl or tetrahydropyranyl ring. In another aspect, Ring A² is a piperidinyl ring. In another aspect, Ring A² is a tetrahydropyranyl ring. In another aspect, Ring A² is a cyclopropyl ring.
R²⁶: In one aspect, R²⁶ is hydrogen.
R²⁷: In one aspect, R²⁷ is hydrogen or methyl. In one aspect, R²⁷ is methyl. In one aspect, R²⁷ is hydrogen.
R²⁸: In one aspect, R²⁸ is methyl.

In one aspect, the small molecule ATR inhibitor is a compound of Formula (V), or a pharmaceutically acceptable salt thereof, in which R²¹ is selected from morpholin-4-yl and 3-methylmorpholin-4-yl; n is 0 or 1; R^{22A} is hydrogen; R^{22B} is hydrogen; R^{22C} is hydrogen; R^{22D} is hydrogen; R^{22E} is hydrogen; R^{22F} is hydrogen; R^{22G} is selected from —NHR²⁷ and —NHCOR²⁸; R^{22H} is fluoro; R²³ is methyl; R²⁴ and R²⁵ together with the atom to which they are attached form Ring A²; Ring A² is a C₃₋₆ cycloalkyl or a saturated 4-6 heterocyclic ring containing one heteroatom selected from O and N; R²⁶ is hydrogen; R²⁷ is hydrogen or methyl; and R²⁸ is methyl.

In another aspect, the small molecule ATR inhibitor is a compound of Formula (V), or a pharmaceutically acceptable salt thereof, in which R²¹ is selected from morpholin-4-yl and 3-methylmorpholin-4-yl; n is 0 or 1; R^{22A} is hydrogen; R²²3 is hydrogen; R^{22C} is hydrogen; R^{22D} is hydrogen; R^{22E} is hydrogen; R^{22F} is hydrogen; R^{22G} is selected from —NH₂, —NHMe and —NHCOMe; R^{22H} is fluoro; R²³ is methyl; R²⁴ and R²⁵ together with the atom to which they are attached form Ring A²; Ring A² is a C₃₋₆ cycloalkyl or a saturated 4-6 heterocyclic ring containing one heteroatom selected from O and N; and R²⁶ is hydrogen.

In another aspect, the small molecule ATR inhibitor is a compound of Formula (V), or a pharmaceutically acceptable salt thereof, in which R²¹ is selected from morpholin-4-yl and 3-methylmorpholin-4-yl; n is 0 or 1; R^{22A} is hydrogen; R²²3 is hydrogen; R^{22C} is hydrogen; R^{22D} is hydrogen; R^{22E} is hydrogen; R^{22F} is hydrogen; R^{22G} is selected from —NHR²⁷ and —NHCOR²⁸; R^{22H} is fluoro; R²³ is methyl; R²⁴ and R²⁵ together with the atom to which they are attached form Ring A²; Ring A² is a cyclopropyl, cyclobutyl, cyclopentyl, oxetanyl, tetrahydrofuryl, tetrahydropyranyl, azetidinyl, pyrrolidinyl or piperidinyl ring; R²⁶ is hydrogen; R²⁷ is hydrogen or methyl; and R²⁸ is methyl.

In another aspect, the small molecule ATR inhibitor is a compound of Formula (V), or a pharmaceutically acceptable salt thereof, in which R²¹ is selected from morpholin-4-yl and 3-methylmorpholin-4-yl; n is 0 or 1; R^{22A} is hydrogen; R^{22E} is hydrogen; R^{22C} is hydrogen; R^{22D} is hydrogen; R^{22E} is hydrogen; R^{22F} is hydrogen; R^{22G} is selected from —NH₂, —NHMe and —NHCOMe; R^{22H} is fluoro; R²³ is methyl; R²⁴ and R⁵ together with the atom to which they are attached form Ring A²; Ring A² is a cyclopropyl, cyclobutyl, cyclopentyl, oxetanyl, tetrahydrofuryl, tetrahydropyranyl, azetidinyl, pyrrolidinyl or piperidinyl ring; and R²⁶ is hydrogen.

In another aspect, the small molecule ATR inhibitor is a compound of Formula (Va),

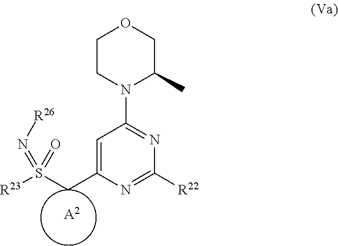

or a pharmaceutically acceptable salt thereof; wherein:
Ring A² is a cyclopropyl, tetrahydropyranyl or piperidinyl ring;
R²² is

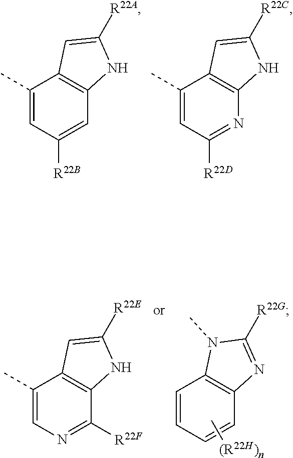

n is 0 or 1; R^{22A} is hydrogen; R^{22B} is hydrogen; R^{22C} is hydrogen; R^{22D} is hydrogen; R^{22E} is hydrogen; R^{22F} is hydrogen; R^{22G} is selected from —NHR²⁷ and —NHCOR²⁸; R^{22H} is fluoro; R²³ is a methyl group; R²⁶ is hydrogen; R²⁷ is hydrogen or methyl; and R²⁸ is methyl.

In another aspect, the small molecule ATR inhibitor is a compound of Formula (Va),

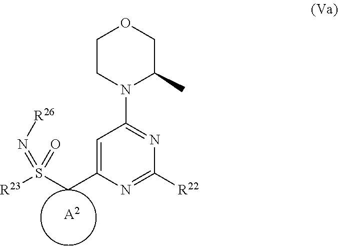

or a pharmaceutically acceptable salt thereof; wherein:
Ring A² is a cyclopropyl, tetrahydropyranyl or piperidinyl ring;
R²² is

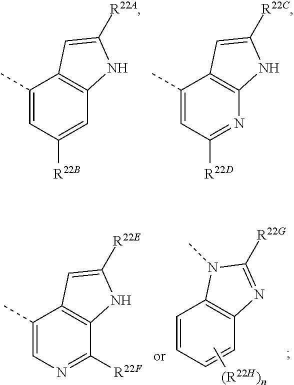

n is 0 or 1; R^{22A} is hydrogen; R^{22B} is hydrogen; R^{22C} is hydrogen; R^{22D} is hydrogen; R^{22E} is hydrogen; R^{22F} is hydrogen; R^{22G} is selected from —NH₂, —NHMe and —NHCOMe; R^{22H} is fluoro; R²³ is a methyl group; and R²⁶ is hydrogen.

In another aspect, the small molecule ATR inhibitor is a compound of Formula (Va),

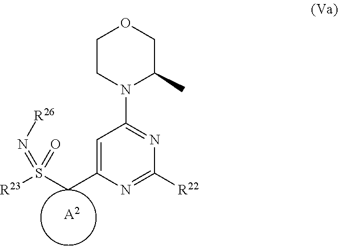

or a pharmaceutically acceptable salt thereof; wherein:
Ring A² is a cyclopropyl, tetrahydropyranyl or piperidinyl ring;
R²² is

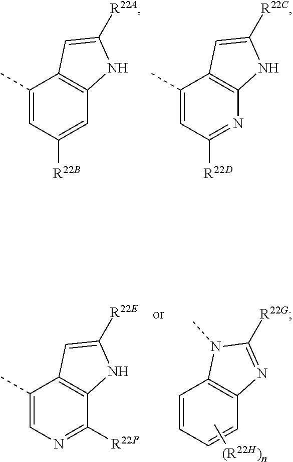

n is 0 or 1; R^{22A} is hydrogen; R^{22B} is hydrogen; R^{22C} is hydrogen; R^{22D} is hydrogen; R^{22E} is hydrogen; R^{22F} is hydrogen; R^{22G} is —NHR²⁷; R^{22H} is fluoro; R²³ is a methyl group; R²⁶ is hydrogen; and R²⁷ is hydrogen.

In another aspect, the small molecule ATR inhibitor is a compound of Formula (Va),

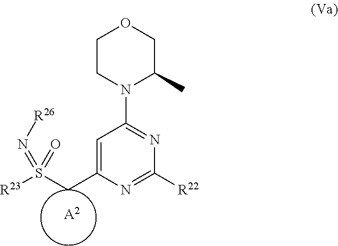

or a pharmaceutically acceptable salt thereof; wherein:
Ring A² is a cyclopropyl ring;
R²² is

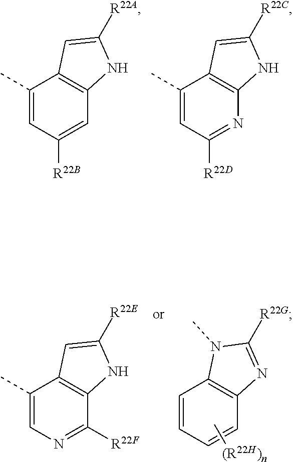

n is 0; R^{22A} is hydrogen; R^{22B} is hydrogen; R^{22C} is hydrogen; R^{22D} is hydrogen; R^{22E} is hydrogen; R^{22F} is hydrogen; R^{22G} is —NHR²⁷; R^{22H} is fluoro; R²³ is a methyl group; R²⁶ is hydrogen; and R²⁷ is methyl.

In another aspect, the small molecule ATR inhibitor is a compound selected from any one of
4-{4-[(3R)-3-Methylmorpholin-4-yl]-6-[((R)—S-methylsulfonimidoyl)methyl]pyrimidin-2-yl}-1H-pyrrolo[2,3-b]pyridine;
4-{4-[(3R)-3-Methylmorpholin-4-yl]-6-[1-((S)—S-methylsulfonimidoyl)cyclopropyl]pyrimidin-2-yl}-1H-pyrrolo[2,3-b]pyridine;
4-{4-[(3R)-3-Methylmorpholin-4-yl]-6-[1-((R)—S-methylsulfonimidoyl)cyclopropyl]pyrimidin-2-yl}-1H-pyrrolo[2,3-b]pyridine;
N-Methyl-1-{4-[(3R)-3-methylmorpholin-4-yl]-6-[1-((R)—S-methylsulfonimidoyl)cyclopropyl]pyrimidin-2-yl}-1H-benzimidazol-2-amine;
N-Methyl-1-{4-[(3R)-3-methylmorpholin-4-yl]-6-[1-((S)—S-methylsulfonimidoyl)cyclopropyl]pyrimidin-2-yl}-1H-benzimidazol-2-amine;
4-{4-[(3R)-3-Methylmorpholin-4-yl]-6-[1-((R)—S-methylsulfonimidoyl)cyclopropyl]pyrimidin-2-yl}-1H-indole;
4-{4-[(3R)-3-methylmorpholin-4-yl]-6-[1-((S)—S-methylsulfonimidoyl)cyclopropyl]pyrimidin-2-yl}-1H-indole;
1-{4-[(3R)-3-Methylmorpholin-4-yl]-6-[1-((R)—S-methylsulfonimidoyl)cyclopropyl]pyrimidin-2-yl}-1H-benzimidazol-2-amine;
1-{4-[(3R)-3-methylmorpholin-4-yl]-6-[1-((S)—S-methylsulfonimidoyl)cyclopropyl]pyrimidin-2-yl}-1H-benzimidazol-2-amine;
4-Fluoro-N-methyl-1-{4-[(3R)-3-methylmorpholin-4-yl]-6-[1-((R)—S-methylsulfonimidoyl)cyclopropyl]pyrimidin-2-yl}-1H-benzimidazol-2-amine;
4-fluoro-N-methyl-1-{4-[(3R)-3-methylmorpholin-4-yl]-6-[1-((S)—S-methylsulfonimidoyl)cyclopropyl]pyrimidin-2-yl}-1H-benzimidazol-2-amine;
4-{4-[(3R)-3-Methylmorpholin-4-yl]-6-[1-(S-methylsulfonimidoyl)cyclopropyl]pyrimidin-2-yl}-1H-pyrrolo[2,3-c]pyridine;
N-methyl-1-{4-[1-methyl-1-((S)—S-methylsulfonimidoyl)ethyl]-6-[(3R)-3-methylmorpholin-4-yl]pyrimidin-2-yl}-1H-benzimidazol-2-amine;
N-methyl-1-{4-[i-methyl-1-((R)—S-methylsulfonimidoyl)ethyl]-6-[(3R)-3-methylmorpholin-4-yl]pyrimidin-2-yl}-1H-benzimidazol-2-amine;
N-Methyl-1-{4-[(3R)-3-methylmorpholin-4-yl]-6-[4-((S)—S-methylsulfonimidoyl)tetrahydro-2H-pyran-4-yl]pyrimidin-2-yl}-1H-benzimidazol-2-amine;
N-methyl-1-{4-[(3R)-3-methylmorpholin-4-yl]-6-[4-((R)—S-methylsulfonimidoyl)tetrahydro-2H-pyran-4-yl]pyrimidin-2-yl}-1H-benzimidazol-2-amine;
4-{4-[(3R)-3-Methylmorpholin-4-yl]-6-[4-((S)—S-methylsulfonimidoyl)tetrahydro-2H-pyran-4-yl]pyrimidin-2-yl}-1H-indole;
4-Fluoro-N-methyl-1-{4-[1-methyl-1-((S)—S-methylsulfonimidoyl)ethyl]-6-[(3R)-3-methylmorpholin-4-yl]pyrimidin-2-yl}-1H-benzimidazol-2-amine;
4-fluoro-N-methyl-1-{4-[1-methyl-1-((R)—S-methylsulfonimidoyl)ethyl]-6-[(3R)-3-methylmorpholin-4-yl]pyrimidin-2-yl}-1H-benzimidazol-2-amine;
6-Fluoro-N-methyl-1-{4-[1-methyl-1-((R)—S-methylsulfonimidoyl)ethyl]-6-[(3R)-3-methylmorpholin-4-yl]pyrimidin-2-yl}-1H-benzimidazol-2-amine;
5-Fluoro-N-methyl-1-{4-[1-methyl-1-((R)—S-methylsulfonimidoyl)ethyl]-6-[(3R)-3-methylmorpholin-4-yl]pyrimidin-2-yl}-1H-benzimidazol-2-amine;
5-Fluoro-N-methyl-1-{4-[1-methyl-1-((S)—S-methylsulfonimidoyl)ethyl]-6-[(3R)-3-methylmorpholin-4-yl]pyrimidin-2-yl}-1H-benzimidazol-2-amine;
6-fluoro-N-methyl-1-{4-[1-methyl-1-((S)—S-methylsulfonimidoyl)ethyl]-6-[(3R)-3-methylmorpholin-4-yl]pyrimidin-2-yl}-1H-benzimidazol-2-amine;
6-Fluoro-N-methyl-1-{4-[(3R)-3-methylmorpholin-4-yl]-6-[1-((R)—S-methylsulfonimidoyl)cyclopropyl]pyrimidin-2-yl}-1H-benzimidazol-2-amine;
5-fluoro-N-methyl-1-{4-[(3R)-3-methylmorpholin-4-yl]-6-[1-((R)—S-methylsulfonimidoyl)cyclopropyl]pyrimidin-2-yl}-1H-benzimidazol-2-amine;
5-fluoro-N-methyl-1-{4-[(3R)-3-methylmorpholin-4-yl]-6-[1-((S)—S-methylsulfonimidoyl)cyclopropyl]pyrimidin-2-yl}-1H-benzimidazol-2-amine; and
6-fluoro-N-methyl-1-{4-[(3R)-3-methylmorpholin-4-yl]-6-[1-((S)—S-methylsulfonimidoyl)cyclopropyl]pyrimidin-2-yl}-1H-benzimidazol-2-amine,
or a pharmaceutically acceptable salt thereof.

In another aspect, the small molecule ATR inhibitor is a compound selected from any one of
4-{4-[(3R)-3-methylmorpholin-4-yl]-6-[(R)—(S-methylsulfonimidoyl)methyl]pyrimidin-2-yl}-1H-pyrrolo[2,3-b]pyridine;
4-{4-[(3R)-3-methylmorpholin-4-yl]-6-[1-((S)—S-methylsulfonimidoyl)cyclopropyl]pyrimidin-2-yl}-1H-pyrrolo[2,3-b]pyridine;
4-{4-[(3R)-3-methylmorpholin-4-yl]-6-[1-((R)—S-methylsulfonimidoyl)cyclopropyl]pyrimidin-2-yl}-1H-pyrrolo[2,3-b]pyridine;
N-methyl-1-{4-[(3R)-3-methylmorpholin-4-yl]-6-[1-(R)—(S-methylsulfonimidoyl)cyclopropyl]pyrimidin-2-yl}-1H-benzimidazol-2-amine; and
N-methyl-1-{4-[(3R)-3-methylmorpholin-4-yl]-6-[1-(S)—(S-methylsulfonimidoyl)cyclopropyl]pyrimidin-2-yl}-1H-benzimidazol-2-amine,
or a pharmaceutically acceptable salt thereof.

In some embodiments, the small molecule ATR inhibitor is a compound of Formula (VI) (ceralasertib):

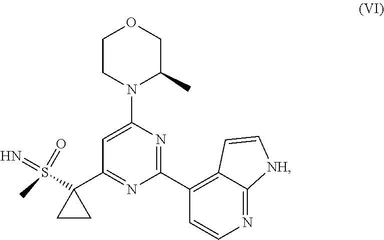 [SMILES: C[C@@H]1COCCN1c1cc(C2([S@](C)(=N)=O)CC2)nc(-c2ccnc3[nH]ccc23)n1]

which is 4-{4-[(3R)-3-methylmorpholin-4-yl]-6-[1-((R)—S-methylsulfonimidoyl)cyclopropyl]pyrimidin-2-yl}-1H-pyrrolo[2,3-b]pyridine, or a pharmaceutically acceptable salt thereof.

Various combinations of KRAS inhibitors and ATR inhibitors are envisioned by the present disclosure. For example, in some embodiments, the combinations of KRAS inhibitors and ATR inhibitors can include one of the following combinations as found in Table 4:

TABLE 4

| Combo | KRAS | ATR |
|---|---|---|
| # | inhibitor | inhibitor |
| 1 | sotorasib | berzosertib |
| 2 | sotorasib | elimusertib |
| 3 | sotorasib | camonsertib |
| 4 | sotorasib | gartisertib |
| 5 | sotorasib | M1774 |
| 6 | sotorasib | compound of |
| | | Formula (V) |
| 7 | sotorasib | ceralasertib |
| 8 | sotorasib | ATG-018 |
| 9 | sotorasib | ART0380 |
| 10 | sotorasib | IMP9064 |
| 11 | adagrasib | berzosertib |
| 12 | adagrasib | elimusertib |
| 13 | adagrasib | camonsertib |
| 14 | adagrasib | gartisertib |
| 15 | adagrasib | M1774 |
| 16 | adagrasib | compound of |
| | | Formula (V) |
| 17 | adagrasib | ceralasertib |
| 18 | adagrasib | ATG-018 |
| 19 | adagrasib | ART0380 |
| 20 | adagrasib | IMP9064 |
| 21 | compound of | berzosertib |
| | Formula (I) | |
| 22 | compound of | elimusertib |
| | Formula (I) | |
| 23 | compound of | camonsertib |
| | Formula (I) | |
| 24 | compound of | gartisertib |
| | Formula (I) | |
| 25 | compound of | M1774 |
| | Formula (I) | |
| 26 | compound of | compound of |
| | Formula (I) | Formula (V) |
| 27 | compound of | ceralasertib |
| | Formula (I) | |
| 28 | compound of | ATG-018 |
| | Formula (I) | |
| 29 | compound of | ART0380 |
| | Formula (I) | |
| 30 | compound of | IMP9064 |
| | Formula (I) | |
| 31 | compound of | berzosertib |
| | Formula (II) | |
| 32 | compound of | elimusertib |
| | Formula (II) | |
| 33 | compound of | camonsertib |
| | Formula (II) | |
| 34 | compound of | gartisertib |
| | Formula (II) | |
| 35 | compound of | M1774 |
| | Formula (II) | |
| 36 | compound of | compound of |
| | Formula (II) | Formula (V) |
| 37 | compound of | ceralasertib |
| | Formula (II) | |
| 38 | compound of | ATG-018 |
| | Formula (II) | |
| 39 | compound of | ART0380 |
| | Formula (II) | |
| 40 | compound of | IMP9064 |
| | Formula (II) | |
| 41 | compound of | berzosertib |
| | Formula (III) | |
| 42 | compound of | elimusertib |
| | Formula (III) | |
| 43 | compound of | camonsertib |
| | Formula (III) | |
| 44 | compound of | gartisertib |
| | Formula (III) | |
| 45 | compound of | M1774 |
| | Formula (III) | |
| 45 | compound of | compound of |
| | Formula (III) | Formula (V) |
| 46 | compound of | ceralasertib |
| | Formula (III) | |
| 47 | compound of | ATG-018 |
| | Formula (III) | |
| 49 | compound of | ART0380 |
| | Formula (III) | |
| 50 | compound of | IMP9064 |
| | Formula (III) | |
| 51 | compound of | berzosertib |
| | Formula (IV) | |
| 52 | compound of | elimusertib |
| | Formula (IV) | |
| 53 | compound of | camonsertib |
| | Formula (IV) | |
| 54 | compound of | gartisertib |
| | Formula (IV) | |
| 55 | compound of | M1774 |
| | Formula (IV) | |
| 56 | compound of | compound of |
| | Formula (IV) | Formula (V) |
| 57 | compound of | ceralasertib |
| | Formula (IV) | |
| 58 | compound of | ATG-018 |
| | Formula (IV) | |
| 59 | compound of | ART0380 |
| | Formula (IV) | |
| 60 | compound of | IMP9064 |
| | Formula (IV) | |
| 61 | D35-001 | berzosertib |
| 62 | D35-001 | elimusertib |
| 63 | D35-001 | camonsertib |
| 64 | D35-001 | gartisertib |
| 65 | D35-001 | M1774 |
| 66 | D35-001 | compound of |
| | | Formula (V) |
| 67 | D35-001 | ceralasertib |
| 68 | D35-001 | ATG-018 |
| 68 | D35-001 | ART0380 |
| 70 | D35-001 | IMP9064 |
| 71 | JAB-21822 | berzosertib |
| 72 | JAB-21822 | elimusertib |
| 73 | JAB-21822 | camonsertib |
| 74 | JAB-21822 | gartisertib |
| 75 | JAB-21822 | M1774 |
| 76 | JAB-21822 | compound of |
| | | Formula (V) |
| 77 | JAB-21822 | ceralasertib |
| 78 | JAB-21822 | ATG-018 |
| 79 | JAB-21822 | ART0380 |
| 80 | JAB-21822 | IMP9064 |
| 81 | HBI-2438 | berzosertib |
| 82 | HBI-2438 | elimusertib |
| 83 | HBI-2438 | camonsertib |
| 84 | HBI-2438 | gartisertib |
| 85 | HBI-2438 | M1774 |
| 86 | HBI-2438 | compound of |
| | | Formula (V) |
| 87 | HBI-2438 | ceralasertib |
| 88 | HBI-2438 | ATG-018 |
| 89 | HBI-2438 | ART0380 |
| 90 | HBI-2438 | IMP9064 |
| 91 | YL-15293 | berzosertib |
| 92 | YL-15293 | elimusertib |
| 93 | YL-15293 | camonsertib |
| 94 | YL-15293 | gartisertib |
| 95 | YL-15293 | M1774 |
| 96 | YL-15293 | compound of |
| | | Formula (V) |
| 97 | YL-15293 | ceralasertib |
| 98 | YL-15293 | ATG-018 |
| 99 | YL-15293 | ART0380 |
| 100 | YL-15293 | IMP9064 |
| 101 | GEC255 | berzosertib |
| 102 | GEC255 | elimusertib |
| 103 | GEC255 | camonsertib |
| 104 | GEC255 | gartisertib |
| 105 | GEC255 | M1774 |
| 106 | GEC255 | compound of |
| | | Formula (V) |
| 107 | GEC255 | ceralasertib |
| 108 | GEC255 | ATG-018 |
| 109 | GEC255 | ART0380 |
| 110 | GEC255 | IMP9064 |
| 111 | IBI351 | berzosertib |
| 112 | IBI351 | elimusertib |
| 113 | IBI351 | camonsertib |
| 114 | IBI351 | gartisertib |
| 115 | IBI351 | M1774 |
| 116 | IBI351 | compound of |
| | | Formula (V) |
| 117 | IBI351 | ceralasertib |
| 118 | IBI351 | ATG-018 |
| 119 | IBI351 | ART0380 |
| 120 | IBI351 | IMP9064 |
| 121 | RMC-6291 | berzosertib |
| 122 | RMC-6291 | elimusertib |
| 123 | RMC-6291 | camonsertib |
| 124 | RMC-6291 | gartisertib |
| 125 | RMC-6291 | M1774 |
| 126 | RMC-6291 | compound of |
| | | Formula (V) |
| 127 | RMC-6291 | ceralasertib |
| 128 | RMC-6291 | ATG-018 |
| 129 | RMC-6291 | ART0380 |
| 130 | RMC-6291 | IMP9064 |
| 131 | MK-1084 | berzosertib |
| 132 | MK-1084 | elimusertib |
| 133 | MK-1084 | camonsertib |
| 134 | MK-1084 | gartisertib |
| 135 | MK-1084 | M1774 |
| 136 | MK-1084 | compound of |
| | | Formula (V) |
| 137 | MK-1084 | ceralasertib |
| 138 | MK-1084 | ATG-018 |
| 139 | MK-1084 | ART0380 |
| 140 | MK-1084 | IMP9064 |
| 141 | MRTX1133 | berzosertib |
| 142 | MRTX1133 | elimusertib |
| 143 | MRTX1133 | camonsertib |
| 144 | MRTX1133 | gartisertib |
| 145 | MRTX1133 | M1774 |
| 146 | MRTX1133 | compound of |
| | | Formula (V) |
| 147 | MRTX1133 | ceralasertib |
| 148 | MRTX1133 | ATG-018 |
| 149 | MRTX1133 | ART0380 |
| 150 | MRTX1133 | IMP9064 |
| 151 | HRS-4642 | berzosertib |
| 152 | HRS-4642 | elimusertib |
| 153 | HRS-4642 | camonsertib |
| 154 | HRS-4642 | gartisertib |
| 155 | HRS-4642 | M1774 |
| 156 | HRS-4642 | compound of |
| | | Formula (V) |
| 157 | HRS-4642 | ceralasertib |
| 158 | HRS-4642 | ATG-018 |
| 159 | HRS-4642 | ART0380 |
| 160 | HRS-4642 | IMP9064 |
| 161 | RMC-9805 | berzosertib |
| 163 | RMC-9805 | elimusertib |
| 163 | RMC-9805 | camonsertib |
| 164 | RMC-9805 | gartisertib |
| 165 | RMC-9805 | M1774 |
| 166 | RMC-9805 | compound of |
| | | Formula (V) |
| 167 | RMC-9805 | ceralasertib |
| 168 | RMC-9805 | ATG-018 |
| 169 | RMC-9805 | ART0380 |
| 170 | RMC-9805 | IMP9064 |
| 171 | Ly3537982 | berzosertib |
| 172 | Ly3537982 | elimusertib |
| 173 | Ly3537982 | camonsertib |
| 174 | Ly3537982 | gartisertib |
| 175 | Ly3537982 | M1774 |
| 176 | Ly3537982 | compound of |
| | | Formula (V) |
| 177 | Ly3537982 | ceralasertib |
| 178 | Ly3537982 | ATG-018 |
| 179 | Ly3537982 | ART0380 |
| 180 | Ly3537982 | IMP9064 |
| 181 | GDC-6036 | berzosertib |
| 182 | GDC-6036 | elimusertib |
| 183 | GDC-6036 | camonsertib |
| 184 | GDC-6036 | gartisertib |
| 185 | GDC-6036 | M1774 |
| 186 | GDC-6036 | compound of |
| | | Formula (V) |
| 187 | GDC-6036 | ceralasertib |
| 188 | GDC-6036 | ATG-018 |
| 189 | GDC-6036 | ART0380 |
| 190 | GDC-6036 | IMP9064 |
| 191 | D-1553 | berzosertib |
| 192 | D-1553 | elimusertib |
| 193 | D-1553 | camonsertib |
| 194 | D-1553 | gartisertib |
| 195 | D-1553 | M1774 |
| 196 | D-1553 | compound of |
| | | Formula (V) |
| 197 | D-1553 | ceralasertib |
| 198 | D-1553 | ATG-018 |
| 199 | D-1553 | ART0380 |
| 200 | D-1553 | IMP9064 |
| 201 | JDQ443 | berzosertib |
| 202 | JDQ443 | elimusertib |
| 203 | JDQ443 | camonsertib |
| 204 | JDQ443 | gartisertib |
| 205 | JDQ443 | M1774 |
| 206 | JDQ443 | compound of |
| | | Formula (V) |
| 207 | JDQ443 | ceralasertib |
| 208 | JDQ443 | ATG-018 |
| 209 | JDQ443 | ART0380 |
| 210 | JDQ443 | IMP9064 |
| 211 | BI1823911 | berzosertib |
| 212 | BI1823911 | elimusertib |
| 213 | BI1823911 | camonsertib |
| 214 | BI1823911 | gartisertib |
| 215 | BI1823911 | M1774 |
| 216 | BI1823911 | compound of |
| | | Formula (V) |
| 217 | BI1823911 | ceralasertib |
| 218 | BI1823911 | ATG-018 |
| 219 | BI1823911 | ART0380 |
| 220 | BI1823911 | IMP9064 |
| 221 | GFH925 | berzosertib |
| 222 | GFH925 | elimusertib |
| 223 | GFH925 | camonsertib |
| 224 | GFH925 | gartisertib |
| 225 | GFH925 | M1774 |
| 226 | GFH925 | compound of |
| | | Formula (V) |
| 227 | GFH925 | ceralasertib |
| 228 | GFH925 | ATG-018 |
| 229 | GFH925 | ART0380 |
| 230 | GFH925 | IMP9064 |
| 231 | JAB-2200 | berzosertib |
| 232 | JAB-2200 | elimusertib |
| 233 | JAB-2200 | camonsertib |
| 234 | JAB-2200 | gartisertib |
| 235 | JAB-2200 | M1774 |
| 236 | JAB-2200 | compound of |
| | | Formula (V) |
| 237 | JAB-2200 | ceralasertib |
| 238 | JAB-2200 | ATG-018 |
| 239 | JAB-2200 | ART0380 |
| 240 | JAB-2200 | IMP9064 |
| 241 | Vrtx153 | berzosertib |
| 242 | Vrtx153 | elimusertib |
| 243 | Vrtx153 | camonsertib |
| 244 | Vrtx153 | gartisertib |
| 245 | Vrtx153 | M1774 |
| 246 | Vrtx153 | compound of |
| | | Formula (V) |
| 247 | Vrtx153 | ceralasertib |
| 248 | Vrtx153 | ATG-018 |
| 249 | Vrtx153 | ART0380 |
| 250 | Vrtx153 | IMP9064 |
| 252 | ERAS-4 | berzosertib |
| 252 | ERAS-4 | elimusertib |
| 253 | ERAS-4 | camonsertib |
| 254 | ERAS-4 | gartisertib |
| 255 | ERAS-4 | M1774 |
| 256 | ERAS-4 | compound of |
| | | Formula (V) |
| 257 | ERAS-4 | ceralasertib |
| 258 | ERAS-4 | ATG-018 |
| 259 | ERAS-4 | ART0380 |
| 260 | ERAS-4 | IMP9064 |
| 261 | ASP3082 | berzosertib |
| 262 | ASP3082 | elimusertib |
| 263 | ASP3082 | camonsertib |
| 264 | ASP3082 | gartisertib |
| 265 | ASP3082 | M1774 |
| 266 | ASP3082 | compound of |
| | | Formula (V) |
| 267 | ASP3082 | ceralasertib |
| 268 | ASP3082 | ATG-018 |
| 269 | ASP3082 | ART0380 |
| 270 | ASP3082 | IMP9064 |
| 271 | RMC-0708 | berzosertib |
| 272 | RMC-0708 | elimusertib |
| 273 | RMC-0708 | camonsertib |
| 274 | RMC-0708 | gartisertib |
| 275 | RMC-0708 | M1774 |
| 276 | RMC-0708 | compound of |
| | | Formula (V) |
| 277 | RMC-0708 | ceralasertib |
| 278 | RMC-0708 | ATG-018 |
| 279 | RMC-0708 | ART0380 |
| 280 | RMC-0708 | IMP9064 |
| 281 | RMC-8839 | berzosertib |
| 282 | RMC-8839 | elimusertib |
| 283 | RMC-8839 | camonsertib |
| 284 | RMC-8839 | gartisertib |
| 285 | RMC-8839 | M1774 |
| 286 | RMC-8839 | compound of |
| | | Formula (V) |
| 287 | RMC-8839 | ceralasertib |
| 288 | RMC-8839 | ATG-018 |
| 289 | RMC-8839 | ART0380 |
| 290 | RMC-8839 | IMP9064 |
| 291 | RMC-6291 | berzosertib |
| 292 | RMC-6291 | elimusertib |
| 293 | RMC-6291 | camonsertib |
| 294 | RMC-6291 | gartisertib |
| 295 | RMC-6291 | M1774 |
| 296 | RMC-6291 | compound of |
| | | Formula (V) |
| 297 | RMC-6291 | ceralasertib |
| 298 | RMC-6291 | ATG-018 |
| 299 | RMC-6291 | ART0380 |
| 300 | RMC-6291 | IMP9064 |
| 301 | JAB-23400 | berzosertib |
| 302 | JAB-23400 | elimusertib |
| 303 | JAB-23400 | camonsertib |
| 304 | JAB-23400 | gartisertib |
| 305 | JAB-23400 | M1774 |
| 306 | JAB-23400 | compound of |
| | | Formula (V) |
| 307 | JAB-23400 | ceralasertib |
| 308 | JAB-23400 | ATG-018 |
| 309 | JAB-23400 | ART0380 |
| 310 | JAB-23400 | IMP9064 |
| 311 | TEB-17231 | berzosertib |
| 312 | TEB-17231 | elimusertib |
| 313 | TEB-17231 | camonsertib |
| 314 | TEB-17231 | gartisertib |
| 315 | TEB-17231 | M1774 |
| 316 | TEB-17231 | compound of |
| | | Formula (V) |
| 317 | TEB-17231 | ceralasertib |
| 318 | TEB-17231 | ATG-018 |
| 319 | TEB-17231 | ART0380 |
| 320 | TEB-17231 | IMP9064 |
| 321 | QTX3046 | berzosertib |
| 322 | QTX3046 | elimusertib |
| 323 | QTX3046 | camonsertib |
| 324 | QTX3046 | gartisertib |
| 325 | QTX3046 | M1774 |
| 326 | QTX3046 | compound of |
| | | Formula (V) |
| 327 | QTX3046 | ceralasertib |
| 328 | QTX3046 | ATG-018 |
| 329 | QTX3046 | ART0380 |
| 330 | QTX3046 | IMP9064 |

In some embodiments, the (i) KRAS inhibitor, and (ii) ATR inhibitor is one of the combinations 1-10, 11-20, 21-30, 31-40, 41-50, 51-60, 61-70, 71-80, 81-90, 91-100, 101-110, 111-120, 121-130, 131-140, 141-150, 151-160, 161-170, 171-180, 181-190, 191-200, 201-210, 211-220, 221-230, 231-240, 241-250, 251-260, 261-270, 271-280, 281-290, 291-300, 301-310, 311-320, or 321-330. In some embodiments, the (i) KRAS inhibitor is a compound of Formula (I), and (ii) ATR inhibitor is ceralasertib. In some embodiments, the (i) KRAS inhibitor is a compound of Formula (II), and (ii) ATR inhibitor is ceralasertib. In some embodiments, the (i) KRAS inhibitor is a compound of Formula (III), and (ii) ATR inhibitor is ceralasertib. In some embodiments, the (i) KRAS inhibitor is a compound of Formula (IV), and (ii) ATR inhibitor is ceralasertib.

In some embodiments, the subject has a KRAS, NRAS or HRAS mutation. In some embodiments, the subject has a KRAS mutation. In some embodiments, the KRAS inhibitors described herein, e.g., the antibodies or small molecule KRAS inhibitors, selectively target mutations at codon 12, codon 13 and/or codon 61. In some embodiments, the mutations in codon 12 or codon 13 results in downregulation through inducing intermolecular beta-sheet formation, but do not substantially alter the amount or biological activity of wild-type RAS. From this perspective, the term “wild-type” may be ascribed to the conventional meaning of the KRAS variant encoded by the allele of the respective KRAS gene that is most commonly observed in a human population.

In some embodiments, the subject has a “G12 mutant human KRAS” wherein the glycine residue at position 12 (G12) has been mutated. In some embodiments, the subject has a KRAS protein in which G12 has been deleted, i.e., a deletion mutant. In some embodiments, the subject has a KRAS protein in which an additional amino acid is encoded proximately upstream or downstream from G12, i.e., an insertion mutant. In some embodiments, the subject has a KRAS protein in which G12 has been replaced by exactly one amino acid other than glycine (G12 missense mutant KRAS). Missense mutations replacing G12 of human RAS with virtually every other amino acid have been documented in diseases, including G12A, G12D, G12F, G12L, G12P, G12S, G12V, G12Y, G12C, G12E, G121, G12N, G12R, G12T, and G12W missense mutations. G12Q, G12H, G12K, and G12M missense mutations are also conceivable.

In some embodiments, a “G13 mutant human KRAS” has glycine residue at position 13 (G13) mutated. Particularly intended are mutant KRAS proteins in which G13 has been replaced by exactly one amino acid other than glycine (G13 missense mutant KRAS). Missense mutations replacing G13 of human RAS with virtually every other amino acid have been documented in diseases, including G13A, G13D, G13F, G13M, G13P, G13S, G13Y, G13C, G13E, G131, G13N, G13R, and G13V missense mutations. G13L, G13W, G13H, G13K, G13Q and G13T missense mutations are also conceivable.

In some embodiments, a “Q61 mutant human KRAS” has glutamine residue at position 61 (Q61) mutated. Particularly intended are mutant KRAS proteins in which Q61 has been replaced by exactly one amino acid other than glutamine (Q61 missense mutant KRAS). Missense mutations replacing Q61 of human KRAS can include Q61A, Q61D, Q61F, Q61M, Q61P, Q61S, Q61Y, Q61C, Q61E, Q611, Q61N, Q61R, and Q61V missense mutations. Q61L, Q61W, Q61H, Q61K, Q61G and Q61T missense mutations are also conceivable.

In some embodiments, G12, G13 or Q61 mutant human KRAS proteins, in particular G12 or G13 missense mutants, may cause or be associated with a proliferative or neoplastic disease and/or may result in a constitutively active KRAS, more particularly KRAS that is defective in GAP-mediated GTP hydrolysis.

In some embodiments, the methods described herein can be used when amino acid at position 12, 13, or 61 is replaced from wild-type by an uncharged amino acid. In some embodiments, the mutant at amino acid at position 12, 13, or 61 of human KRAS protein can be one in which amino acid at position 12, 13, or 61 is replaced by a hydrophobic amino acid other than proline, such as glycine (G), alanine (A), valine (V), leucine (L), isoleucine (I), phenylalanine (F), methionine (M), and tryptophan (W). In some embodiments, the mutant amino acid at position 12, 13, or 61 of human KRAS protein can be one in which the amino acid at position 12, 13, or 61 is replaced by a polar amino acid, such as serine (S), threonine (T), cysteine (C), asparagine (N), glutamine (Q), or tyrosine (Y). In some embodiments, the mutant amino acid at position 12, or 13 of human KRAS protein may be a G12V, G12C, G12A, or G12S mutant human KRAS protein, such as a G12V, G12C, G12A, or G12S mutant human KRAS, NRAS or HRAS protein, e.g., a G12V, G12C, G12A, or G12S mutant human KRAS protein. In particularly preferred embodiments, the G12 mutant human KRAS protein may be a G12V mutant human KRAS protein, such as a G12V mutant human KRAS protein, e.g., a G12V mutant human KRAS protein. In some embodiments, the G13 mutant human KRAS protein may be a G13V, G13C, or G13S mutant human KRAS protein. In some embodiments, the KRAS mutation is a mutation at codon 12, codon 13 and/or codon 61. In some embodiments, the KRAS mutation is a mutation at codon 12. In some embodiments, the KRAS mutation is a G12C mutation.

In some embodiments, the mutation at codon 12, codon 13 and/or codon 61 can result in proliferative or neoplastic disease in a subject, e.g., a human subject. In some embodiments, the subject has lung cancer, colorectal cancer, pancreatic cancer or combination thereof. In some embodiments, the subject has mutations at codon 12, codon 13 and/or codon 61 and has been diagnosed with lung cancer, colorectal cancer, pancreatic cancer or combination thereof. In some embodiments, the subject has lung, colorectal, pancreatic, esophagogastric cancer, endometrial cancer, cholangiocarcinoma or combination thereof. In some embodiments, the subject is a human.

In some embodiments, the methods described herein can be administered for modulating the adaptive immune response. In some embodiments, modulating the adaptive immune response provides deep and durable anti-tumor activity wherein the subject develops a long-term immunogenic memory response against the tumor. In some embodiments, tumor growth after cessation of administration of the KRAS inhibitor, and the ATR inhibitor continues to be diminished and/or eliminated and/or reduced. Thus, in some embodiments, the methods and compositions described in the present disclosure provide a method of modulating the adaptive immune response in a subject, the method comprising administering a composition comprising: (a) a KRAS inhibitor, and (b) an ATR inhibitor.

In some embodiments, the methods and compositions described in the present disclosure can reduce the size of a tumor in a subject. In some embodiments, the methods and compositions described in the present disclosure are directed to reducing the volume of a cancerous tumor in a subject, the method comprising administering a composition comprising: (a) a KRAS inhibitor, and (b) an ATR inhibitor.

In some embodiments, the methods and compositions described in the present disclosure can treat a subject in need thereof, wherein the method comprises administering a composition comprising (a) a KRAS inhibitor, and (b) an ATR inhibitor, wherein the subject has a disorder mediated by a KRAS, NRAS or HRAS G12C mutation.

In some embodiments, the disclosure provides a composition comprising: (a) a KRAS inhibitor, and (b) an ATR inhibitor. In some embodiments, the composition is an oral dosage form, e.g., liquids, capsules, tablets, or chewable tablets, an injectable dosage form (e.g., intramuscular or intravenous dosage form, a sublingual or transmucosal dosage form, a nasal dosage form, an inhalable dosage form, or cutaneous dosage form, or a transdermal dosage form. One of skill in the art can include appropriate excipients, diluents, binders, etc., in accordance with the mode of administration and add such to the compositions described herein. In some embodiments, the composition comprising (a) a KRAS inhibitor, and (b) an ATR inhibitor is pharmaceutically acceptable. “Pharmaceutically acceptable” as used herein to describe the compositions comprising the KRAS inhibitor/ATR inhibitor means approved by a regulatory agency of a Federal or state government, or listed in the U.S. Pharmacopeia, European Pharmacopia or other generally recognized pharmacopeia for use in animals, and more particularly in humans. In some embodiments, the term “pharmaceutically acceptable” refers to those compositions and/or dosage forms comprising the KRAS inhibitor/ATR inhibitor which are, within the scope of sound medical judgment, suitable for use in contact with the tissues of human beings and animals without excessive toxicity, irritation, allergic response, or other problem or complication, commensurate with a reasonable benefit/risk ratio.

Various combinations of (a) a KRAS inhibitor, and (b) an ATR inhibitor can be used in the present disclosure. In some embodiments, the KRAS inhibitor is sotorasib, adagrasib, Ly3537982, GDC-6036, D-1553, JDQ443, BI1823911, GFH925, D35-001, JAB-21822, HBI-2438, YL-15293, GEC255, IBI351, RMC-6291, MK-1084, MRTX1133, HRS-4642, RMC-9805, JAB-2200, Vrtx153, ERAS-4, ASP3082, RMC-0708, RMC 8839, RMC-6291, JAB-23400, TEB-17231, QTX3046, a compound of Formula (I), a compound of Formula (II), a compound of Formula (III), or a compound of Formula (IV). In some embodiments, the ATR inhibitor is M6620/VX970 (berzosertib), BAY-1895344 (elimusertib), RP-3500 (camonsertib), ATRN-119, SC0245, ATRN-212, LR-02, M4344 (Gartisertib), M1774, ATG-018, ARTO380, BG-129, JS-123, BKT-300, AZ-20, VE-821, AZD-559 or IMP9064, a compound of Formula (V), or a compound of Formula (VI).

In some embodiments, the composition comprises a combination of (i) KRAS inhibitor, and (ii) ATR inhibitor as found in Table 4. In some embodiments, the composition comprises a combination of (i) KRAS inhibitor, and (ii) ATR inhibitor as found in combinations 1-10, 11-20, 21-30, 31-40, 41-50, 51-60, 61-70, 71-80, 81-90, 91-100, 101-110, 111-120, 121-130, 131-140, 141-150, 151-160, 161-170, 171-180, 181-190, 191-200, 201-210, 211-220, 221-230, 231-240, 241-250, 251-260, 261-270, 271-280, 281-290, 291-300, 301-310, 311-320, or 321-330 as described in Table 4. In some embodiments, the composition comprises (i) a KRAS inhibitor compound of Formula (I), and (ii) ceralasertib. In some embodiments, the composition comprises (i) a KRAS inhibitor of Formula (II), and (ii) ceralasertib. In some embodiments, the composition comprises (i) a KRAS inhibitor of Formula (III), and (ii) ceralasertib. In some embodiments, the composition comprises (i) a KRAS inhibitor of Formula (IV), and (ii) ceralasertib.

In some embodiments, the composition comprising the KRAS inhibitor and ATR inhibitor can be in a physical state suitable for appropriate modes of administration. In some embodiments, the composition is a liquid. In some embodiments, the composition is a solid. In some embodiments, the composition is a solid oral dosage form. In some embodiments, the composition further comprises a pharmaceutically acceptable excipient, diluent or carrier.

In some embodiments, the disclosure provides a use of the compositions as described herein, for the manufacture of a medicament, for example a medicament for the treatment of cancer.

The present disclosure describes how the administration of both a KRAS inhibitor and an ATR inhibitor can be used to treat a cancer. In some embodiments, both the KRAS inhibitor and the ATR inhibitor are in the same composition. However, in some embodiments, the KRAS inhibitor and the ATR inhibitor are in separate compositions but are in a kit such that both the KRAS inhibitor and the ATR inhibitor can be administered to the same subject to treat a condition, e.g., a cancer. In some embodiments, the kit comprises (a) a first container comprising a first composition comprising a KRAS inhibitor, and (b) a second container comprising a second composition comprising an ATR inhibitor. In some embodiments, the kit further comprises instructions for administering the KRAS inhibitor and ATR inhibitor, include modes of administration, dosing amounts, and dosing regimens.

In some embodiments, the KRAS inhibitor in the kit is sotorasib, adagrasib, Ly3537982, GDC-6036, D-1553, JDQ443, BI1823911, GFH925, D35-001, JAB-21822, HBI-2438, YL-15293, GEC255, IBI351, RMC-6291, MK-1084, MRTX1133, HRS-4642, RMC-9805, JAB-2200, Vrtx153, ERAS-4, ASP3082, RMC-0708, RMC-8839, RMC-6291, JAB-23400, TEB-17231, QTX3046, a compound of Formula (I), a compound of Formula (II), a compound of Formula (III), or a compound of Formula (IV). In some embodiments, the ATR inhibitor in the kit is M6620/VX970 (berzosertib), BAY-1895344 (elimusertib), RP-3500 (camonsertib), ATRN-119, SC0245, ATRN-212, LR-02, M4344 (Gartisertib), M1774, ATG-018, ARTO380, BG-129, JS-123, BKT-300, AZ-20, VE-821, AZD-559 or IMP9064, a compound of Formula (V), or a compound of Formula (VI).

In some embodiments, the dosage forms in the kits can be the same, e.g., both the KRAS inhibitor and ATR inhibitor are both oral dosage forms. In some embodiments, the dosage forms in the kit can be different, e.g., the KRAS inhibitor is an oral dosage form, and the ATR inhibitor is in an intravenous dosage form. In some embodiments, the first composition of the kit, the second composition of the kit, or both the first composition and the second composition of the kit is/are a liquid. In some embodiments, the first composition of the kit, the second composition of the kit, or both the first composition and the second composition of the kit is/are a solid. In some embodiments, the first composition of the kit, the second composition of the kit, or both the first composition and the second composition of the kit is/are a solid oral dosage form. In some embodiments, the first composition of the kit, the second composition of the kit, or both the first composition and the second composition of the kit further comprise a pharmaceutically acceptable excipient, diluent or carrier.

All references cited herein, including patents, patent applications, papers, textbooks and the like, and the references cited therein, to the extent that they are not already, are hereby incorporated herein by reference in their entirety.

### EXAMPLES

All pathogen-free animals used in the studies below were housed in a controlled, pathogen-free environment with the following conditions: temperature (19-23° C.), humidity (55% 10%), photoperiod (12 h light/dark cycle), and air exchange. Animals were housed in individually vented cages, with food and water provided ad libitum.

Following testing materials were used for the preclinical, in vivo studies below: vehicle (0.5% HPMC/0.1% Tween 80), AZD4625 (0.5% HPMC/0.1% Tween 80), Compound of Formula IV (0.5% HPMC/0.1% Tween 80) and AZD6738 (10% DMSO/40% PG/50% WFI). For the monotherapies, the vehicle and the inhibitors were dosed orally in a volume of 0.05-0.1 mL/10 g. For the combination therapies, AZD4625 or Compound of Formula IV were dosed 4 or 2 hours after the AM dose of AZD6738.

Tumors were measured three times a week by caliper and the volumes were calculated using elliptical formula (pi/6×width×width×length). Animal bodyweight and tumor condition were also recorded for the duration of the study. Subject mice remained on study until tumor size reached 1.5 cm³ for anti-tumor studies and 0.7 cm³ for the rechallenge study, clinical signs were observed, or until the study end date.

### Example 1

The anti-tumor activity through dual inhibition of KRAS and ATR in KRAS mutant cancers was determined in a preclinical, in vivo study using KRAS^{G12C} and ATR inhibitors. The KRAS^{G12C} and ATR inhibitors used were AZD4625 (covalent allosteric inhibitor of KRAS^{G12C}) and AZD6738 (ceralasertib, an ATP-competitive ATR inhibitor).

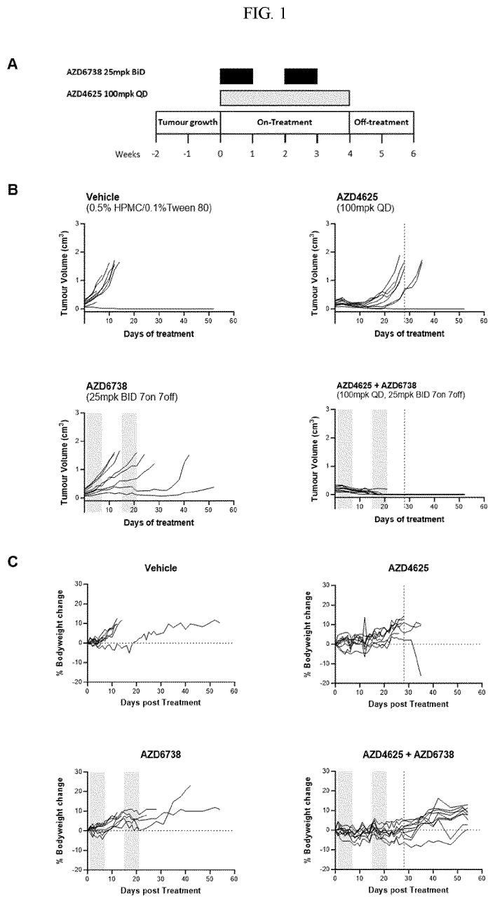

For the study, CT26^{G12C} murine colon carcinoma cells, engineered to express the KRAS^{G12C} mutation, were grown subcutaneously in BALB/c mice with an intact immune system. To prepare the subject mice, 5×10⁵ and 5×10⁴ CT26^{G12C} (G12C clone P5E6) cells were implanted subcutaneously into the right flank of female BALB/c mice (Envigo UK, Strain ID: HSD-162) or female nude-Foxn1nu mice (Envigo UK, Strain ID: HSD-069), respectively. Once a mean volume of approximately 0.2 cm³ tumor formed, subject mice were randomized into relevant treatment and control groups and treated with vehicle control, AZD4625 or AZD6738 in monotherapy, or in combination therapy for 4 weeks. AZD4625 was dosed daily and AZD6738 was dosed twice daily on a 7 day-on, 7 day-off schedules for the treatment period (FIG. 1A). Tumor growth and bodyweights of animals were monitored during and post-treatment. Subject mice remained on study until tumor size reached 1.5 cm³, clinical signs were observed, or to the study end date (day 54).

Result: Treatment groups treated with AZD4625 or AZD6738 monotherapy initially showed delayed tumor growth and extended survival rate compared to the control group, but most tumors ultimately returned (FIG. 1B, “Vehicle,” “AZD4625” and “AZD6738”). In contrast, the treatment group treated with AZD4625 and AZD6738 combination therapy showed complete tumor regression with responses being durable off drug, with 9 out of 10 animals maintaining complete tumor regression for the study period even after drug cessation (FIG. 1Bm “AZD4625+AZD6738”). All treatments were well tolerated with minimal impact on body weights (Figure iC).

### Example 2

Anti-tumor activity through dual inhibition of KRAS and ATR in KRAS mutant cancers was determined in a second, in vivo study using KRAS^{G12C} and ATR inhibitors. The KRAS^{G12C} and ATR inhibitors used were AZD4625 (covalent allosteric inhibitor of KRAS^{G12C}) Compound of Formula IV (a second, chemically different allosteric covalent KRAS^{G12C} inhibitor), and AZD6738 (ceralasertib, an ATP-competitive ATR inhibitor).

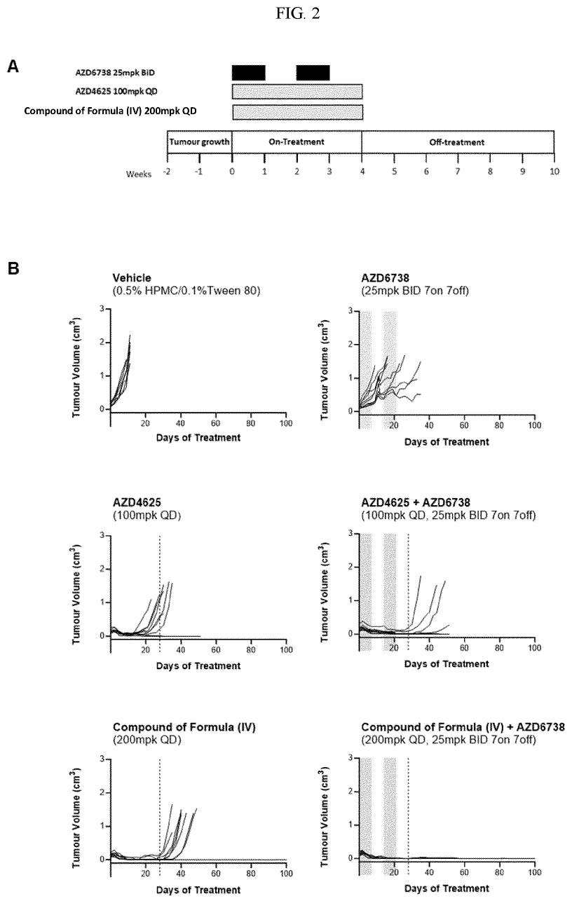

For the study, CT26^{G12C} murine colon carcinoma cells, engineered to express the KRAS^{G12C} mutation, were grown subcutaneously in BALB/c mice with an intact immune system. To prepare the subject mice, 5×10⁵ and 5×10⁴ CT26^{G12C} (G12C clone P5E6) cells were implanted subcutaneously into the right flank of female BALB/c mice (Envigo UK, Strain ID: HSD-162) or female nude-Foxn1nu mice (Envigo UK, Strain ID: HSD-069), respectively. Once a mean volume of approximately 0.2 cm³ tumor formed, subject mice were randomized into relevant treatment and control groups and treated with vehicle control, AZD4625, Compound of Formula IV, or AZD6738 in monotherapy, or AZD4625 and AZD6738 or Compound of Formula IV and AZD6738 in combination therapy for 4 weeks. AZD4625 and Compound of Formula IV were dosed daily and AZD6738 was dosed twice daily on a 7 day-on, 7 day-off schedules for the treatment period (FIG. 2A). Tumor growth and bodyweights of animals were monitored during and post-treatment. Subject mice remained on study until tumor size reached 1.5 cm³, clinical signs were observed, or to the study end date. Mice in the AZD4625 monotherapy and combination groups were monitored up to 65 days post-implant while mice in the Compound of Formula IV monotherapy and combination groups were monitored up to 119 days post-implant.

Result: Treatment group treated with Compound of Formula IV monotherapy initially showed tumor regression similar to the AZD4625 monotherapy treated group, but 8 out of 9 tumors returned after drug cessation (FIG. 2B, “Vehicle,” “AZD4625,” “Compound of Formula IV,” “AZD6738”). In contrast, the treatment group treated with Compound of Formula IV and AZD6738 combination therapy showed complete tumor regression with responses being durable off drug, with 9 out of 9 animals maintaining the tumor regression level even after drug cessation and for the monitoring period of 90 days afterwards (FIG. 2B, “Compound of Formula IV+AZD6738”). In the AZD6738 and AZD4625 group, 7/9 mice showed complete responses, and these were sustained in 5 of the mice when drug treatment was stopped (FIG. 2B, “AZD4625+AZD6738”).

### Example 3

The long-term anti-tumor activity through dual inhibition of KRAS and ATR in KRAS mutant cancers was determined in a follow-up, in vivo study (rechallenge study) using KRAS^{G12C} and ATR inhibitors. The KRAS^{G12C} and ATR inhibitors used were Compound of Formula IV and AZD6738.

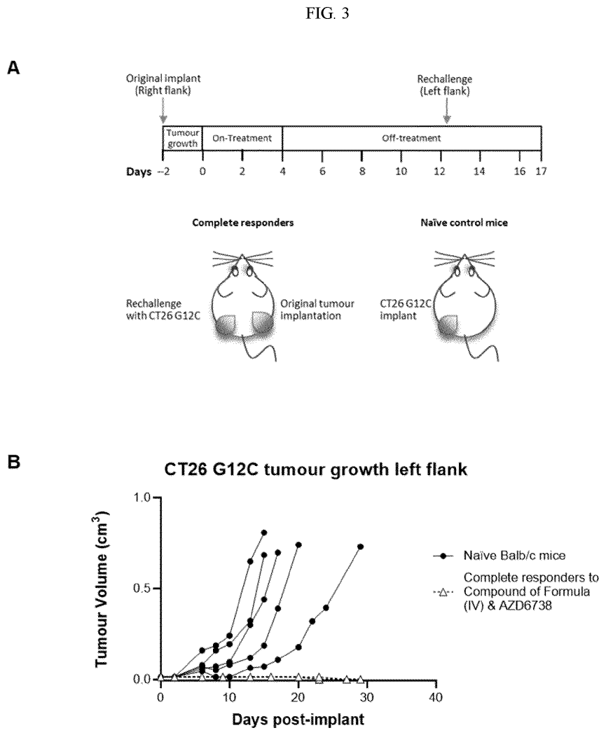

For the rechallenge study, 5×10⁵ CT26^{G12C} (G12C clone P5E6) cells were implanted subcutaneously on the left flank of five mice that had shown complete anti-tumor responses to Compound of Formula IV and AZD6738 combination therapy. For the control group, 5×10⁵ CT26^{G12C} (G12C clone P5E6) cells were implanted on the left flank of naïve female BALB/c mice (FIG. 3A). Tumor engraftment and growth was monitored for 4 weeks with no drug treatments given.

Result: CT26^{G12C} tumor growth was observed as expected in the naïve control mice, but no tumor growth was observed on either flank of the rechallenge mice group (FIG. 3B). A lesion at a non-implant site (lymph node) was observed in one mouse in the rechallenge group, but it could not be confirmed that the origin of said tumor was the implanted CT26^{G12C} cells. This data suggests that mice treated with KRAS^{G12C} and ATR inhibitor combination therapy may have developed a long term anti-tumor immunity.

### Example 4

The adaptive immune system's role in driving the anti-tumor activity in dual inhibition of KRAS and ATR in KRAS mutant cancers was determined using CT26^{G12C} xenograft studies performed in nude mice lacking a thymus and are thus unable to generate mature T-cells.

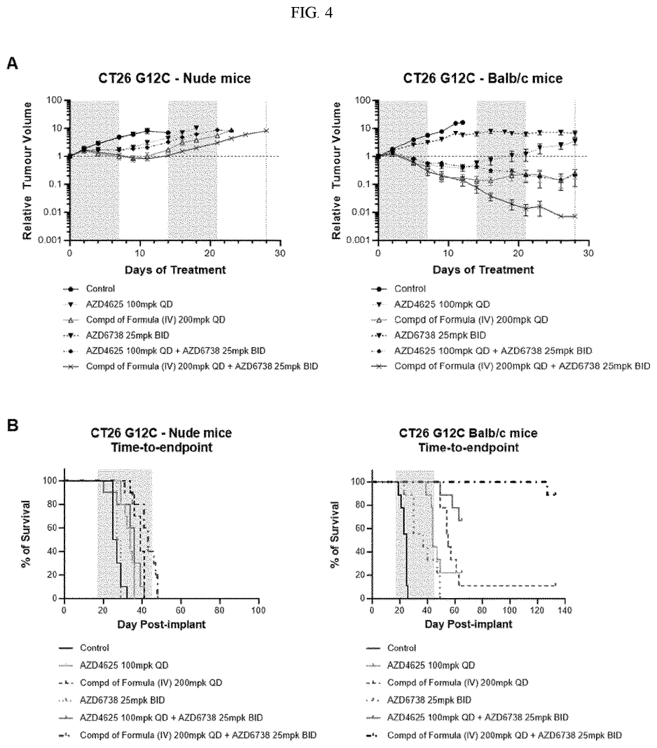

Result: Transient regression of the CT26^{G12C} tumors were observed with KRAS^{G12C} inhibitors monotherapy in the nude mice, although the depth and duration of the anti-tumor response was less robust than in immunocompetent BALB/c mice (FIG. 4A). Anti-tumor responses to KRAS^{G12C} inhibitors and AZD6738 combination therapy were modestly better than KRAS^{G12C} inhibitors monotherapy in the nude mice (FIG. 4A). However, in contrast to the deep and durable regression of CT26^{G12C} tumors observed in the immunocompetent BALB/c mice, there were no complete responders to the combination therapy in the nude mice (FIG. 4A).

The lower anti-tumor activity in the nude mice translated into lower overall survival rate with all the test subjects coming off the study within 2 days of the treatment period due to tumor volume or clinical signs (FIG. 4B). Therefore, this data suggests that the deep and durable anti-tumor activity observed with KRAS^{G12C} inhibitors and AZD6738 is being mediated through an adaptive immune response.

KRAS^{G12C} inhibitors and AZD6738 combination therapies show both unexpected and robust anti-tumor activity against preclinical models of KRAS^{G12C} mutant cancer in mice with intact immune systems. Complete tumor regressions are observed with combination therapies and these regressions are durable following the cessation of drug treatment. Mice subjects showing complete anti-tumor response to the combination therapy were also protected from tumor rechallenge, suggesting a long-term memory response against the tumor has been induced. This data is also consistent with the hypothesis that the deep and durable anti-tumor activity of the KRAS^{G12C} inhibitors and AZD6738 combination therapy is being mediated through an adaptive immune response.

### Example 5

Additional anti-tumor studies using KRAS inhibitors adagrasib (MTRX849) or sotorasib (AMG510) in combination with AZD6738 (ceralasertib) ATR inhibitor were performed using in vivo CT26^{G12C} xenograft studies.

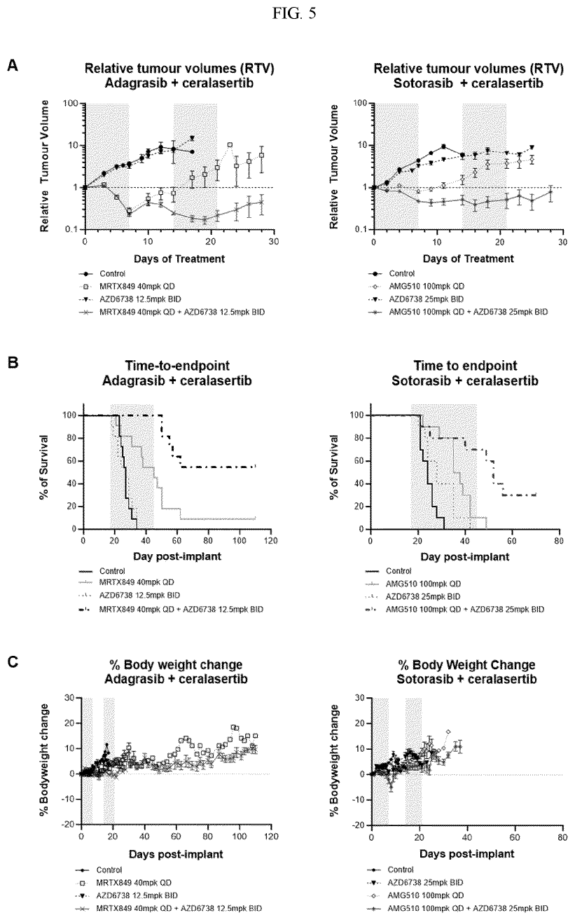

CT26^{G12C} murine colon carcinoma cells were grown subcutaneously in BALB/c mice with an intact immune system as described in Examples 1 and 2. Subject mice were randomized into relevant treatment and control groups and treated with vehicle control, adagrasib, sotorasib or AZD6738 in monotherapy, or in combination therapy for 4 weeks. Adagrasib or sotorasib was dosed daily and AZD6738 was dosed twice daily on a 7 day-on, 7 day-off schedule at the indicated doses for the treatment period (FIG. 5). Tumor growth and body weights of animals were monitored during and post-treatment. Subject mice remained on study until tumor size reached 1.5 cm³, clinical signs were observed, or to the study end.

Result: Treatment groups with adagrasib or sotorasib monotherapy showed initial tumor growth delay (FIG. 5A) and increased survival rates (FIG. 5B) compared to the vehicle control group, but with most tumors returning after drug cessation and 0-10% of mice remaining at study end. AZD6738 monotherapy showed no or weak activity compared to the vehicle control group. In contrast, both the adagrasib and AZD6738 or sotorasib and AZD6738 combination treatment groups showed improved and more durable tumor growth inhibition compared to their respective KRAS^{G12C} inhibitor monotherapy groups (FIG. 5A), and with further extended survival rates (FIG. 5B) with 30-50% mice remaining at study end. All treatments were well tolerated with minimal impact on body weights compared to vehicle control groups (FIG. 5C).

What is claimed is:

1. A method of treating cancer in a subject, the method comprising administering to the subject:
a. a KRAS inhibitor, and
b. an Ataxia Telangiectasia and Rad-3-related (ATR) inhibitor.

2. A KRAS inhibitor for use in the treatment of cancer in a subject, wherein said treatment comprises the separate, sequential or simultaneous administration of:
a. said KRAS inhibitor, and
b. an ATR inhibitor,
to said subject.

3. An ATR inhibitor for use in the treatment of cancer in a subject, wherein said treatment comprises the separate, sequential or simultaneous administration of:
a. a KRAS inhibitor, and
b. said ATR inhibitor,
to said subject.

4. Use of a KRAS inhibitor or an ATR inhibitor in the manufacture of a medicament for administration of the KRAS inhibitor and the ATR inhibitor in combination, for the treatment of cancer in a subject.

5. The method of claim 1, inhibitor for use of claim 2 or claim 3, or use of claim 4, wherein the KRAS inhibitor is an antibody.

6. The method, inhibitor for use, or use of claim 5, wherein the antibody is ELI-002, KRAS-EphA-2-CAR-DC, Anti-KRAS G12V mTCR PBL, anti-KRAS G12D mTCR PBL, siG12D-LODER, or KRAS G12D siRNA.

7. The method, inhibitor for use or use of any one of claims 1 to 5, wherein the KRAS inhibitor is a small molecule KRAS inhibitor.

8. The method, inhibitor for use or use of claim 7, wherein the small molecule KRAS inhibitor is sotorasib, adagrasib, Ly3537982, GDC-6036, D-1553, JDQ443, BI1823911, GFH925, D35-001, JAB-21822, HBI-2438, YL-15293, GEC255, IB1351, RMC-6291, MK-1084, MRTX1133, HRS-4642, RMC-9805, JAB-2200, Vrtx153, ERAS-4, ASP3082, RMC-0708, RMC-8839, RMC-6291, JAB-23400, TEB-17231, or QTX3046.

9. The method, inhibitor for use or use of claim 7, wherein the small molecule KRAS inhibitor is a compound of Formula (I): 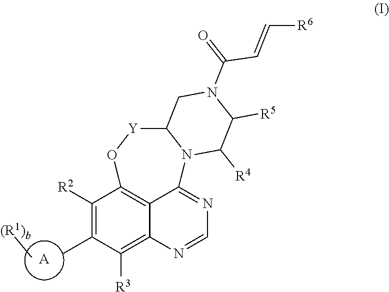
wherein:
Ring A is selected from phenyl and bicyclic heteroaryl;
R¹ in each occurrence is independently selected from C₁₋₄ alkyl, halo, hydroxy, C₁₋₄ alkoxy, C₁₋₃ fluoroalkyl, C₁₋₃ fluoroalkoxy, cyano and acetylenyl;
b is 0, 1, 2 or 3;
Y is CH₂ or CH₂CH₂;
R² is cyano, halo, C₁₋₄alkyl, C₁₋₄alkoxy or C₁₋₃ fluoroalkyl;
R³ is F, Me, Et, MeO or C₁₋₂ fluoroalkyl;
R⁴ is H or Me;
R⁵ is H or Me;
R⁶ is H or CH₂NMe₂;
or a pharmaceutically acceptable salt thereof, provided that when Y is CH₂, R² is Cl, R³ is F, ring A is phenyl, b is 2, the R¹ groups are F and OH and are each ortho to the biaryl bond, and when both R⁴ and R⁶ are H, then R⁵ is Me.

10. The method, inhibitor for use or use of claim 9, wherein the small molecule KRAS inhibitor has the structure

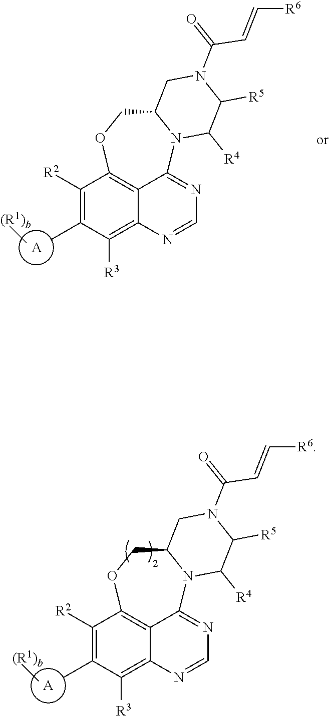

11. The method, inhibitor for use or use of claim 10, wherein the ring A is selected from the group consisting of: 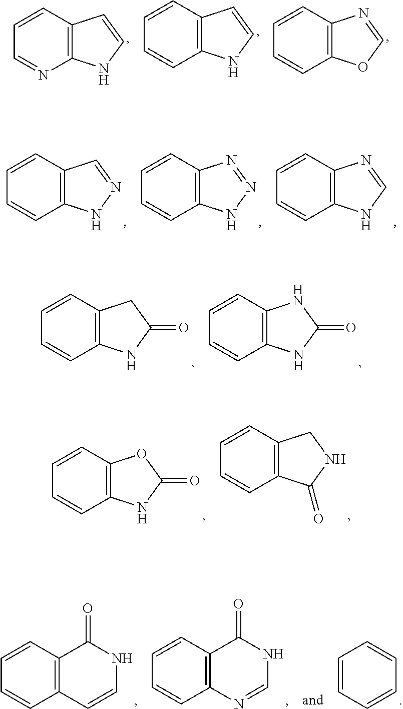
wherein the ring A is attached to the remainder of compound of Formula (I) at any point on the above listed rings.

12. The method, inhibitor for use or use of any one of claims 9 to 11, wherein R⁴ is H.

13. The method, inhibitor for use or use of any one of claims 9 to 12, wherein R⁶ is H.

14. The method, inhibitor for use or use of claim 9, wherein Y is CH₂.

15. The method, inhibitor for use or use of claim 9, wherein Y is CH₂CH₂.

16. The method, inhibitor for use or use of any one of claims 9 to 15, wherein R² is Cl.

17. The method, inhibitor for use or use of any one of claims 9 to 16, wherein R³ is F.

18. The method, inhibitor for use or use of any one of claims 9 to 17, wherein R⁴ is H and R⁵ is Me.

19. The method, inhibitor for use or use of claim 9, wherein the small molecule KRAS inhibitor is selected from:
7-[(8aS)-10-Acryloyl-6-chloro-4-fluoro-8,8a,9,10,11,12-hexahydropyrazino[2′,1′:3,4][1,4]oxazepino [5,6,7-de]quinazolin-5-yl]-6-methyl-2,3-dihydro-1H-isoindol-1-one;
1-[(8aS,11S)-6-Chloro-4-fluoro-5-(2-fluoro-6-hydroxyphenyl)-11-methyl-8a,9,11,12-tetrahydropyrazino[2′,1′:3,4][1,4]oxazepino[5,6,7-de]quinazolin-10(8H)-yl]prop-2-en-1-one;
1-[(8aS,11R)-6-Chloro-4-fluoro-5-(2-fluoro-6-hydroxyphenyl)-11-methyl-8a,9,11,12-tetrahydropyrazino[2′,1′:3,4][1,4]oxazepino[5,6,7-de]quinazolin-10(8H)-yl]prop-2-en-1-one;
5-[(8aS)-10-Acryloyl-6-chloro-4-fluoro-8,8a,9,10,11,12-hexahydropyrazino[2′,1′:3,4][1,4]oxazepino [5,6,7-de]quinazolin-5-yl]-6-methylquinazolin-4(3H)-one;
1-[(8aS)-6-Chloro-4-fluoro-5-(5-methyl-1H-benzimidazol-4-yl)-8a,9,11,12-tetrahydropyrazino [2′,1′:3,4][1,4]oxazepino[5,6,7-de]quinazolin-10(8H)-yl]prop-2-en-1-one;
8-[(8aS)-6-Chloro-4-fluoro-10-(prop-2-enoyl)-8,8a,9,10,11,12-hexahydropyrazino[2′,1′:3,4][1,4]oxazepino[5,6,7-de]quinazolin-5-yl]isoquinolin-1(2H)-one;
1-[(8aS)-6-Chloro-4-fluoro-5-(1H-indazol-3-yl)-8a,9,11,12-tetrahydropyrazino[2′,1′:3,4][1,4]oxazepino[5,6,7-de]quinazolin-10(8H)-yl]prop-2-en-1-one;
1-[(8aS)-6-Chloro-4-fluoro-5-(2-hydroxy-6-methylphenyl)-8a,9,11,12-tetrahydropyrazino[2′,1′:3,4][1,4]oxazepino[5,6,7-de]quinazolin-10(8H)-yl]prop-2-en-1-one;
(2E)-1-[(8aS)-6-Chloro-4-fluoro-5-(2-fluoro-6-hydroxyphenyl)-8a,9,11,12-tetrahydropyrazino [2′,1′:3,4][1,4]oxazepino[5,6,7-de]quinazolin-10(8H)-yl]-4-(dimethylamino)but-2-en-1-one;
8-[(8aS)-6-Chloro-4-fluoro-10-(prop-2-enoyl)-8,8a,9,10,11,12-hexahydropyrazino[2′,1′:3,4][1,4]oxazepino[5,6,7-de]quinazolin-5-yl]-7-methylisoquinolin-1(2H)-one;
1-[(8aS)-6-Chloro-4-fluoro-5-(5-methyl-1H-benzotriazol-4-yl)-8a,9,11,12-tetrahydropyrazino [2′,1′:3,4][1,4]oxazepino[5,6,7-de]quinazolin-10(8H)-yl]prop-2-en-1-one;
1-((8aS)-6-chloro-4-fluoro-5-(5-fluoro-1H-benzo[d]imidazol-4-yl)-8a,9,11,12-tetrahydropyrazino [2′,1′:3,4][1,4]oxazepino[5,6,7-de]quinazolin-10(8H)-yl)prop-2-en-1-one;
1-[(8aS)-6-Chloro-4-fluoro-5-(5-fluoro-1H-indazol-4-yl)-8a,9,11,12-tetrahydropyrazino[2′,1′:3,4][1,4]oxazepino[5,6,7-de]quinazolin-10(8H)-yl]prop-2-en-1-one;
1-((8aS)-6-Chloro-4-fluoro-5-(5-fluoro-1-methyl-1H-benzo[d]imidazol-4-yl)-8a,9,11,12-tetrahydropyrazino[2′,1′:3,4][1,4]oxazepino[5,6,7-de]quinazolin-10(8H)-yl)prop-2-en-1-one;
1-[(8aS)-6-Chloro-4-fluoro-5-(5-fluoro-1H-benzotriazol-4-yl)-8a,9,11,12-tetrahydropyrazino[2′,1′:3,4][1,4]oxazepino[5,6,7-de]quinazolin-10(8H)-yl]prop-2-en-1-one;
8-[(8aS)-6-Chloro-4-fluoro-10-(prop-2-enoyl)-8,8a,9,10,11,12-hexahydropyrazino[2′,1′:3,4][1,4]oxazepino[5,6,7-de]quinazolin-5-yl]-7-fluoroisoquinolin-1(2H)-one;
(2E)-1-[(8aS)-6-chloro-4-fluoro-5-(5-methyl-1H-indazol-4-yl)-8a,9,11,12-tetrahydropyrazino [2′,1′:3,4][1,4]oxazepino[5,6,7-de]quinazolin-10(8H)-yl]-4-(dimethylamino)but-2-en-1-one;
1-[(6aR,9S)-3-Chloro-1-fluoro-2-(2-fluoro-6-hydroxyphenyl)-9-methyl-5,6,6a,7,9,10-hexahydro-8H-pyrazino[1′,2′:5,6][1,5]oxazocino[4,3,2-de]quinazolin-8-yl]prop-2-en-1-one;
1-[(6aR,9S)-3-Chloro-1-fluoro-2-(2-fluoro-6-hydroxyphenyl)-9-methyl-5,6,6a,7,9,10-hexahydro-8H-pyrazino[1′,2′:5,6][1,5]oxazocino[4,3,2-de]quinazolin-8-yl]prop-2-en-1-one;
8-[3-Chloro-1-fluoro-8-(prop-2-enoyl)-6,6a,7,8,9,10-hexahydro-5H-pyrazino[1′,2′:5,6][1,5]oxazocino[4,3,2-de]quinazolin-2-yl]-7-methylisoquinolin-1(2H)-one;
1-[(6aS,9R)-3-Chloro-1-fluoro-2-(2-fluoro-6-hydroxyphenyl)-9-methyl-5,6,6a,7,9,10-hexahydro-8H-pyrazino[1′,2′:5,6][1,5]oxazocino[4,3,2-de]quinazolin-8-yl]prop-2-en-1-one;
1-[(6aR,9R)-3-Chloro-1-fluoro-2-(2-fluoro-6-hydroxyphenyl)-9-methyl-5,6,6a,7,9,10-hexahydro-8H-pyrazino[1′,2′:5,6][1,5]oxazocino[4,3,2-de]quinazolin-8-yl]prop-2-en-1-one;
1-[(6aS,9S)-3-Chloro-1-fluoro-2-(2-fluoro-6-hydroxyphenyl)-9-methyl-5,6,6a,7,9,10-hexahydro-8H-pyrazino[1′,2′:5,6][1,5]oxazocino[4,3,2-de]quinazolin-8-yl]prop-2-en-1-one;
1-[(8aS)-4-Chloro-6-fluoro-5-(2-fluoro-6-hydroxyphenyl)-8a,9,11,12-tetrahydropyrazino[2′,1′:3,4][1,4]oxazepino[5,6,7-de]quinazolin-10(8H)-yl]prop-2-en-1-one;
1-[(8aS,11R)-6-Chloro-4-fluoro-11-methyl-5-(5-methyl-1H-benzimidazol-4-yl)-8a,9,11,12-tetrahydropyrazino[2′,1′:3,4][1,4]oxazepino[5,6,7-de]quinazolin-10(8H)-yl]prop-2-en-1-one;
8-[(8aS,11R)-6-Chloro-4-fluoro-11-methyl-10-(prop-2-enoyl)-8,8a,9,10,11,12-hexahydropyrazino [2′,1′:3,4][1,4]oxazepino[5,6,7-de]quinazolin-5-yl]-7-methylisoquinolin-1(2H)-one;
(2E)-1-[(8aS,11R)-6-Chloro-4-fluoro-11-methyl-5-(5-methyl-1H-benzimidazol-4-yl)-8a,9,11,12-tetrahydropyrazino[2′,1′:3,4][1,4]oxazepino[5,6,7-de]quinazolin-10(8H)-yl]-4-(dimethylamino)but-2-en-1-one; and
(2E)-1-[(8aS,11R)-6-Chloro-4-fluoro-11-methyl-5-(5-methyl-1H-indazol-4-yl)-8a,9,11,12-tetrahydropyrazino[2′,1′:3,4][1,4]oxazepino[5,6,7-de]quinazolin-10(8H)-yl]-4-(dimethylamino)but-2-en-1-one;
or a pharmaceutically acceptable salt thereof.

20. The method, inhibitor for use or use of claim 7, wherein the small molecule KRAS inhibitor is a compound of Formula (II):

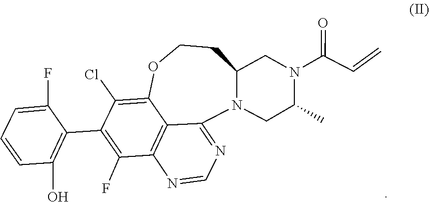 [SMILES: C=CC(=O)N1C[C@@H]2CCOc3c(Cl)c(-c4c(O)cccc4F)c(F)c4ncnc(c34)N2C[C@H]1C]

21. The method, inhibitor for use or use of claim 7, wherein the small molecule KRAS inhibitor is a compound of Formula III: 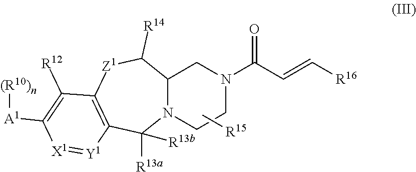
wherein:
A¹ is phenyl or a bicyclic heteroaryl group;
X¹ and Y¹ are connected by a double bond and i) X¹ is CR¹⁷ and Y¹ is CR¹⁸, ii) X¹ is N and Y¹ is CR¹⁸, or iii) X¹ is CR¹⁷ and Y¹ is N; or
X¹ and Y¹ together are C(O)NR¹⁹; or
X¹ and Y¹ are adjacent ring atoms of an optionally substituted 5- or 6-membered N-heterocycle fused to the aromatic ring substituted with Z¹, and X¹ and Y¹ are both C or are C and N;
Z¹ is O, NH, or NMe;
R¹⁰ is independently selected from F, Cl, Br, OH, CH₂OH, OMe, CH₂OMe, C₁-C₃ alkyl and C₁-C₃ fluoroalkyl;
n is 0, 1, 2 or 3;
R¹² is H, F, Cl, CCH, CCMe, CN, Br, C₁-C₃ alkyl, C₁-C₃ fluoroalkyl, OMe or OEt;
R^{13a} and R^{13b} together are ═O or R^{13a} and R^{13b} are H;
R¹⁴ is H or Me;
R¹⁵ is H or Me;
R¹⁶ is H or CH₂NMe₂;
R¹⁷ and R¹⁸ are independently selected from H, F, Cl, CCH, CC(C₁-C₃ alkyl), CCCH₂Nme₂, CCCH₂O(C₁-C₃ alkyl), CN, Me, C₁-C₆alkyl, OH, OMe, O(C₁-C₃ alkyl), O(C₁-C₃ deuteroalkyl), O(C₁-C₃ fluoroalkyl), O(C₃-C₆ cycloalkyl), C₁-C₃ fluoroalkyl, OCH₂CH₂NMe₂, OCH₂CH₂OMe, CH₂OMe, OCH₂CH₂N(CH₂CH₂)₂CH, OCH₂CH₂N(CH₂CH₂)₂O, OCH₂CH₂(2-pyridyl) or an optionally substituted 3-, 4-, 5- or 6-membered carbocycle or heterocycle; or
R¹⁷ and R¹⁸ combine to form an optionally substituted 5- or 6-membered carbocycle or heterocycle;
R¹⁹ is selected from H, Me, Et, C₃H₇ and C₁-C₃ fluoroalkyl;
or a pharmaceutically acceptable salt thereof.

22. The method, inhibitor for use or use of claim 21, wherein i) X¹ is CR¹⁷ and Y¹ is CR¹⁸, ii) X¹ is N and Y¹ is CR¹⁸ or iii) X¹ is CR¹⁷ and Y¹ is N.

23. The method, inhibitor for use or use of claim 21 or 22, wherein Z¹ is O.

24. The method, inhibitor for use or use of any one of claims 21 to 23, wherein R^{13a} and R^{13b} are H.

25. The method, inhibitor for use or use of any one of claims 21 to 24, wherein R¹⁴ is H.

26. The method, inhibitor for use or use of any one of claims 21 to 25, wherein R¹⁶ is H.

27. The method, inhibitor for use or use of any one of claims 21 to 26, wherein A¹ is phenyl.

28. The method, inhibitor for use or use of claim 21, wherein the small molecule KRAS inhibitor is selected from:
(12aS)-2-Acryloyl-10-chloro-9-(5-methyl-1H-indazol-4-yl)-1,2,3,4,12,12a-hexahydro-6H-benzo[f]pyrazino[2,1-c][1,4]oxazepin-6-one;
1-((12aS)-10-Chloro-9-(5-methyl-1H-indazol-4-yl)-3,4,12,12a-tetrahydro-6H-benzo[f]pyrazino[2,1-c][1,4]oxazepin-2(1H)-yl)prop-2-en-1-one;
1-[(12aR)-10-Chloro-9-(2-fluoro-6-hydroxyphenyl)-7-methoxy-3,4,12,12a-tetrahydro-6H-pyrazino[2,1-c][1,4]benzoxazepin-2(1H)-yl]prop-2-en-1-one;
(12aR)-10-chloro-9-(2-fluoro-6-hydroxyphenyl)-7-hydroxy-2-(prop-2-enoyl)-1,2,3,4,12,12a-hexahydro-6H-pyrazino[2,1-c][1,4]benzoxazepin-6-one;
(12aR)-10-Chloro-9-(5-methyl-1H-indazol-4-yl)-2-(prop-2-enoyl)-1,2,3,4,12,12a-hexahydro-6H-pyrazino[2,1-c][1,4]benzoxazepin-6-one;
1-((12aR)-10-Chloro-9-(5-methyl-1H-indazol-4-yl)-3,4,12,12a-tetrahydro-6H-benzo[f]pyrazino[2,1-c][1,4]oxazepin-2(1H)-yl)prop-2-en-1-one;
(12aR)-10-Chloro-8-fluoro-9-(2-fluoro-6-hydroxyphenyl)-2-(prop-2-enoyl)-1,2,3,4,12,12a-hexahydro-6H-pyrazino[2,1-c][1,4]benzoxazepin-6-one;
1-[(12aR)-8,10-dichloro-9-(2-fluoro-6-hydroxyphenyl)-7-hydroxy-3,4,12,12a-tetrahydro-6H-pyrazino[2,1-c][1,4]benzoxazepin-2(1H)-yl]prop-2-en-1-one;
1-[(12aR)-10-Chloro-9-(2-fluoro-6-hydroxyphenyl)-7-(1H-imidazol-1-yl)-3,4,12,12a-tetrahydro-6H-pyrazino[2,1-c][1,4]benzoxazepin-2(1H)-yl]prop-2-en-1-one;
(12aR)-10-Chloro-9-(2-fluoro-6-hydroxyphenyl)-2-(prop-2-enoyl)-1,2,3,4,12,12a-hexahydro-6H-pyrazino[2,1-c][1,4]benzoxazepine-7-carbonitrile;
1-[(12aR)-10-Chloro-9-(2-fluoro-6-hydroxyphenyl)-7-(1H-pyrazol-1-yl)-3,4,12,12a-tetrahydro-6H-pyrazino[2,1-c][1,4]benzoxazepin-2(1H)-yl]prop-2-en-1-one;
1-((12aR)-10-Chloro-8-fluoro-9-(5-methyl-1H-benzo[d]imidazol-4-yl)-3,4,12,12a-tetrahydro-6H-benzo[f]pyrazino[2,1-c][1,4]oxazepin-2(1H)-yl)prop-2-en-1-one;
(12aR)-8,10-Dichloro-9-(2-fluoro-6-hydroxyphenyl)-2-(prop-2-enoyl)-1,2,3,4,12,12a-hexahydro-6H-pyrazino[2,1-c][1,4]benzoxazepin-6-one;
(12aR)-10-Chloro-9-(2-fluoro-6-hydroxyphenyl)-2-(prop-2-enoyl)-1,2,3,4,12,12a-hexahydro-6H-pyrazino[2,1-c][1,4]benzoxazepine-8-carbonitrile;
(12aR)-10-Chloro-9-(2-fluoro-6-hydroxyphenyl)-8-methyl-2-(prop-2-enoyl)-1,2,3,4,12,12a-hexahydro-6H-pyrazino[2,1-c][1,4]benzoxazepin-6-one;
1-[(12aR)-8,10-Dichloro-9-(2-fluoro-6-hydroxyphenyl)-3,4,12,12a-tetrahydro-6H-pyrazino[2,1-c][1,4]benzoxazepin-2(1H)-yl]prop-2-en-1-one;
8-[(12aR)-10-Chloro-8-fluoro-2-(prop-2-enoyl)-1,2,3,4,12,12a-hexahydro-6H-pyrazino[2,1-c][1,4]benzoxazepin-9-yl]-7-methylisoquinolin-1(2H)-one;
1-[(12aR)-10-chloro-9-(2-fluoro-6-hydroxyphenyl)-8-methoxy-3,4,12,12a-tetrahydro-6H-pyrazino[2,1-c][1,4]benzoxazepin-2(1H)-yl]prop-2-en-1-one;
(12aS)-10-Chloro-9-(5-methyl-1H-indazol-4-yl)-2-(prop-2-enoyl)-1,3,4,11,12,12a-hexahydropyrazino[2,1-c][1,4]benzodiazepin-6(2H)-one;
1-[(12aR)-10-chloro-9-(2-chloro-6-hydroxyphenyl)-8-fluoro-3,4,12,12a-tetrahydro-6H-pyrazino[2,1-c][1,4]benzoxazepin-2(1H)-yl]prop-2-en-1-one;
(12aS)-10-Chloro-11-methyl-9-(5-methyl-1H-indazol-4-yl)-2-(prop-2-enoyl)-1,3,4,11,12,12a-hexahydropyrazino[2,1-c][1,4]benzodiazepin-6(2H)-one;
1-[(12aR)-10-Chloro-9-(2,3-difluoro-6-hydroxyphenyl)-8-fluoro-3,4,12,12a-tetrahydro-6H-pyrazino[2,1-c][1,4]benzoxazepin-2(1H)-yl]prop-2-en-1-one;
(12aR)-10-Chloro-9-(2-hydroxy-6-methylphenyl)-2-(prop-2-enoyl)-1,2,3,4,12,12a-hexahydro-6H-pyrazino[2,1-c][1,4]benzoxazepine-8-carbonitrile;
1-[(12aR)-9-(2-Chloro-6-hydroxyphenyl)-8,10-difluoro-3,4,12,12a-tetrahydro-6H-pyrazino[2,1-c][1,4]benzoxazepin-2(1H)-yl]prop-2-en-1-one;
1-[(12aR)-8,10-Difluoro-9-(2-hydroxy-6-methylphenyl)-3,4,12,12a-tetrahydro-6H-pyrazino[2,1-c][1,4]benzoxazepin-2(1H)-yl]prop-2-en-1-one;
1-[(12aR)-8,10-Difluoro-9-[2-fluoro-6-(hydroxymethyl)phenyl]-3,4,12,12a-tetrahydro-6H-pyrazino[2,1-c][1,4]benzoxazepin-2(1H)-yl]prop-2-en-1-one;
1-[(12aR)-8,10-Difluoro-9-[2-hydroxy-6-(trifluoromethyl)phenyl]-3,4,12,12a-tetrahydro-6H-pyrazino[2,1-c][1,4]benzoxazepin-2(1H)-yl]prop-2-en-1-one;
1-[(12aR)-9-(2-Ethyl-6-hydroxyphenyl)-8,10-difluoro-3,4,12,12a-tetrahydro-6H-pyrazino[2,1-c][1,4]benzoxazepin-2(1H)-yl]prop-2-en-1-one;
1-[(12aR)-9-[2-(Difluoromethyl)-6-hydroxyphenyl]-8,10-difluoro-3,4,12,12a-tetrahydro-6H-pyrazino[2,1-c][1,4]benzoxazepin-2(1H)-yl]prop-2-en-1-one;
(12aR)-9-(2-Chloro-6-hydroxyphenyl)-10-fluoro-2-(prop-2-enoyl)-1,2,3,4,12,12a-hexahydro-6H-pyrazino[2,1-c][1,4]benzoxazepine-8-carbonitrile;
(12aR)-10-Chloro-9-(2-chloro-6-hydroxyphenyl)-2-(prop-2-enoyl)-1,2,3,4,12,12a-hexahydro-6H-pyrazino[2,1-c][1,4]benzoxazepine-8-carbonitrile;
1-[(12aR)-9-(2-Bromo-6-hydroxyphenyl)-8,10-difluoro-3,4,12,12a-tetrahydro-6H-pyrazino[2,1-c][1,4]benzoxazepin-2(1H)-yl]prop-2-en-1-one;
1-[(12aR)-8-Chloro-10-fluoro-9-(2-hydroxy-6-methylphenyl)-3,4,12,12a-tetrahydro-6H-pyrazino[2,1-c][1,4]benzoxazepin-2(1H)-yl]prop-2-en-1-one;
1-[(12aR)-8-Chloro-10-ethynyl-9-(2-hydroxy-6-methylphenyl)-3,4,12,12a-tetrahydro-6H-pyrazino[2,1-c][1,4]benzoxazepin-2(1H)-yl]prop-2-en-1-one;
1-[(12aR)-10-Ethynyl-8-fluoro-9-(2-hydroxy-6-methylphenyl)-3,4,12,12a-tetrahydro-6H-pyrazino[2,1-c][1,4]benzoxazepin-2(1H)-yl]prop-2-en-1-one;
(6aR)-4-Chloro-3-(2-fluoro-6-hydroxyphenyl)-2-methyl-8-(prop-2-enoyl)-2,6,6a,7,8,9,10,12-octahydro-1H-pyrazino[2,1-c]pyrido[3,4-f][1,4]oxazepin-1-one;
1-[(6aR)-1,4-Dichloro-3-(2-fluoro-6-hydroxyphenyl)-6a,7,9,10-tetrahydro-12H-pyrazino[2,1-c]pyrido[3,4-J][1,4]oxazepin-8(6H)-yl]prop-2-en-1-one;
(6aR)-4-Chloro-3-(2-fluoro-6-hydroxyphenyl)-8-(prop-2-enoyl)-2,6,6a,7,8,9,10,12-octahydro-1H-pyrazino[2,1-c]pyrido[3,4-f][1,4]oxazepin-1-one;
1-[(8aR)-6-Chloro-5-(2-fluoro-6-hydroxyphenyl)-8a,9,11,12-tetrahydro-14H-pyrazino[2,1-c][1,2,4]triazolo[4′,3′:1,2]pyrido[3,4-f][1,4]oxazepin-10(8H)-yl]prop-2-en-1-one;
1-[(7aR)-5-chloro-4-(2-fluoro-6-hydroxyphenyl)-1-methyl-1,7a,8,10,11,13-hexahydropyrazino[2′,1′:3,4][1,4]oxazepino[7,6-g]indazol-9(7H)-yl]prop-2-en-1-one; and
1-[(7aR)-5-Chloro-4-(2-fluoro-6-hydroxyphenyl)-2-methyl-2,7a,8,10,11,13-hexahydropyrazino[2′,1′:3,4][1,4]oxazepino[7,6-g]indazol-9(7H)-yl]prop-2-en-1-one;
1-[(12aR)-9-(2-chloro-6-hydroxyphenyl)-8-ethynyl-10-fluoro-3,4,12,12a-tetrahydro-6H-pyrazino[2,1-c][1,4]benzoxazepin-2(1H)-yl]prop-2-en-1-one;
1-[(12aR)-10-Chloro-9-(2-chloro-6-hydroxyphenyl)-8-ethynyl-3,4,12,12a-tetrahydro-6H-pyrazino[2,1-c][1,4]benzoxazepin-2(1H)-yl]prop-2-en-1-one;
1-((12aR)-10-chloro-8-ethynyl-9-(2-fluoro-6-hydroxyphenyl)-3,4,12,12a-tetrahydro-6H-benzo[f]pyrazino[2,1-c][1,4]oxazepin-2(1H)-yl)prop-2-en-1-one;
1-[(7aR)-5-Chloro-4-(2-chloro-6-hydroxyphenyl)-1-methyl-1,7a,8,10,11,13-hexahydroimidazo[4,5-g]pyrazino[2,1-c][1,4]benzoxazepin-9(7H)-yl]prop-2-en-1-one;
1-[(12aR)-8-Chloro-9-(2-chloro-6-hydroxyphenyl)-10-fluoro-3,4,12,12a-tetrahydro-6H-pyrazino[2,1-c][1,4]benzoxazepin-2(1H)-yl]prop-2-en-1-one;
1-[(12aR)-9-(2-Chloro-6-hydroxyphenyl)-10-fluoro-8-(prop-1-yn-1-yl)-3,4,12,12a-tetrahydro-6H-pyrazino[2,1-c][1,4]benzoxazepin-2(1H)-yl]prop-2-en-1-one;
1-[(6aR)-4-Chloro-3-(2-chloro-6-hydroxyphenyl)-2-ethynyl-6a,7,9,10-tetrahydro-12H-pyrazino[2,1-c]pyrido[2,3-][1,4]oxazepin-8(6H)-yl]prop-2-en-1-one;
1-[(12aR)-9-(2-Chloro-6-hydroxyphenyl)-8-ethynyl-10-methyl-3,4,12,12a-tetrahydro-6H-pyrazino[2,1-c][1,4]benzoxazepin-2(1H)-yl]prop-2-en-1-one;
1-[(12aR)-10-chloro-9-(2-chloro-6-hydroxyphenyl)-7,8-difluoro-3,4,12,12a-tetrahydro-6H-pyrazino[2,1-c][1,4]benzoxazepin-2(1H)-yl]prop-2-en-1-one;
1-[(12aR)-9-(2-chloro-6-hydroxyphenyl)-8-(difluoromethoxy)-10-fluoro-3,4,12,12a-tetrahydro-6H-pyrazino[2,1-c][1,4]benzoxazepin-2(1H)-yl]prop-2-en-1-one;
1-[(12aR)-9-(2-chloro-6-hydroxyphenyl)-8-ethynyl-7,10-difluoro-3,4,12,12a-tetrahydro-6H-pyrazino[2,1-c][1,4]benzoxazepin-2(1H)-yl]prop-2-en-1-one;
1-[(12aR)-10-Chloro-9-(2-chloro-6-hydroxyphenyl)-8-(difluoromethoxy)-3,4,12,12a-tetrahydro-6H-pyrazino[2,1-c][1,4]benzoxazepin-2(1H)-yl]prop-2-en-1-one;
1-[(12aR)-9-(2-chloro-6-hydroxyphenyl)-8-(cyclopropyloxy)-10-fluoro-3,4,12,12a-tetrahydro-6H-pyrazino[2,1-c][1,4]benzoxazepin-2(1H)-yl]prop-2-en-1-one;
1-((12aR)-9-(2-Chloro-6-hydroxyphenyl)-8-(3-(dimethylamino)prop-1-yn-1-yl)-10-fluoro-3,4,12,12a-tetrahydro-6H-benzo[f]pyrazino[2,1-c][1,4]oxazepin-2(1H)-yl)prop-2-en-1-one;
1-[(12aR)-9-(2-chloro-6-hydroxyphenyl)-10-fluoro-8-[(pyridin-4-yl)methoxy]-3,4,12,12a-tetrahydro-6H-pyrazino[2,1-c][1,4]benzoxazepin-2(1H)-yl]prop-2-en-1-one;
1-[(12aR)-10-chloro-9-(2-chloro-6-hydroxyphenyl)-8-(2-methoxyethoxy)-3,4,12,12a-tetrahydro-6H-pyrazino[2,1-c][1,4]benzoxazepin-2(1H)-yl]prop-2-en-1-one;
1-[(12aR)-9-(2-Chloro-6-hydroxyphenyl)-10-fluoro-8-[2-(piperidin-1-yl)ethoxy]-3,4,12,12a-tetrahydro-6H-pyrazino[2,1-c][1,4]benzoxazepin-2(1H)-yl]prop-2-en-1-one;
1-[(12aR)-10-Chloro-9-(2-chloro-6-hydroxyphenyl)-8-(prop-1-yn-1-yl)-3,4,12,12a-tetrahydro-6H-pyrazino[2,1-c][1,4]benzoxazepin-2(1H)-yl]prop-2-en-1-one;
1-[(6aR)-4-Chloro-3-(2-chloro-6-hydroxyphenyl)-2-[(²H₃)methyloxy]-6a,7,9,10-tetrahydro-12H-pyrazino[2,1-c]pyrido[2,3-][1,4]oxazepin-8(6H)-yl]prop-2-en-1-one;
1-[(12aR)-10-Chloro-9-(2-chloro-6-hydroxyphenyl)-8-(methoxymethyl)-3,4,12,12a-tetrahydro-6H-pyrazino[2,1-c][1,4]benzoxazepin-2(1H)-yl]prop-2-en-1-one;
1-[(12aR)-9-(2-Chloro-6-hydroxyphenyl)-7-[2-(dimethylamino)ethoxy]-10-fluoro-3,4,12,12a-tetrahydro-6H-pyrazino[2,1-c][1,4]benzoxazepin-2(1H)-yl]prop-2-en-1-one;
1-[(6aR)-4-Chloro-3-(2-chloro-6-hydroxyphenyl)-1-(prop-1-yn-1-yl)-6a,7,9,10-tetrahydro-12H-pyrazino[2,1-c]pyrido[3,4-f][1,4]oxazepin-8(6H)-yl]prop-2-en-1-one; and
1-((6aR)-4-Chloro-3-(2-chloro-6-hydroxyphenyl)-1-ethynyl-6a,7,9,10-tetrahydro-12H-pyrazino[2,1-c]pyrido[3,4-f][1,4]oxazepin-8(6H)-yl)prop-2-en-1-one;
or a pharmaceutically acceptable salt thereof.

29. The method, inhibitor for use or use of claim 7, wherein the small molecule KRAS inhibitor is a compound of Formula (IV):

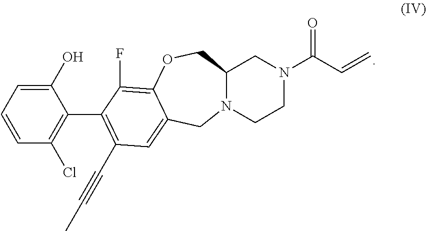 [SMILES: C=CC(=O)N1CCN2Cc3cc(C#CC)c(-c4c(O)cccc4Cl)c(F)c3OC[C@H]2C1]

30. The method, inhibitor for use or use of any one of claims 1 to 29, wherein the ATR inhibitor is an antibody.

31. The method, inhibitor for use or use of any one of claims 1 to 29, wherein the ATR inhibitor is a small molecule ATR inhibitor.

32. The method, inhibitor for use or use of claim 31, wherein the small molecule ATR inhibitor is M6620/VX970 (berzosertib), BAY-1895344 (elimusertib), RP-3500 (camonsertib), ATRN-119, SC0245, ATRN-212, LR-02, M4344 (Gartisertib), M1774, ATG-018, ARTO380, BG-129, JS-123, BKT-300, AZ-20, VE-821, AZD-559 or IMP9064.

33. The method, inhibitor for use or use of claim 31, wherein the small molecule ATR inhibitor is a compound of Formula (V): 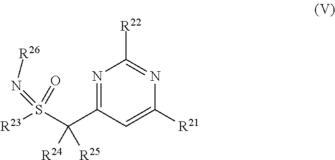
wherein:
R²¹ is selected from morpholin-4-yl and 3-methylmorpholin-4-yl;
R²² is 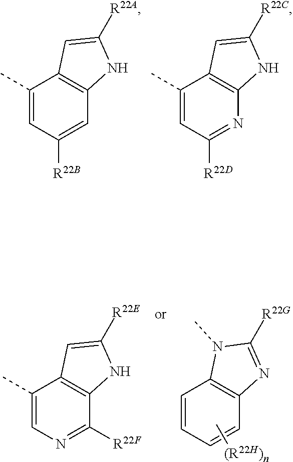
n is 0 or 1;
R^{22A}, R^{22C}, R^{22E} and R^{22F} each independently are hydrogen or methyl;
R^{22B} and R^{22D} each independently are hydrogen or methyl;
R^{22G} is selected from —NHR²⁷ and —NHCOR²⁸;
R^{22H} is fluoro;
R²³ is methyl;
R²⁴ and R²⁵ are each independently hydrogen or methyl, or R²⁴ and R²⁵ together with the atom to which they are attached form Ring A²;
Ring A² is a C₃₋₆ cycloalkyl or a saturated 4-6 membered heterocyclic ring containing one heteroatom selected from O and N;
R²⁶ is hydrogen;
R²⁷ is hydrogen or methyl; and
R²⁸ is methyl,
or a pharmaceutically acceptable salt thereof.

34. The method, inhibitor for use or use of claim 33, wherein R²⁴ and R²⁵ together with the atom to which they are attached form Ring A², and Ring A² is a C₃₋₆ cycloalkyl or a saturated 4-6 heterocyclic ring containing one heteroatom selected from O and N.

35. The method, inhibitor for use or use of claim 33 or 34, wherein Ring A² is a cyclopropyl, tetrahydropyranyl or piperidinyl ring.

36. The method, inhibitor for use or use of any one of claims 33 to 35, wherein R^{22A} is hydrogen; R^{22B} is hydrogen; R^{22C} is hydrogen; R^{22D} is hydrogen; R^{22E} is hydrogen; and R^{22F} is hydrogen.

37. The method, inhibitor for use or use of any one of claims 33 to 36, wherein R²¹ is morpholin-4-yl.

38. The method, inhibitor for use or use of any one of claims 33 to 36, wherein R²¹ is 3-methylmorpholin-4-yl.

39. The method, inhibitor for use or use of claim 33, wherein the compound of Formula (V) is a compound of Formula (Va): 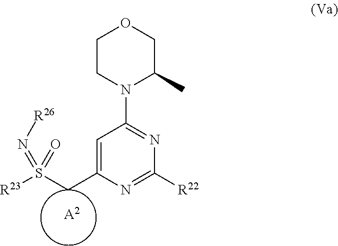
or a pharmaceutically acceptable salt thereof.

40. The method, inhibitor for use or use of claim 39, wherein:
Ring A² is cyclopropyl ring;
R²² is 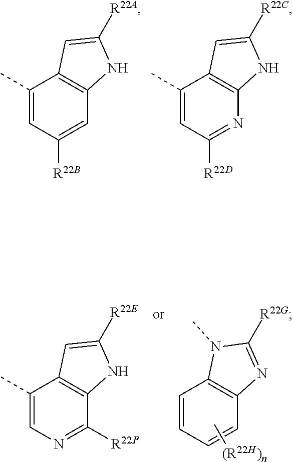
n is 0 or 1;
R^{22A} is hydrogen;
R^{22B} is hydrogen;
R^{22C} is hydrogen;
R^{22D} is hydrogen;
R^{22E} is hydrogen;
R^{22F} is hydrogen;
R^{22G} is —NHR²⁷;
R^{22H} is fluoro;
R²³ is methyl;
R²⁶ is hydrogen; and
R²⁷ is hydrogen or methyl, or a pharmaceutically acceptable salt thereof.

41. The method, inhibitor for use or use of claim 31, wherein the small molecule ATR inhibitor is selected from any one of:
4-{4-[(3R)-3-Methylmorpholin-4-yl]-6-[((R)—S-methylsulfonimidoyl)methyl]pyrimidin-2-yl}-1H-pyrrolo[2,3-b]pyridine;
4-{4-[(3R)-3-Methylmorpholin-4-yl]-6-[1-((S)—S-methylsulfonimidoyl)cyclopropyl]pyrimidin-2-yl}-1H-pyrrolo[2,3-b]pyridine;
4-{4-[(3R)-3-Methylmorpholin-4-yl]-6-[1-((R)—S-methylsulfonimidoyl)cyclopropyl]pyrimidin-2-yl}-1H-pyrrolo[2,3-b]pyridine;
N-Methyl-1-{4-[(3R)-3-methylmorpholin-4-yl]-6-[1-((R)—S-methylsulfonimidoyl)cyclopropyl]pyrimidin-2-yl}-1H-benzimidazol-2-amine;
N-Methyl-1-{4-[(3R)-3-methylmorpholin-4-yl]-6-[1-((S)—S-methylsulfonimidoyl)cyclopropyl]pyrimidin-2-yl}-1H-benzimidazol-2-amine;
4-{4-[(3R)-3-Methylmorpholin-4-yl]-6-[1-((R)—S-methylsulfonimidoyl)cyclopropyl]pyrimidin-2-yl}-1H-indole;
4-{4-[(3R)-3-methylmorpholin-4-yl]-6-[1-((S)—S-methylsulfonimidoyl)cyclopropyl]pyrimidin-2-yl}-1H-indole;
1-{4-[(3R)-3-Methylmorpholin-4-yl]-6-[1-((R)—S-methylsulfonimidoyl)cyclopropyl]pyrimidin-2-yl}-1H-benzimidazol-2-amine;
1-{4-[(3R)-3-methylmorpholin-4-yl]-6-[1-((S)—S-methylsulfonimidoyl)cyclopropyl]pyrimidin-2-yl}-1H-benzimidazol-2-amine;
4-Fluoro-N-methyl-1-{4-[(3R)-3-methylmorpholin-4-yl]-6-[1-((R)—S-methylsulfonimidoyl)cyclopropyl]pyrimidin-2-yl}-1H-benzimidazol-2-amine;
4-fluoro-N-methyl-1-{4-[(3R)-3-methylmorpholin-4-yl]-6-[1-((S)—S-methylsulfonimidoyl)cyclopropyl]pyrimidin-2-yl}-1H-benzimidazol-2-amine;
4-{4-[(3R)-3-Methylmorpholin-4-yl]-6-[1-(S-methylsulfonimidoyl)cyclopropyl]pyrimidin-2-yl}-1H-pyrrolo[2,3-c]pyridine;
N-methyl-1-{4-[1-methyl-1-((S)—S-methylsulfonimidoyl)ethyl]-6-[(3R)-3-methylmorpholin-4-yl]pyrimidin-2-yl}-1H-benzimidazol-2-amine;
N-methyl-1-{4-[i-methyl-1-((R)—S-methylsulfonimidoyl)ethyl]-6-[(3R)-3-methylmorpholin-4-yl]pyrimidin-2-yl}-1H-benzimidazol-2-amine;
N-Methyl-1-{4-[(3R)-3-methylmorpholin-4-yl]-6-[4-((S)—S-methylsulfonimidoyl)tetrahydro-2H-pyran-4-yl]pyrimidin-2-yl}-1H-benzimidazol-2-amine;
N-methyl-1-{4-[(3R)-3-methylmorpholin-4-yl]-6-[4-((R)—S-methylsulfonimidoyl)tetrahydro-2H-pyran-4-yl]pyrimidin-2-yl}-1H-benzimidazol-2-amine;
4-{4-[(3R)-3-Methylmorpholin-4-yl]-6-[4-((S)—S-methylsulfonimidoyl)tetrahydro-2H-pyran-4-yl]pyrimidin-2-yl}-1H-indole;
4-Fluoro-N-methyl-1-{4-[1-methyl-1-((S)—S-methylsulfonimidoyl)ethyl]-6-[(3R)-3-methylmorpholin-4-yl]pyrimidin-2-yl}-1H-benzimidazol-2-amine;
4-fluoro-N-methyl-1-{4-[i-methyl-1-((R)—S-methylsulfonimidoyl)ethyl]-6-[(3R)-3-methylmorpholin-4-yl]pyrimidin-2-yl}-1H-benzimidazol-2-amine;
6-Fluoro-N-methyl-1-{4-[1-methyl-1-((R)—S-methylsulfonimidoyl)ethyl]-6-[(3R)-3-methylmorpholin-4-yl]pyrimidin-2-yl}-1H-benzimidazol-2-amine;
5-Fluoro-N-methyl-1-{4-[1-methyl-1-((R)—S-methylsulfonimidoyl)ethyl]-6-[(3R)-3-methylmorpholin-4-yl]pyrimidin-2-yl}-1H-benzimidazol-2-amine;
5-Fluoro-N-methyl-1-{4-[1-methyl-1-((S)—S-methylsulfonimidoyl)ethyl]-6-[(3R)-3-methylmorpholin-4-yl]pyrimidin-2-yl}-1H-benzimidazol-2-amine;
6-fluoro-N-methyl-1-{4-[i-methyl-1-((S)—S-methylsulfonimidoyl)ethyl]-6-[(3R)-3-methylmorpholin-4-yl]pyrimidin-2-yl}-1H-benzimidazol-2-amine;
6-Fluoro-N-methyl-1-{4-[(3R)-3-methylmorpholin-4-yl]-6-[1-((R)—S-methylsulfonimidoyl)cyclopropyl]pyrimidin-2-yl}-1H-benzimidazol-2-amine;
5-fluoro-N-methyl-1-{4-[(3R)-3-methylmorpholin-4-yl]-6-[1-((R)—S-methylsulfonimidoyl)cyclopropyl]pyrimidin-2-yl}-1H-benzimidazol-2-amine;
5-fluoro-N-methyl-1-{4-[(3R)-3-methylmorpholin-4-yl]-6-[1-((S)—S-methylsulfonimidoyl)cyclopropyl]pyrimidin-2-yl}-1H-benzimidazol-2-amine; and
6-fluoro-N-methyl-1-{4-[(3R)-3-methylmorpholin-4-yl]-6-[1-((S)—S-methylsulfonimidoyl)cyclopropyl]pyrimidin-2-yl}-1H-benzimidazol-2-amine, or a pharmaceutically acceptable salt thereof.

42. The method, inhibitor for use or use of claim 31, wherein the small molecule ATR inhibitor is a compound of Formula (VI) (ceralasertib): 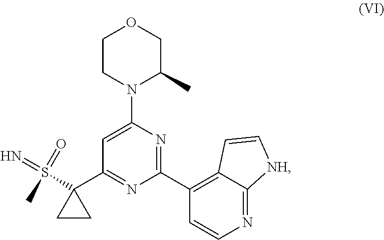 [SMILES: C[C@@H]1COCCN1c1cc(C2([S@](C)(=N)=O)CC2)nc(-c2ccnc3[nH]ccc23)n1]
or a pharmaceutically acceptable salt thereof.

43. The method of claim 1, inhibitor for use of claim 2 or claim 3 or use of claim 4, wherein the (i) KRAS inhibitor, and (ii) ATR inhibitor is one of the following combinations:

| Combo | KRAS | ATR |
|---|---|---|
| # | inhibitor | inhibitor |
| 1 | sotorasib | berzosertib |
| 2 | sotorasib | elimusertib |
| 3 | sotorasib | camonsertib |
| 4 | sotorasib | gartisertib |
| 5 | sotorasib | M1774 |
| 6 | sotorasib | compound of |
| | | Formula (V) |
| 7 | sotorasib | ceralasertib |
| 8 | sotorasib | ATG-018 |
| 9 | sotorasib | ART0380 |
| 10 | sotorasib | IMP9064 |
| 11 | adagrasib | berzosertib |
| 12 | adagrasib | elimusertib |
| 13 | adagrasib | camonsertib |
| 14 | adagrasib | gartisertib |
| 15 | adagrasib | M1774 |
| 16 | adagrasib | compound of |
| | | Formula (V) |
| 17 | adagrasib | ceralasertib |
| 18 | adagrasib | ATG-018 |
| 19 | adagrasib | ART0380 |
| 20 | adagrasib | IMP9064 |
| 21 | compound of | berzosertib |
| | Formula (I) | |
| 22 | compound of | elimusertib |
| | Formula (I) | |
| 23 | compound of | camonsertib |
| | Formula (I) | |
| 24 | compound of | gartisertib |
| | Formula (I) | |
| 25 | compound of | M1774 |
| | Formula (I) | |
| 26 | compound of | compound of |
| | Formula (I) | Formula (V) |
| 27 | compound of | ceralasertib |
| | Formula (I) | |
| 28 | compound of | ATG-018 |
| | Formula (I) | |
| 29 | compound of | ART0380 |
| | Formula (I) | |
| 30 | compound of | IMP9064 |
| | Formula (I) | |
| 31 | compound of | berzosertib |
| | Formula (II) | |
| 32 | compound of | elimusertib |
| | Formula (II) | |
| 33 | compound of | camonsertib |
| | Formula (II) | |
| 34 | compound of | gartisertib |
| | Formula (II) | |
| 35 | compound of | M1774 |
| | Formula (II) | |
| 36 | compound of | compound of |
| | Formula (II) | Formula (V) |
| 37 | compound of | ceralasertib |
| | Formula (II) | |
| 38 | compound of | ATG-018 |
| | Formula (II) | |
| 39 | compound of | ART0380 |
| | Formula (II) | |
| 40 | compound of | IMP9064 |
| | Formula (II) | |
| 41 | compound of | berzosertib |
| | Formula (III) | |
| 42 | compound of | elimusertib |
| | Formula (III) | |
| 43 | compound of | camonsertib |
| | Formula (III) | |
| 44 | compound of | gartisertib |
| | Formula (III) | |
| 45 | compound of | M1774 |
| | Formula (III) | |
| 45 | compound of | compound of |
| | Formula (III) | Formula (V) |
| 46 | compound of | ceralasertib |
| | Formula (III) | |
| 47 | compound of | ATG-018 |
| | Formula (III) | |
| 49 | compound of | ART0380 |
| | Formula (III) | |
| 50 | compound of | IMP9064 |
| | Formula (III) | |
| 51 | compound of | berzosertib |
| | Formula (IV) | |
| 52 | compound of | elimusertib |
| | Formula (IV) | |
| 53 | compound of | camonsertib |
| | Formula (IV) | |
| 54 | compound of | gartisertib |
| | Formula (IV) | |
| 55 | compound of | M1774 |
| | Formula (IV) | |
| 56 | compound of | compound of |
| | Formula (IV) | Formula (V) |
| 57 | compound of | ceralasertib |
| | Formula (IV) | |
| 58 | compound of | ATG-018 |
| | Formula (IV) | |
| 59 | compound of | ART0380 |
| | Formula (IV) | |
| 60 | compound of | IMP9064 |
| | Formula (IV) | |
| 61 | D35-001 | berzosertib |
| 62 | D35-001 | elimusertib |
| 63 | D35-001 | camonsertib |
| 64 | D35-001 | gartisertib |
| 65 | D35-001 | M1774 |
| 66 | D35-001 | compound of |
| | | Formula (V) |
| 67 | D35-001 | ceralasertib |
| 68 | D35-001 | ATG-018 |
| 68 | D35-001 | ART0380 |
| 70 | D35-001 | IMP9064 |
| 71 | JAB-21822 | berzosertib |
| 72 | JAB-21822 | elimusertib |
| 73 | JAB-21822 | camonsertib |
| 74 | JAB-21822 | gartisertib |
| 75 | JAB-21822 | M1774 |
| 76 | JAB-21822 | compound of |
| | | Formula (V) |
| 77 | JAB-21822 | ceralasertib |
| 78 | JAB-21822 | ATG-018 |
| 79 | JAB-21822 | ART0380 |
| 80 | JAB-21822 | IMP9064 |
| 81 | HBI-2438 | berzosertib |
| 82 | HBI-2438 | elimusertib |
| 83 | HBI-2438 | camonsertib |
| 84 | HBI-2438 | gartisertib |
| 85 | HBI-2438 | M1774 |
| 86 | HBI-2438 | compound of |
| | | Formula (V) |
| 87 | HBI-2438 | ceralasertib |
| 88 | HBI-2438 | ATG-018 |
| 89 | HBI-2438 | ART0380 |
| 90 | HBI-2438 | IMP9064 |
| 91 | YL-15293 | berzosertib |
| 92 | YL-15293 | elimusertib |
| 93 | YL-15293 | camonsertib |
| 94 | YL-15293 | gartisertib |
| 95 | YL-15293 | M1774 |
| 96 | YL-15293 | compound of |
| | | Formula (V) |
| 97 | YL-15293 | ceralasertib |
| 98 | YL-15293 | ATG-018 |
| 99 | YL-15293 | ART0380 |
| 100 | YL-15293 | IMP9064 |
| 101 | GEC255 | berzosertib |
| 102 | GEC255 | elimusertib |
| 103 | GEC255 | camonsertib |
| 104 | GEC255 | gartisertib |
| 105 | GEC255 | M1774 |
| 106 | GEC255 | compound of |
| | | Formula (V) |
| 107 | GEC255 | ceralasertib |
| 108 | GEC255 | ATG-018 |
| 109 | GEC255 | ART0380 |
| 110 | GEC255 | IMP9064 |
| 111 | IBI351 | berzosertib |
| 112 | IBI351 | elimusertib |
| 113 | IBI351 | camonsertib |
| 114 | IBI351 | gartisertib |
| 115 | IBI351 | M1774 |
| 116 | IBI351 | compound of |
| | | Formula (V) |
| 117 | IBI351 | ceralasertib |
| 118 | IBI351 | ATG-018 |
| 119 | IBI351 | ART0380 |
| 120 | IBI351 | IMP9064 |
| 121 | RMC-6291 | berzosertib |
| 122 | RMC-6291 | elimusertib |
| 123 | RMC-6291 | camonsertib |
| 124 | RMC-6291 | gartisertib |
| 125 | RMC-6291 | M1774 |
| 126 | RMC-6291 | compound of |
| | | Formula (V) |
| 127 | RMC-6291 | ceralasertib |
| 128 | RMC-6291 | ATG-018 |
| 129 | RMC-6291 | ART0380 |
| 130 | RMC-6291 | IMP9064 |
| 131 | MK-1084 | berzosertib |
| 132 | MK-1084 | elimusertib |
| 133 | MK-1084 | camonsertib |
| 134 | MK-1084 | gartisertib |
| 135 | MK-1084 | M1774 |
| 136 | MK-1084 | compound of |
| | | Formula (V) |
| 137 | MK-1084 | ceralasertib |
| 138 | MK-1084 | ATG-018 |
| 139 | MK-1084 | ART0380 |
| 140 | MK-1084 | IMP9064 |
| 141 | MRTX1133 | berzosertib |
| 142 | MRTX1133 | elimusertib |
| 143 | MRTX1133 | camonsertib |
| 144 | MRTX1133 | gartisertib |
| 145 | MRTX1133 | M1774 |
| 146 | MRTX1133 | compound of |
| | | Formula (V) |
| 147 | MRTX1133 | ceralasertib |
| 148 | MRTX1133 | ATG-018 |
| 149 | MRTX1133 | ART0380 |
| 150 | MRTX1133 | IMP9064 |
| 151 | HRS-4642 | berzosertib |
| 152 | HRS-4642 | elimusertib |
| 153 | HRS-4642 | camonsertib |
| 154 | HRS-4642 | gartisertib |
| 155 | HRS-4642 | M1774 |
| 156 | HRS-4642 | compound of |
| | | Formula (V) |
| 157 | HRS-4642 | ceralasertib |
| 158 | HRS-4642 | ATG-018 |
| 159 | HRS-4642 | ART0380 |
| 160 | HRS-4642 | IMP9064 |
| 161 | RMC-9805 | berzosertib |
| 163 | RMC-9805 | elimusertib |
| 163 | RMC-9805 | camonsertib |
| 164 | RMC-9805 | gartisertib |
| 165 | RMC-9805 | M1774 |
| 166 | RMC-9805 | compound of |
| | | Formula (V) |
| 167 | RMC-9805 | ceralasertib |
| 168 | RMC-9805 | ATG-018 |
| 169 | RMC-9805 | ART0380 |
| 170 | RMC-9805 | IMP9064 |
| 171 | Ly3537982 | berzosertib |
| 172 | Ly3537982 | elimusertib |
| 173 | Ly3537982 | camonsertib |
| 174 | Ly3537982 | gartisertib |
| 175 | Ly3537982 | M1774 |
| 176 | Ly3537982 | compound of |
| | | Formula (V) |
| 177 | Ly3537982 | ceralasertib |
| 178 | Ly3537982 | ATG-018 |
| 179 | Ly3537982 | ART0380 |
| 180 | Ly3537982 | IMP9064 |
| 181 | GDC-6036 | berzosertib |
| 182 | GDC-6036 | elimusertib |
| 183 | GDC-6036 | camonsertib |
| 184 | GDC-6036 | gartisertib |
| 185 | GDC-6036 | M1774 |
| 186 | GDC-6036 | compound of |
| | | Formula (V) |
| 187 | GDC-6036 | ceralasertib |
| 188 | GDC-6036 | ATG-018 |
| 189 | GDC-6036 | ART0380 |
| 190 | GDC-6036 | IMP9064 |
| 191 | D-1553 | berzosertib |
| 192 | D-1553 | elimusertib |
| 193 | D-1553 | camonsertib |
| 194 | D-1553 | gartisertib |
| 195 | D-1553 | M1774 |
| 196 | D-1553 | compound of |
| | | Formula (V) |
| 197 | D-1553 | ceralasertib |
| 198 | D-1553 | ATG-018 |
| 199 | D-1553 | ART0380 |
| 200 | D-1553 | IMP9064 |
| 201 | JDQ443 | berzosertib |
| 202 | JDQ443 | elimusertib |
| 203 | JDQ443 | camonsertib |
| 204 | JDQ443 | gartisertib |
| 205 | JDQ443 | M1774 |
| 206 | JDQ443 | compound of |
| | | Formula (V) |
| 207 | JDQ443 | ceralasertib |
| 208 | JDQ443 | ATG-018 |
| 209 | JDQ443 | ART0380 |
| 210 | JDQ443 | IMP9064 |
| 211 | BI1823911 | berzosertib |
| 212 | BI1823911 | elimusertib |
| 213 | BI1823911 | camonsertib |
| 214 | BI1823911 | gartisertib |
| 215 | BI1823911 | M1774 |
| 216 | BI1823911 | compound of |
| | | Formula (V) |
| 217 | BI1823911 | ceralasertib |
| 218 | BI1823911 | ATG-018 |
| 219 | BI1823911 | ART0380 |
| 220 | BI1823911 | IMP9064 |
| 221 | GFH925 | berzosertib |
| 222 | GFH925 | elimusertib |
| 223 | GFH925 | camonsertib |
| 224 | GFH925 | gartisertib |
| 225 | GFH925 | M1774 |
| 226 | GFH925 | compound of |
| | | Formula (V) |
| 227 | GFH925 | ceralasertib |
| 228 | GFH925 | ATG-018 |
| 229 | GFH925 | ART0380 |
| 230 | GFH925 | IMP9064 |
| 231 | JAB-2200 | berzosertib |
| 232 | JAB-2200 | elimusertib |
| 233 | JAB-2200 | camonsertib |
| 234 | JAB-2200 | gartisertib |
| 235 | JAB-2200 | M1774 |
| 236 | JAB-2200 | compound of |
| | | Formula (V) |
| 237 | JAB-2200 | ceralasertib |
| 238 | JAB-2200 | ATG-018 |
| 239 | JAB-2200 | ART0380 |
| 240 | JAB-2200 | IMP9064 |
| 241 | Vrtx153 | berzosertib |
| 242 | Vrtx153 | elimusertib |
| 243 | Vrtx153 | camonsertib |
| 244 | Vrtx153 | gartisertib |
| 245 | Vrtx153 | M1774 |
| 246 | Vrtx153 | compound of |
| | | Formula (V) |
| 247 | Vrtx153 | ceralasertib |
| 248 | Vrtx153 | ATG-018 |
| 249 | Vrtx153 | ART0380 |
| 250 | Vrtx153 | IMP9064 |
| 252 | ERAS-4 | berzosertib |
| 252 | ERAS-4 | elimusertib |
| 253 | ERAS-4 | camonsertib |
| 254 | ERAS-4 | gartisertib |
| 255 | ERAS-4 | M1774 |
| 256 | ERAS-4 | compound of |
| | | Formula (V) |
| 257 | ERAS-4 | ceralasertib |
| 258 | ERAS-4 | ATG-018 |
| 259 | ERAS-4 | ART0380 |
| 260 | ERAS-4 | IMP9064 |
| 261 | ASP3082 | berzosertib |
| 262 | ASP3082 | elimusertib |
| 263 | ASP3082 | camonsertib |
| 264 | ASP3082 | gartisertib |
| 265 | ASP3082 | M1774 |
| 266 | ASP3082 | compound of |
| | | Formula (V) |
| 267 | ASP3082 | ceralasertib |
| 268 | ASP3082 | ATG-018 |
| 269 | ASP3082 | ART0380 |
| 270 | ASP3082 | IMP9064 |
| 271 | RMC-0708 | berzosertib |
| 272 | RMC-0708 | elimusertib |
| 273 | RMC-0708 | camonsertib |
| 274 | RMC-0708 | gartisertib |
| 275 | RMC-0708 | M1774 |
| 276 | RMC-0708 | compound of |
| | | Formula (V) |
| 277 | RMC-0708 | ceralasertib |
| 278 | RMC-0708 | ATG-018 |
| 279 | RMC-0708 | ART0380 |
| 280 | RMC-0708 | IMP9064 |
| 281 | RMC-8839 | berzosertib |
| 282 | RMC-8839 | elimusertib |
| 283 | RMC-8839 | camonsertib |
| 284 | RMC-8839 | gartisertib |
| 285 | RMC-8839 | M1774 |
| 286 | RMC-8839 | compound of |
| | | Formula (V) |
| 287 | RMC-8839 | ceralasertib |
| 288 | RMC-8839 | ATG-018 |
| 289 | RMC-8839 | ART0380 |
| 290 | RMC-8839 | IMP9064 |
| 291 | RMC-6291 | berzosertib |
| 292 | RMC-6291 | elimusertib |
| 293 | RMC-6291 | camonsertib |
| 294 | RMC-6291 | gartisertib |
| 295 | RMC-6291 | M1774 |
| 296 | RMC-6291 | compound of |
| | | Formula (V) |
| 297 | RMC-6291 | ceralasertib |
| 298 | RMC-6291 | ATG-018 |
| 299 | RMC-6291 | ART0380 |
| 300 | RMC-6291 | IMP9064 |
| 301 | JAB-23400 | berzosertib |
| 302 | JAB-23400 | elimusertib |
| 303 | JAB-23400 | camonsertib |
| 304 | JAB-23400 | gartisertib |
| 305 | JAB-23400 | M1774 |
| 306 | JAB-23400 | compound of |
| | | Formula (V) |
| 307 | JAB-23400 | ceralasertib |
| 308 | JAB-23400 | ATG-018 |
| 309 | JAB-23400 | ART0380 |
| 310 | JAB-23400 | IMP9064 |
| 311 | TEB-17231 | berzosertib |
| 312 | TEB-17231 | elimusertib |
| 313 | TEB-17231 | camonsertib |
| 314 | TEB-17231 | gartisertib |
| 315 | TEB-17231 | M1774 |
| 316 | TEB-17231 | compound of |
| | | Formula (V) |
| 317 | TEB-17231 | ceralasertib |
| 318 | TEB-17231 | ATG-018 |
| 319 | TEB-17231 | ART0380 |
| 320 | TEB-17231 | IMP9064 |
| 321 | QTX3046 | berzosertib |
| 322 | QTX3046 | elimusertib |
| 323 | QTX3046 | camonsertib |
| 324 | QTX3046 | gartisertib |
| 325 | QTX3046 | M1774 |
| 326 | QTX3046 | compound of |
| | | Formula (V) |
| 327 | QTX3046 | ceralasertib |
| 328 | QTX3046 | ATG-018 |
| 329 | QTX3046 | ART0380 |
| 330 | QTX3046 | IMP9064 |

44. The method of claim 1, inhibitor for use of claim 2 or claim 3 or use of claim 4, wherein the (i) KRAS inhibitor is a compound of Formula (II), and (ii) ATR inhibitor is ceralasertib.

45. The method of claim 1, inhibitor for use of claim 2 or claim 3 or use of claim 4, wherein the (i) KRAS inhibitor is a compound of Formula (IV), and (ii) ATR inhibitor is ceralasertib.

46. The method, inhibitor for use or use of any one of claims 1 to 45, wherein the subject has a KRAS, NRAS or HRAS mutation.

47. The method, inhibitor for use or use of any one of claims 1 to 45, wherein the subject has a KRAS mutation.

48. The method, inhibitor for use or use of claim 47, wherein the KRAS mutation is a mutation at codon 12, codon 13 and/or codon 61.

49. The method, inhibitor for use or use of claim 47, wherein the KRAS mutation is a mutation at codon 12.

50. The method, inhibitor for use or use of claim 47, wherein the KRAS mutation is a G12C mutation.

51. The method, inhibitor for use or use of any one of claims 1 to 50, wherein the subject has lung cancer, colorectal cancer, pancreatic cancer, esophagogastric cancer, endometrial cancer, cholangiocarcinoma, or combination thereof.

52. The method, inhibitor for use or use of any one of claims 1 to 51, wherein the subject is a human subject.

53. The method, inhibitor for use or use of any one of claims 1 to 52, wherein the subject has lung cancer, colorectal cancer, pancreatic cancer, or combination thereof.

54. A method of modulating the adaptive immune response in a subject, the method comprising administering a composition comprising:
a. a KRAS inhibitor, and
b. an ATR inhibitor.

55. A method of reducing the volume of a cancerous tumor in a subject, the method comprising administering a composition comprising:
a. a KRAS inhibitor, and
b. an ATR inhibitor.

56. A method of treating a subject in need thereof, the method comprising administering a composition comprising:
a. a KRAS inhibitor, and
b. an ATR inhibitor.
wherein the subject has a disorder mediated by a KRAS, NRAS or HRAS G12C mutation.

57. A composition comprising:
a. a KRAS inhibitor, and
b. an ATR inhibitor.

58. The composition of claim 57, wherein the KRAS inhibitor is sotorasib, adagrasib, Ly3537982, GDC-6036, D-1553, JDQ443, BI1823911, GFH925, D35-001, JAB-21822, HBI-2438, YL-15293, GEC255, IBI351, RMC-6291, MK-1084, MRTX1133, HRS-4642, RMC-9805, JAB-2200, Vrtx153, ERAS-4, ASP3082, RMC-0708, RMC-8839, RMC-6291, JAB-23400, TEB-17231, QTX3046, a compound of Formula (I), a compound of Formula (II), a compound of Formula (III), or a compound of Formula (IV).

59. The composition of claim 57 or 58, wherein the ATR inhibitor is M6620/VX970 (berzosertib), BAY-1895344 (elimusertib), RP-3500 (camonsertib), ATRN-119, SC0245, ATRN-212, LR-02, M4344 (Gartisertib), M1774, ATG-018, ART0380, BG-129, JS-123, BKT-300, AZ-20, VE-821, AZD-5597, or IMP9064, a compound of Formula (V), or a compound of Formula (VI).

60. The composition of any one of claims 57 to 59, wherein the composition is a liquid.

61. The composition of any one of claims 57 to 59, wherein the composition is a solid.

62. The composition of any one of claims 57 to 59, wherein the composition is a solid oral dosage form.

63. The composition of any one of claims 57 to 62, wherein the composition further comprises a pharmaceutically acceptable excipient, diluent or carrier.

64. Use of the composition of any one of claims 57 to 63, or a pharmaceutical acceptable salt thereof, for the manufacture of a medicament, optionally a medicament for the treatment of cancer.

65. A kit comprising:
a. a first container comprising a first composition comprising a KRAS inhibitor, and
b. a second container comprising a second composition comprising an ATR inhibitor.

66. The kit of claim 65, wherein the KRAS inhibitor is sotorasib, adagrasib, Ly3537982, GDC-6036, D-1553, JDQ443, BI1823911, GFH925, D35-001, JAB-21822, HBI-2438, YL-15293, GEC255, IBI351, RMC-6291, MK-1084, MRTX1133, HRS-4642, RMC-9805, JAB-2200, Vrtx153, ERAS-4, ASP3082, RMC-0708, RMC-8839, RMC-6291, JAB-23400, TEB-17231, QTX3046, a compound of Formula (I), a compound of Formula (II), a compound of Formula (III), or a compound of Formula (IV).

67. The kit of claim 65 or 66, wherein the ATR inhibitor is M6620/VX970 (berzosertib), BAY-1895344 (elimusertib), RP-3500 (camonsertib), ATRN-119, SC0245, ATRN-212, LR-02, M4344 (Gartisertib), M1774, ATG-018, ARTO380, BG-129, JS-123, BKT-300, AZ-20, VE-821, AZD-5597, or IMP9064, a compound of Formula (V), or a compound of Formula (VI).

68. The kit of any one of claims 65 to 67, wherein the first composition, the second composition, or both the first composition and the second composition is/are a liquid.

69. The kit of any one of claims 65 to 67, wherein the first composition, the second composition, or both the first composition and the second composition is/are a solid.

70. The kit of any one of claims 65 to 67, wherein the first composition, the second composition, or both the first composition and the second composition is/are a solid oral dosage form.

71. The kit of any one of claims 65 to 70, wherein the first composition, the second composition, or both the first composition and the second composition further comprise a pharmaceutically acceptable excipient, diluent or carrier.


SEQUENCE LISTING (3 sequences)

SEQ ID NO: 1 - protein, 189 aa, synthetic construct [sequence omitted]

SEQ ID NO: 2 - protein, 189 aa, synthetic construct
MTEYKLVVVGAGGVGKSALTIQLIQNHFVDEYDPTIEDSYRKQVVIDGETCLLDILDTAGQEEYSAMRDQYMRTGEGFLCVFAINNSKSFADINLYREQIKRVKDSDDVPMVLVGNKCDLPTRTVDTKQAHELAKSYGIPFIETSAKTRQGVEDAFYTLVREIRQYRMKKLNSSDDGTQGCMGLPCVVM

SEQ ID NO: 3 - protein, 189 aa, synthetic construct
MTEYKLVVVGAGGVGKSALTIQLIQNHFVDEYDPTIEDSYRKQVVIDGETCLLDILDTAGQEEYSAMRDQYMRTGEGFLCVFAINNTKSFEDIHQYREQIKRVKDSDDVPMVLVGNKCDLAARTVESRQAQDLARSYGIPYIETSAKTRQGVEDAFYTLVREIRQHKLRKLNPPDESGPGCMSCKCVLS